SEQUENCE LISTING (116 sequences)

SEQ ID NO: 1 - protein, 107 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 107 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 107 aa, Homo sapiens
GQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 4 - protein, 107 aa, Homo sapiens
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 5 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 103 aa, Homo sapiens
PEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTID

SEQ ID NO: 7 - protein, 103 aa, Homo sapiens
PEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTID

SEQ ID NO: 8 - protein, 107 aa, Homo sapiens
PAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLE

SEQ ID NO: 9 - protein, 105 aa, Homo sapiens
PRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTS

SEQ ID NO: 10 - protein, 106 aa, Homo sapiens
QDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKG

SEQ ID NO: 11 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 12 - protein, 109 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 13 - protein, 110 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 14 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 15 - protein, 96 aa, Homo sapiens
PRLSLHRPALEDLLLGSEANLTCTLTGLRDASGVTFTWTPSSGKSAVQGPPERDLCGCYSVSSVLPGCAEPWNHGKTFTCTAAYPESKTPLTATLS

SEQ ID NO: 16 - protein, 96 aa, Homo sapiens
PRLSLHRPALEDLLLGSEANLTCTLTGLRDASGATFTWTPSSGKSAVQGPPERDLCGCYSVSSVLPGCAQPWNHGETFTCTAAHPELKTPLTANIT

SEQ ID NO: 17 - protein, 89 aa, Homo sapiens
PAVQDLWLRDKATFTCFVVGSDLKDAHLTWEVAGKVPTGGVEEGLLERHSNGSQSQHSRLTLPRSLWNAGTSVTCTLNHPSLPPQRLMA

SEQ ID NO: 18 - protein, 99 aa, Homo sapiens
PTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCA

SEQ ID NO: 19 - protein, 112 aa, Homo sapiens
IAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPD

SEQ ID NO: 20 - protein, 15 aa, Homo sapiens
EPKSCDKTHTCPPCP

SEQ ID NO: 21 - protein, 12 aa, Homo sapiens
ERKCCVECPPCP

SEQ ID NO: 22 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 23 - protein, 12 aa, Homo sapiens
ESKYGPPCPSCP

SEQ ID NO: 24 - protein, 107 aa, synthetic construct
  VARIANT 15: note = X can be R or Q
  VARIANT 16: note = X can be E or D
  VARIANT 18: note = X can be M or L
  VARIANT 38: note = X can be A or S
  VARIANT 44: note = X can be N or S
  VARIANT 52: note = X can be K or N
  VARIANT 57: note = X can be V or M
  VARIANT 69: note = X can be K or R
  VARIANT 79: note = X can be Q or E
  VARIANT 82: note = X can be V or I
  VARIANT 95: note = X can be H or R
  VARIANT 96: note = X can be Y or F
  VARIANT 105: note = X can be P or L
GQPREPQVYTLPPSXXEXTKNQVSLWCLVEGFYPSDIXVEWESXGQPENNYXTTPPXLDSDGSFFLYSXLTVDKSRWQXGNXFSCSVMHEALHNXXTQKSLSLSXGK

SEQ ID NO: 25 - protein, 107 aa, synthetic construct
  VARIANT 15: note = X can be R or Q
  VARIANT 16: note = X can be E or D
  VARIANT 18: note = X can be M or L
  VARIANT 38: note = X can be A or S
  VARIANT 44: note = X can be N or S
  VARIANT 52: note = X can be K or N
  VARIANT 57: note = X can be V or M
  VARIANT 69: note = X can be K or R
  VARIANT 79: note = X can be Q or E
  VARIANT 82: note = X can be V or I
  VARIANT 95: note = X can be H or R
  VARIANT 96: note = X can be Y or F
  VARIANT 105: note = X can be P or L
GQPREPQVYTLPPSXXKXTKNQVSLSCAVKGFYPSDIXVEWESXGQPENNYXTTPPXLDSDGSFFLVSXLTVDKSRWQXGNXFSCSVMHEALHNXXTQKSLSLSXGK

SEQ ID NO: 26 - protein, 107 aa, synthetic construct
  VARIANT 15: note = X can be R or Q
  VARIANT 16: note = X can be E or D
  VARIANT 18: note = X can be M or L
  VARIANT 38: note = X can be A or S
  VARIANT 44: note = X can be N or S
  VARIANT 52: note = X can be K or N
  VARIANT 57: note = X can be V or M
  VARIANT 69: note = X can be K or R
  VARIANT 79: note = X can be Q or E
  VARIANT 82: note = X can be V or I
  VARIANT 95: note = X can be H or R
  VARIANT 96: note = X can be Y or F
  VARIANT 105: note = X can be P or L
GQPREPQVYTLPPCXXEXTKNQVSLWCLVEGFYPSDIXVEWESXGQPENNYXTTPPXLDSDGSFFLYSXLTVDKSRWQXGNXFSCSVMHEALHNXXTQKSLSLSXGK

SEQ ID NO: 27 - protein, 107 aa, synthetic construct
  VARIANT 15: note = X can be R or Q
  VARIANT 16: note = X can be E or D
  VARIANT 18: note = X can be M or L
  VARIANT 38: note = X can be A or S
  VARIANT 44: note = X can be N or S
  VARIANT 52: note = X can be K or N
  VARIANT 57: note = X can be V or M
  VARIANT 69: note = X can be K or R
  VARIANT 79: note = X can be Q or E
  VARIANT 82: note = X can be V or I
  VARIANT 95: note = X can be H or R
  VARIANT 96: note = X can be Y or F
  VARIANT 105: note = X can be P or L
GQPREPQVCTLPPSXXKXTKNQVSLSCAVKGFYPSDIXVEWESXGQPENNYXTTPPXLDSDGSFFLVSXLTVDKSRWQXGNXFSCSVMHEALHNXXTQKSLSLSXGK

SEQ ID NO: 28 - protein, 107 aa, synthetic construct
GQPREPQVYTLPPSRDELTKNQVSLWCLVEGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 107 aa, synthetic construct
GQPREPQVYTLPPSRDKLTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 30 - protein, 107 aa, synthetic construct
GQPREPQVYTLPPCRDELTKNQVSLWCLVEGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 107 aa, synthetic construct
GQPREPQVCTLPPSRDKLTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 32 - protein, 107 aa, synthetic construct
GQPREPQVYTLPPSREEMTKNQVSLWCLVEGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 107 aa, synthetic construct
GQPREPQVYTLPPSREKMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 34 - protein, 107 aa, synthetic construct
GQPREPQVYTLPPCREEMTKNQVSLWCLVEGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 107 aa, synthetic construct
GQPREPQVCTLPPSREKMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 107 aa, synthetic construct
GQPREPQVYTLPPSREEMTKNQVSLWCLVEGFYPSDISVEWESNGQPENNYKTTPPMLDSDGSFFLYSKLTVDKSRWQQGNVFSCSVMHEALHNHYTQKSLSLSPGK

SEQ ID NO: 37 - protein, 107 aa, synthetic construct
GQPREPQVYTLPPSREKMTKNQVSLSCAVKGFYPSDISVEWESNGQPENNYKTTPPMLDSDGSFFLVSKLTVDKSRWQQGNVFSCSVMHEALHNHYTQKSLSLSPGK

SEQ ID NO: 38 - protein, 107 aa, synthetic construct
GQPREPQVYTLPPCREEMTKNQVSLWCLVEGFYPSDISVEWESNGQPENNYKTTPPMLDSDGSFFLYSKLTVDKSRWQQGNVFSCSVMHEALHNHYTQKSLSLSPGK

SEQ ID NO: 39 - protein, 107 aa, synthetic construct
GQPREPQVCTLPPSREKMTKNQVSLSCAVKGFYPSDISVEWESNGQPENNYKTTPPMLDSDGSFFLVSKLTVDKSRWQQGNVFSCSVMHEALHNHYTQKSLSLSPGK

SEQ ID NO: 40 - protein, 107 aa, synthetic construct
GQPREPQVYTLPPSREEMTKNQVSLWCLVEGFYPSDIAVEWESSGQPENNYNTTPPMLDSDGSFFLYSKLTVDKSRWQQGNIFSCSVMHEALHNRFTQKSLSLSPGK

SEQ ID NO: 41 - protein, 107 aa, synthetic construct
GQPREPQVYTLPPSREKMTKNQVSLSCAVKGFYPSDIAVEWESSGQPENNYNTTPPMLDSDGSFFLVSKLTVDKSRWQQGNIFSCSVMHEALHNRFTQKSLSLSPGK

SEQ ID NO: 42 - protein, 107 aa, synthetic construct
GQPREPQVYTLPPCREEMTKNQVSLWCLVEGFYPSDIAVEWESSGQPENNYNTTPPMLDSDGSFFLYSKLTVDKSRWQQGNIFSCSVMHEALHNRFTQKSLSLSPGK

SEQ ID NO: 43 - protein, 107 aa, synthetic construct
GQPREPQVCTLPPSREKMTKNQVSLSCAVKGFYPSDIAVEWESSGQPENNYNTTPPMLDSDGSFFLVSKLTVDKSRWQQGNIFSCSVMHEALHNRFTQKSLSLSPGK

SEQ ID NO: 44 - protein, 107 aa, synthetic construct
GQPREPQVYTLPPSQEEMTKNQVSLWCLVEGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 107 aa, synthetic construct
GQPREPQVYTLPPSQEKMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 46 - protein, 107 aa, synthetic construct
GQPREPQVYTLPPCQEEMTKNQVSLWCLVEGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 107 aa, synthetic construct
GQPREPQVCTLPPSQEKMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 48 - protein, 107 aa, synthetic construct
  VARIANT 15: note = X can be R or Q
  VARIANT 16: note = X can be E or D
  VARIANT 18: note = X can be M or L
  VARIANT 38: note = X can be A or S
  VARIANT 44: note = X can be N or S
  VARIANT 52: note = X can be K or N
  VARIANT 57: note = X can be V or M
  VARIANT 69: note = X can be K or R
  VARIANT 79: note = X can be Q or E
  VARIANT 82: note = X can be V or I
  VARIANT 95: note = X can be H or R
  VARIANT 96: note = X can be Y or F
  VARIANT 105: note = X can be P or L
GQPREPQVYTLPPSXXEXTKNQVSLWCLVKGFYPSDIXVEWESXGQPENNYXTTPPXLDSDGSFFLYSXLTVDKSRWQXGNXFSCSVMHEALHNXXTQKSLSLSXGK

SEQ ID NO: 49 - protein, 107 aa, synthetic construct
  VARIANT 15: note = X can be R or Q
  VARIANT 16: note = X can be E or D
  VARIANT 18: note = X can be M or L
  VARIANT 38: note = X can be A or S
  VARIANT 44: note = X can be N or S
  VARIANT 52: note = X can be K or N
  VARIANT 57: note = X can be V or M
  VARIANT 69: note = X can be K or R
  VARIANT 79: note = X can be Q or E
  VARIANT 82: note = X can be V or I
  VARIANT 95: note = X can be H or R
  VARIANT 96: note = X can be Y or F
  VARIANT 105: note = X can be P or L
GQPREPQVYTLPPSXXEXTKNQVSLSCAVKGFYPSDIXVEWESXGQPENNYXTTPPXLDSDGSFFLVSXLTVDKSRWQXGNXFSCSVMHEALHNXXTQKSLSLSXGK

SEQ ID NO: 50 - protein, 107 aa, synthetic construct
  VARIANT 15: note = X can be R or Q
  VARIANT 16: note = X can be E or D
  VARIANT 18: note = X can be M or L
  VARIANT 38: note = X can be A or S
  VARIANT 44: note = X can be N or S
  VARIANT 52: note = X can be K or N
  VARIANT 57: note = X can be V or M
  VARIANT 69: note = X can be K or R
  VARIANT 79: note = X can be Q or E
  VARIANT 82: note = X can be V or I
  VARIANT 95: note = X can be H or R
  VARIANT 96: note = X can be Y or F
  VARIANT 105: note = X can be P or L
GQPREPQVYTLPPCXXEXTKNQVSLWCLVKGFYPSDIXVEWESXGQPENNYXTTPPXLDSDGSFFLYSXLTVDKSRWQXGNXFSCSVMHEALHNXXTQKSLSLSXGK

SEQ ID NO: 51 - protein, 107 aa, synthetic construct
  VARIANT 15: note = X can be R or Q
  VARIANT 16: note = X can be E or D
  VARIANT 18: note = X can be M or L
  VARIANT 38: note = X can be A or S
  VARIANT 44: note = X can be N or S
  VARIANT 52: note = X can be K or N
  VARIANT 57: note = X can be V or M
  VARIANT 69: note = X can be K or R
  VARIANT 79: note = X can be Q or E
  VARIANT 82: note = X can be V or I
  VARIANT 95: note = X can be H or R
  VARIANT 96: note = X can be Y or F
  VARIANT 105: note = X can be P or L
GQPREPQVCTLPPSXXEXTKNQVSLSCAVKGFYPSDIXVEWESXGQPENNYXTTPPXLDSDGSFFLVSXLTVDKSRWQXGNXFSCSVMHEALHNXXTQKSLSLSXGK

SEQ ID NO: 52 - protein, 107 aa, synthetic construct
GQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 107 aa, synthetic construct
GQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 54 - protein, 107 aa, synthetic construct
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 107 aa, synthetic construct
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 56 - protein, 107 aa, synthetic construct
GQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 107 aa, synthetic construct
GQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 58 - protein, 107 aa, synthetic construct
GQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 107 aa, synthetic construct
GQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 60 - protein, 107 aa, synthetic construct
GQPREPQVYTLPPSREEMTKNQVSLWCLVKGFYPSDISVEWESNGQPENNYKTTPPMLDSDGSFFLYSKLTVDKSRWQQGNVFSCSVMHEALHNHYTQKSLSLSPGK

SEQ ID NO: 61 - protein, 107 aa, synthetic construct
GQPREPQVYTLPPSREEMTKNQVSLSCAVKGFYPSDISVEWESNGQPENNYKTTPPMLDSDGSFFLVSKLTVDKSRWQQGNVFSCSVMHEALHNHYTQKSLSLSPGK

SEQ ID NO: 62 - protein, 107 aa, synthetic construct
GQPREPQVYTLPPCREEMTKNQVSLWCLVKGFYPSDISVEWESNGQPENNYKTTPPMLDSDGSFFLYSKLTVDKSRWQQGNVFSCSVMHEALHNHYTQKSLSLSPGK

SEQ ID NO: 63 - protein, 107 aa, synthetic construct
GQPREPQVCTLPPSREEMTKNQVSLSCAVKGFYPSDISVEWESNGQPENNYKTTPPMLDSDGSFFLVSKLTVDKSRWQQGNVFSCSVMHEALHNHYTQKSLSLSPGK

SEQ ID NO: 64 - protein, 106 aa, synthetic construct
GQPREPQVYTLPPSREEMTKNQVSLWCLVKGFYPSDIAVEWESSGQPENNYNTTPPMLDSDGSFFLYSKLTVDKSRWQQGNIFSCSVMHEALHNRFTQKSLSLSPG

SEQ ID NO: 65 - protein, 106 aa, synthetic construct
GQPREPQVYTLPPSREEMTKNQVSLSCAVKGFYPSDIAVEWESSGQPENNYNTTPPMLDSDGSFFLVSKLTVDKSRWQQGNIFSCSVMHEALHNRFTQKSLSLSPG

SEQ ID NO: 66 - protein, 106 aa, synthetic construct
GQPREPQVYTLPPCREEMTKNQVSLWCLVKGFYPSDIAVEWESSGQPENNYNTTPPMLDSDGSFFLYSKLTVDKSRWQQGNIFSCSVMHEALHNRFTQKSLSLSPG

SEQ ID NO: 67 - protein, 106 aa, synthetic construct
GQPREPQVCTLPPSREEMTKNQVSLSCAVKGFYPSDIAVEWESSGQPENNYNTTPPMLDSDGSFFLVSKLTVDKSRWQQGNIFSCSVMHEALHNRFTQKSLSLSPG

SEQ ID NO: 68 - protein, 107 aa, synthetic construct
GQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 69 - protein, 107 aa, synthetic construct
GQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 70 - protein, 107 aa, synthetic construct
GQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 71 - protein, 107 aa, synthetic construct
GQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 72 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 74 - protein, 236 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVEGFYPSDIAVEWESNGQPENNYKTTPPVLDSDGSFFLYSKLTVDKSRWQQGNVFSCSVMHEALHNHYTQKSLSLSPGKGGGGSEPEA

SEQ ID NO: 75 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 236 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDKLTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSEPEA

SEQ ID NO: 77 - protein, 218 aa, synthetic construct
QSVLTQPPSVSGAPGQRVTISCTGSSSNIGAGYGVHWYQQLPGTAPKLLIYGNTNRPSGVPDRFSGFKSGTSASLAITGLQAEDEADYYCQSYDSSLSGWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 449 aa, synthetic construct
QVQLVESGGGLVQPGGSLRLSCAASGFTFRSYAMSWVRQAPGKGLEWVSAISGRGDNTYYADSVKGRFTISRDNSKNTLYLQMNSLRAEDTAVYYCAKMTSNAFAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 79 - protein, 487 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSQSVLTQPPSVSGAPGQRVTISCTGSSSNIGAGYGVHWYQQLPGTAPKLLIYGNTNRPSGVPDRFSGFKSGTSASLAITGLQAEDEADYYCQSYDSSLSGWVFGGGTKLTVLGGGGSGGGGSGGGGSGGGSGQVQLVESGGGLVQPGGSLRLSCAASGFTFRSYAMSWVRQAPGKGLEWVSAISGRGDNTYYADSVKGRFTISRDNSKNTLYLQMNSLRAEDTAVYYCAKMTSNAFAFDYWGQGTLVTVSS

SEQ ID NO: 80 - protein, 218 aa, synthetic construct
QSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 459 aa, synthetic construct
QVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 82 - protein, 497 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSQSVLTQPPSVSAAPGQKVTISCSGSSSNIGNNYVSWYQQLPGTAPKLLIYGHTNRPAGVPDRFSGSKSGTSASLAISGFRSEDEADYYCAAWDDSLSGWVFGGGTKLTVLGGGGGSGGGGSGGGGSGGGSGQVQLLQSGAELKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSS

SEQ ID NO: 83 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 85 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 88 - protein, 236 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDKLTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSEPEA

SEQ ID NO: 89 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 236 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLWCLVEGFYPSDIAVEWESNGQPENNYKTTPPVLDSDGSFFLYSKLTVDKSRWQQGNVFSCSVMHEALHNHYTQKSLSLSPGKGGGGSEPEA

SEQ ID NO: 91 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 92 - protein, 475 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 93 - protein, 475 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK

SEQ ID NO: 94 - protein, 240 aa, synthetic construct
DKTHTCPPCPAPELLGGPSVFLFPPKPKDTLMISRTPEVTCVVVDVSHEDPEVKFNWYVDGVEVHNAKTKPREEQYNGGGSYRYRQGGGSSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 240 aa, synthetic construct
DKTHTCPPCPAPELLGGPSVFLFPPKPKDTLMISRTPEVTCVVVDVSHEDPEVKFNWYVDGVEVHNAKTKPREEQYNGGGSYRYRQGGGSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 96 - protein, 240 aa, synthetic construct
DKTHTCPPCPAPEAAGGPSVFLFPPKPKDTLMISRTPEVTCVVVDVSHEDPEVKFNWYVDGVEVHNAKTKPREEQYNGGGSYRYRQGGGSSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 240 aa, synthetic construct
DKTHTCPPCPAPEAAGGPSVFLFPPKPKDTLMISRTPEVTCVVVDVSHEDPEVKFNWYVDGVEVHNAKTKPREEQYNGGGSYRYRQGGGSSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 98 - protein, 239 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSYRYRQGGGS

SEQ ID NO: 99 - protein, 239 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGSYRYRQGGGS

SEQ ID NO: 100 - protein, 244 aa, synthetic construct
GLNDIFEAQKIEWHEGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 244 aa, synthetic construct
GLNDIFEAQKIEWHEGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 102 - protein, 244 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGLNDIFEAQKIEWHE

SEQ ID NO: 103 - protein, 244 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGLNDIFEAQKIEWHE

SEQ ID NO: 104 - protein, 4 aa, synthetic construct
  VARIANT >4: note = The entire sequence of amino acids 1-4 can be repeated two to six times
GGGS

SEQ ID NO: 105 - protein, 5 aa, synthetic construct
  VARIANT >5: note = The entire sequence of amino acids 1-5 can be repeated two to six times
GGGGS

SEQ ID NO: 106 - protein, 5 aa, synthetic construct
  VARIANT >4: note = The entire sequence of amino acids 1-4 can be repeated two to six times
GGGSG

SEQ ID NO: 107 - protein, 6 aa, synthetic construct
  VARIANT >4: note = The entire sequence of amino acids 1-4 can be repeated two to six times
GGGSGG

SEQ ID NO: 108 - protein, 7 aa, synthetic construct
  VARIANT >4: note = The entire sequence of amino acids 1-4 can be repeated two to six times
GGGSGGG

SEQ ID NO: 109 - protein, 6 aa, synthetic construct
  VARIANT >5: note = The entire sequence of amino acids 1-5 can be repeated two to six times
GGGGSG

SEQ ID NO: 110 - protein, 7 aa, synthetic construct
  VARIANT >5: note = The entire sequence of amino acids 1-5 can be repeated two to six times
GGGGSGG

SEQ ID NO: 111 - protein, 8 aa, synthetic construct
  VARIANT >5: note = The entire sequence of amino acids 1-5 can be repeated two to six times
GGGGSGGG

SEQ ID NO: 112 - protein, 20 aa, synthetic construct
GGGGSGGGGSGGGGSGGGGS

SEQ ID NO: 113 - protein, 30 aa, synthetic construct
GGGGSGGGGSGGGGSGGGGSGGGGSGGGGS

SEQ ID NO: 114 - protein, 5 aa, synthetic construct
GGGGS

SEQ ID NO: 115 - protein, 6 aa, synthetic construct
KKKKKK

SEQ ID NO: 116 - protein, 7 aa, synthetic construct
WLAQRPH

(19) United States
(12) Patent Application Publication
(10) Pub. No.: US 2025/0346675 A1
(43) Pub. Date: Nov. 13, 2025

(54) PAYLOAD-BEARING MULTISPECIFIC ANTIBODIES

(71) Applicant: Hoffmann-La Roche Inc., Little Falls, NJ (US)

(72) Inventors: Ulrich BRINKMANN, Weilheim (DE); Steffen Dickopf, Penzberg (DE)

(21) Appl. No.: 19/274,409

(22) Filed: Jul. 18, 2025

Related U.S. Application Data
(63) Continuation of application No. PCT/EP2024/051668, filed on Jan. 24, 2024.

(30) Foreign Application Priority Data
Jan. 25, 2023 (EP) 23153192.2

(51) Int. Cl. C07K 16/28; A61K 47/68; C07K 16/32

(52) U.S. Cl. CPC C07K 16/2863; A61K 47/6803; A61K 47/6849; A61K 47/6855; C07K 16/32; C07K 2317/524; C07K 2317/526; C07K 2317/53

(57) ABSTRACT

The present disclosure relates to a variant of ForCE technology (which is described e.g. in Dengl et al. 2020 and WO 2019/077092 A1), that can be employed for the production of payload-bearing molecules (such as antibody-drug conjugates), through combining functional (e.g. binding) entities with payload-coupled Fc molecules. The principle upon which the present disclosure is based is illustrated in the schematic of FIG. 1.

### CROSS-REFERENCE TO RELATED APPLICATIONS

This application is a continuation of International Application No. PCT/EP2024/051668, filed internationally on Jan. 24, 2024, which claims priority to and the benefit of European Patent Application No. 23153192.2, filed on Jan. 25, 2023, the contents of each of which are hereby incorporated herein in their entirety.

### REFERENCE TO AN ELECTRONIC SEQUENCE LISTING

The contents of the electronic sequence listing (256602005701SEQLIST.xml; Size: 159,834 bytes; and Date of Creation: Jul. 18, 2025) is herein incorporated by reference in its entirety.

### TECHNICAL FIELD

The present disclosure relates to the field of molecular biology, and in particular antigen-binding molecule technology.

### BACKGROUND

Antibody derivatives with attached payloads (e.g. Antibody Drug Conjugates, ADCs) serve as therapeutics, diagnostics and research tools. For these purposes, ADCs must retain preferentially uncompromised antigen binding, as well as desired payload functionalities and potencies (Nath et al. 2016; Akkapeddi et al. 2016). ADCs applied as drugs should additionally be of defined composition. This is quite a challenge, in particular for first generation conjugates which have their payloads attached via NHS chemistry to free amines (lysine residues) exposed on the surface of antibodies. Amine-exposing lysines that become modified by NHS are not only scattered among the antibody surfaces. They are in some instances also present in either CDRs or neighbouring Fv frameworks of antibodies. Conjugation on or near CDRs can compromise binding to the target antigen (Nath et al. 2016; Sadiki et al. 2020). Another hurdle for ADC discovery and screening approaches including functionality ranking is the difficulty of ensuring identical or at least functionally comparable conjugation of payloads to different antibodies.

Issues associated with random coupling can to an extent be addressed by application of site-directed conjugation. Most such approaches are based on the introduction of mutated residues that are targets for site-directed coupling. Examples are thiomabs, which carry exposed cysteines (Akkapeddi et al. 2016), antibodies with modified amino acids introduced during translation (e.g. via stop-codon suppression, (Beck et al. 2017; Patterson et al. 2014)), as well as antibodies with ‘tags’ that enable spontaneous or enzyme-based conjugations (e.g. inteins, SNAP, sortase, transglutaminase, (Beck et al. 2017; Hussain et al. 2021; Möhlmann et al. 2011; Steffen et al. 2017)). Production and scaling of these technologies is still complex and laborious in early project phases (or screens), as each conjugate must be produced individually and carefully analysed.

Further challenges remain. One major bottleneck is the fact that ADCs with desired functionalities are not the result of coupling a desired payload (e.g. a cytotoxin) to a well performing antibody at a position that can be addressed in a site specific manner. Instead, binding modules (antibodies, paratopes, formats) need to be compatible with payloads, and modes (linker composition), and positions of attachment must be compatible with binder as well as payload functionality. Stoichiometry (i.e. how many payloads are coupled to the antibody (drug to antibody ratio; DAR) at which positions) also affects ADC functionality, frequently also modulating biophysical and pharmacokinetic properties (Beck et al. 2017; Sun et al. 2017). The generation and identification/selection of optimal ADCs therefore requires the combination of different binders and formats or with various linker-payload modules at different positions, and in different DARs. Finding optimal ADCs therefore requires the assessment of matrices that combine those parameters. Even a small number of variables for each parameter (binder, format, linker, payload, position, DAR) results in large matrices. The production of comprehensive ADC matrices to cover that design space is tedious and a major hurdle in early development (e.g. for lead identification).

The use of a chain-exchange based Format Chain Exchange (ForCE) technology to generate large binder-format bispecific antibody (bsAb) matrices has recently been described, e.g. in Dengl et al. 2020 and WO 2019/077092 A1. ForCE is efficient and high throughput automation-compatible and produces combinations of bispecific antibodies in different formats from monospecific input molecules in vitro. Precursor molecules are applied as input modules that are half antibodies complemented with dummies, both associated with partially destabilised CH3 interfaces. Combining complementary precursors triggers exchange reactions that generate bsAb binder-binder-position-stoichiometry matrices (Dengl et al. 2020).

### SUMMARY

In a first aspect, the present disclosure provides a method for producing a polypeptide complex, comprising:
incubating:
   (1) a first polypeptide complex, comprising a first polypeptide comprising a CH3 domain and a second polypeptide comprising a CH3 domain;
   wherein the first polypeptide complex is formed by interaction between the first polypeptide and the second polypeptide, the interaction comprising association between the CH3 domain of the first polypeptide and the CH3 domain of the second polypeptide;
   wherein the CH3 domain of the first polypeptide comprises a knob modification, and the CH3 domain of the second polypeptide comprises a hole modification;
   wherein the CH3 domain of the first polypeptide or the CH3 domain of the second polypeptide comprises a destabilising modification for destabilising association between the CH3 domain of the first polypeptide and the CH3 domain of the second polypeptide;
   and wherein the first polypeptide and/or the second polypeptide further comprise a payload moiety;
and
   (2) a second polypeptide complex, comprising a third polypeptide comprising a CH3 domain and a fourth polypeptide comprising a CH3 domain;
   wherein the second polypeptide complex is formed by interaction between the third polypeptide and the fourth polypeptide, the interaction comprising association between the CH3 domain of the third polypeptide and the CH3 domain of the fourth polypeptide;
   wherein the CH3 domain of the third polypeptide comprises a knob modification, and the CH3 domain of the fourth polypeptide comprises a hole modification;
   wherein the CH3 domain of the third polypeptide or the CH3 domain of the fourth polypeptide comprises a destabilising modification for destabilising association between the CH3 domain of the third polypeptide and the CH3 domain of the fourth polypeptide;
to form a third polypeptide complex comprising the first polypeptide and the fourth polypeptide, and/or a fourth polypeptide complex comprising the second polypeptide and the third polypeptide; and
recovering the third polypeptide complex and/or the fourth polypeptide complex;
wherein the destabilising modification of the CH3 domain of the first polypeptide or the second polypeptide does not destabilise association between the first polypeptide and the fourth polypeptide, and does not destabilise association between the second polypeptide and the third polypeptide;
and wherein the destabilising modification of the CH3 domain of the third polypeptide or the fourth polypeptide does not destabilise association between the first polypeptide and the fourth polypeptide, and does not destabilise association between the second polypeptide and the third polypeptide.

In some embodiments, the destabilising modification of the CH3 domain of the first polypeptide or the second polypeptide stabilises association between the CH3 domain of the first polypeptide and the CH3 domain of the fourth polypeptide, and/or stabilises association between the CH3 domain of the second polypeptide and the CH3 domain of the third polypeptide.

In some embodiments, the destabilising modification of the CH3 domain of the third polypeptide or the fourth polypeptide stabilises association between the CH3 domain of the first polypeptide and the CH3 domain of the fourth polypeptide, and/or stabilises association between the CH3 domain of the second polypeptide and the CH3 domain of the third polypeptide.

In some embodiments, the first polypeptide comprises a payload moiety and the fourth polypeptide comprises a functional moiety, or the second polypeptide comprises a payload moiety and the third polypeptide comprises a functional moiety.

In some embodiments:
(a) the CH3 domain of the first polypeptide comprises 370E, and the CH3 domain of the fourth polypeptide comprises 357K; optionally wherein the CH3 domain of the second polypeptide comprises 357E, and the CH3 domain of the third polypeptide comprises 370K; or
(b) the CH3 domain of the first polypeptide comprises 370K, and the CH3 domain of the fourth polypeptide comprises 357E; optionally wherein the CH3 domain of the second polypeptide comprises 357K, and the CH3 domain of the third polypeptide comprises 370E.

In some embodiments:
(a) the CH3 domain of the first polypeptide comprises 366W and 370E, the CH3 domain of the second polypeptide comprises 407V, 366S, and 368A, the CH3 domain of the third polypeptide comprises 366W, and the CH3 domain of the fourth polypeptide comprises 407V, 366S, 368A and 357K; or
(b) the CH3 domain of the first polypeptide comprises 366W, 370E and 354C, the CH3 domain of the second polypeptide comprises 407V, 366S, and 368A, the CH3 domain of the third polypeptide comprises 366W, and the CH3 domain of the fourth polypeptide comprises 407V, 366S, 368A, 357K and 349C; or
(c) the CH3 domain of the first polypeptide has at least 70% amino acid sequence identity to SEQ ID NO:24, the CH3 domain of the second polypeptide has at least 70% amino acid sequence identity to SEQ ID NO:49, the CH3 domain of the third polypeptide has at least 70% amino acid sequence identity to SEQ ID NO:48, and the CH3 domain of the fourth polypeptide has at least 70% amino acid sequence identity to SEQ ID NO:25; or
(d) the CH3 domain of the first polypeptide has at least 70% amino acid sequence identity to SEQ ID NO:26, the CH3 domain of the second polypeptide has at least 70% amino acid sequence identity to SEQ ID NO:49, the CH3 domain of the third polypeptide has at least 70% amino acid sequence identity to SEQ ID NO:48, and the CH3 domain of the fourth polypeptide has at least 70% amino acid sequence identity to SEQ ID NO:27.

In some embodiments, the payload moiety is or comprises: a detectable moiety, a fluorescent moiety, a luminescent moiety, a radiopaque/contrast agent, a radiolabel, an immuno-detectable moiety, a moiety having a detectable activity, an enzymatic moiety, a drug moiety, or a cytotoxic moiety.

In some embodiments, the functional moiety is or comprises: a binding moiety, an antibody or a target-binding fragment or derivative thereof, a target-binding peptide/polypeptide, a target-binding nucleic acid, a detectable moiety, a fluorescent moiety, a luminescent moiety, a radiopaque/contrast agent, a radiolabel, an immuno-detectable moiety, a moiety having a detectable activity, an enzymatic moiety, a drug moiety or a cytotoxic moiety.

In some embodiments, the first polypeptide, the second polypeptide, the third polypeptide and/or the fourth polypeptide further comprise a CH2 domain and/or a hinge region.

The present disclosure also provides a polypeptide complex according to the third polypeptide complex or the fourth polypeptide complex, produced by the method according to the present disclosure.

The present disclosure also provides a polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide comprises a knob modification, and the CH3 domain of the second polypeptide comprises a hole modification;
wherein the CH3 domain of the first polypeptide comprises a destabilising modification, and wherein the CH3 domain of the second polypeptide comprises a destabilising modification;
wherein the destabilising modification of the CH3 domain of the first polypeptide does not destabilise association between the CH3 domain of the first polypeptide and the CH3 domain of the second polypeptide, and wherein the destabilising modification of the CH3 domain of the second polypeptide does not destabilise association between the CH3 domain of the first polypeptide and the CH3 domain of the second polypeptide; and
wherein the first polypeptide or the second polypeptide further comprises a payload moiety.

In some embodiments, the first polypeptide and/or the second polypeptide further comprise a functional moiety.

In some embodiments, the destabilising modification of the CH3 domain of the first polypeptide stabilises association between the CH3 domain of the first polypeptide and the CH3 domain of the second polypeptide; and/or wherein the destabilising modification of the CH3 domain of the second polypeptide stabilises association between the CH3 domain of the first polypeptide and the CH3 domain of the second polypeptide.

In some embodiments, the first polypeptide comprises a payload moiety and the second polypeptide comprises a functional moiety, or wherein the first polypeptide comprises a functional moiety and the second polypeptide comprises a payload moiety.

In some embodiments:
(a) the CH3 domain of the first polypeptide comprises 366W and 370E; and the CH3 domain of the second polypeptide comprises 407V, 366S, 368A and 357K; or
(b) the CH3 domain of the first polypeptide comprises 366W, 370E and 354C; and the CH3 domain of the second polypeptide comprises 407V, 366S, 368A, 357K and 349C; or
(c) the CH3 domain of the first polypeptide has at least 70% amino acid sequence identity to SEQ ID NO:24; and the CH3 domain of the second polypeptide has at least 70% amino acid sequence identity to SEQ ID NO:25; or
(d) the CH3 domain of the first polypeptide has at least 70% amino acid sequence identity to SEQ ID NO:26; and the CH3 domain of the second polypeptide has at least 70% amino acid sequence identity to SEQ ID NO:27.

In some embodiments, the payload moiety is or comprises: a detectable moiety, a fluorescent moiety, a luminescent moiety, a radiopaque/contrast agent, a radiolabel, an immuno-detectable moiety, a moiety having a detectable activity, an enzymatic moiety, a drug moiety, or a cytotoxic moiety.

In some embodiments, the functional moiety is or comprises: a binding moiety, an antibody or a target-binding fragment or derivative thereof, a target-binding peptide/polypeptide, a target-binding nucleic acid, a detectable moiety, a fluorescent moiety, a luminescent moiety, a radiopaque/contrast agent, a radiolabel, an immuno-detectable moiety, a moiety having a detectable activity, an enzymatic moiety, a drug moiety or a cytotoxic moiety.

In some embodiments, the first polypeptide and/or the second polypeptide further comprise a CH2 domain and/or a hinge region.

The present disclosure also provides a polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide comprises a knob modification, and the CH3 domain of the second polypeptide comprises a hole modification;
wherein the CH3 domain of the first polypeptide and/or the CH3 domain of the second polypeptide comprises a destabilising modification for destabilising association between the CH3 domain of the first polypeptide and the CH3 domain of the second polypeptide;
and wherein the first polypeptide and/or the second polypeptide further comprise a payload moiety.

In some embodiments:
(a) the CH3 domain of the first polypeptide comprises 370E, and the CH3 domain of the second polypeptide comprises 357E; or
(b) the CH3 domain of the first polypeptide comprises 370K, and the CH3 domain of the second polypeptide comprises 357K.

In some embodiments:
(a) the CH3 domain of the first polypeptide comprises 366W and 370E; and the CH3 domain of the second polypeptide comprises 407V, 366S, and 368A; or
(b) the CH3 domain of the first polypeptide comprises 366W; and the CH3 domain of the second polypeptide comprises 407V, 366S, 368A and 357K; or
(c) the CH3 domain of the first polypeptide comprises 366W, 370E and 354C; and the CH3 domain of the second polypeptide comprises 407V, 366S, and 368A; or
(d) the CH3 domain of the first polypeptide comprises 366W; and the CH3 domain of the second polypeptide comprises 407V, 366S, 368A, 357K and 349C; or
(e) the CH3 domain of the first polypeptide comprises 366W and 370E; and the CH3 domain of the second polypeptide comprises 407V, 366S, 368A and 349C; or
(f) the CH3 domain of the first polypeptide comprises 366W and 354C; and the CH3 domain of the second polypeptide comprises 407V, 366S, 368A, and 357K; or
(g) the CH3 domain of the first polypeptide has at least 70% amino acid sequence identity to SEQ ID NO:48; and the CH3 domain of the second polypeptide has at least 70% amino acid sequence identity to SEQ ID NO:25; or
(h) the CH3 domain of the first polypeptide has at least 70% amino acid sequence identity to SEQ ID NO:26; and the CH3 domain of the second polypeptide has at least 70% amino acid sequence identity to SEQ ID NO:49; or
(i) the CH3 domain of the first polypeptide has at least 70% amino acid sequence identity to SEQ ID NO:48; and the CH3 domain of the second polypeptide has at least 70% amino acid sequence identity to SEQ ID NO:27; or
(j) the CH3 domain of the first polypeptide has at least 70% amino acid sequence identity to SEQ ID NO:24; and the CH3 domain of the second polypeptide has at least 70% amino acid sequence identity to SEQ ID NO:51; or
(k) the CH3 domain of the first polypeptide has at least 70% amino acid sequence identity to SEQ ID NO:50; and the CH3 domain of the second polypeptide has at least 70% amino acid sequence identity to SEQ ID NO:25.

In some embodiments, the payload moiety is or comprises: a detectable moiety, a fluorescent moiety, a luminescent moiety, a radiopaque/contrast agent, a radiolabel, an immuno-detectable moiety, a moiety having a detectable activity, an enzymatic moiety, a drug moiety, or a cytotoxic moiety.

In some embodiments, the first polypeptide and/or the second polypeptide further comprise a CH2 domain and/or a hinge region.

### DESCRIPTION

The present disclosure relates to a variant of ForCE technology that can be employed for the production of payload-bearing molecules (such as ADCs), through combining functional (e.g. binding) entities with payload-coupled Fe molecules. This opens a robust, rapid and reliable route for generation of defined matrices of ADCs, connecting different binders in different formats at defined positions and stoichiometry via various linkers to payloads such as small molecules, peptides, nucleic acids and proteins.

The principle upon which the present disclosure is based is illustrated in the schematic of FIG. 1.

A first ‘donor’ precursor polypeptide complex comprises a first polypeptide and a second polypeptide, each comprising a CH3 domain. The CH3 domains of the first and second polypeptides comprise complementary modifications, promoting their association into polypeptide complexes (in the example of FIG. 1, ‘knob-into-hole’ modifications). The CH3 domains of the first and second polypeptides of the ‘donor’ precursor polypeptide complex further comprise one or more destabilising modifications, for destabilising interaction between the first and second polypeptides. One or both of the polypeptides further comprise a payload moiety.

A second ‘acceptor’ precursor polypeptide complex similarly comprises a first polypeptide and a second polypeptide, each comprising a CH3 domain. The CH3 domains of the first and second polypeptides comprise complementary modifications, promoting their association into polypeptide complexes (in the example of FIG. 1, ‘knob-into-hole’ modifications). The CH3 domains of the first and second polypeptides of the ‘acceptor’ precursor polypeptide complex further comprise one or more destabilising modifications, for destabilising interaction between the first and second polypeptides. One or both of the polypeptides may further comprise a functional moiety (in the example of FIG. 1, a Fab fragment).

A constituent polypeptide of a ‘donor’ precursor complex associates with a polypeptide of an ‘acceptor’ precursor complex with greater affinity than the affinity with which it associates with its interaction partner in the ‘donor’ precursor complex. Similarly, a constituent polypeptide of an ‘acceptor’ precursor complex associates with a polypeptide of a ‘donor’ precursor complex with greater affinity than the affinity with which it associates with its interaction partner in the ‘acceptor’ precursor complex. This is achieved through the destabilising modifications of the polypeptides of the donor and acceptor precursor complexes. The destabilising modification of a polypeptide of the ‘acceptor’ precursor polypeptide complex does not destabilise association between a polypeptide of the ‘acceptor’ precursor polypeptide complex and a polypeptide of the ‘donor’ precursor polypeptide complex. Similarly, the destabilising modification of a polypeptide of the ‘donor’ precursor polypeptide complex does not destabilise association between a polypeptide of the ‘donor’ precursor polypeptide complex and a polypeptide of the ‘acceptor’ precursor polypeptide complex.

The destabilising modification of a polypeptide of the ‘acceptor’ precursor polypeptide complex may stabilise association between a polypeptide of the ‘acceptor’ precursor polypeptide complex and a polypeptide of the ‘donor’ precursor polypeptide complex. Similarly, the destabilising modification of a polypeptide of the ‘donor’ precursor polypeptide complex may stabilise association between a polypeptide of the ‘donor’ precursor polypeptide complex and a polypeptide of the ‘acceptor’ precursor polypeptide complex. For example, as illustrated in FIG. 1, a destabilising modification of CH3 domain of a polypeptide of a ‘donor’ precursor complex may introduce an amino acid residue having a repulsive charge with respect to the charge of the amino acid residue with which it interacts in the other polypeptide of the ‘donor’ precursor complex, but this same amino acid residue may contribute to the formation of a salt bridge with an amino acid residue in the CH3 domain of a polypeptide of an ‘acceptor’ precursor complex. Similarly, a destabilising modification of CH3 domain of a polypeptide of an ‘acceptor’ precursor complex may introduce an amino acid residue having a repulsive charge with respect to the charge of the amino acid residue with which it interacts in the other polypeptide of the ‘acceptor’ precursor complex, but this same amino acid residue may contribute to the formation of a salt bridge with an amino acid residue in the CH3 domain of a polypeptide of a ‘donor’ precursor complex.

The modifications for promoting association between the constituent polypeptides of a ‘donor’ precursor complex, and the modifications for promoting association between the constituent polypeptides of an ‘acceptor’ precursor complex are preferably suitable for promoting association between a polypeptide of the ‘donor’ precursor complex, and a polypeptide of the ‘acceptor’ precursor complex. For example, as illustrated in FIG. 1, the CH3 domain of a polypeptide of a ‘donor’ precursor complex may comprise a knob modification, promoting association with the CH3 domain of a polypeptide of an ‘acceptor’ precursor complex comprising a hole modification. Similarly, the CH3 domain of a polypeptide of a ‘donor’ precursor complex may comprise a hole modification, promoting association with the CH3 domain of a polypeptide of an ‘acceptor’ precursor complex comprising a knob modification.

When the ‘donor’ and ‘acceptor’ precursor complexes are incubated with one another, polypeptide exchange between the ‘donor’ and ‘acceptor’ complexes results in the formation of two new complexes: (i) a final, payload-bearing complex, comprising a payload moiety-bearing polypeptide of the ‘donor complex’, and a polypeptide of the ‘acceptor complex’, optionally bearing a functional moiety (in the example of FIG. 1, a ‘defined, labelled antibody’); and (ii) a by-product ‘dummy’ complex, comprising the polypeptides of the ‘donor’ and ‘acceptor’ precursor complexes which are not comprised in the final, payload-bearing complex (in the example of FIG. 1, a ‘dummy dimer’).

### Polypeptide Complexes

Aspects of the present disclosure relate to polypeptide complexes. Herein, a ‘polypeptide complex’ refers to a complex formed by protein-protein interaction between two or more polypeptide monomers. Herein, a ‘polypeptide’ refers to a polymer chain of a plurality of amino acid monomers linked by peptide bonds.

Polypeptide complexes may be formed by non-covalent and/or covalent interaction between its constituent polypeptides. Non-covalent interactions include e.g. electrostatic interactions (e.g. ionic bonds, salt bridges) hydrogen bonds, Van der Waals forces and hydrophobic interactions. Covalent interactions include e.g. disulfide bonds. It will be appreciated that interactions forming polypeptide complexes involve amino acids/sequences of amino acids from different polypeptide monomers (i.e. inter-chain interactions).

In aspects and embodiments of the present disclosure, the constituent polypeptides of the polypeptide complexes comprise CH3 domains, and the polypeptide complexes are formed by interactions comprising association between the CH3 domains of the constituent polypeptides of the polypeptide complex. In some embodiments, the polypeptide complexes are formed by interaction comprising disulfide bonding between CH3 regions of the constituent polypeptides of the polypeptide complex.

In aspects and embodiments of the present disclosure, the constituent polypeptides of the polypeptide complexes comprise CH2 domains, and the polypeptide complexes are formed by interaction comprising association between the CH2 domains of the constituent polypeptides of the polypeptide complex.

In aspects and embodiments of the present disclosure, the constituent polypeptides of the polypeptide complexes comprise hinge regions, and the polypeptide complexes are formed by interaction comprising association between the hinge regions of the constituent polypeptides of the polypeptide complex. In some embodiments, the polypeptide complexes are formed by interaction comprising disulfide bonding between hinge regions of the constituent polypeptides of the polypeptide complex.

In some embodiments, constituent polypeptides of polypeptide complexes according to the present disclosure comprise a CH3 domain and a CH2 domain. In such embodiments, the constituent polypeptides of the polypeptide complexes may interact with one another through association between their CH3 and/or CH2 regions to form an Fc region. That is, in some embodiments, a polypeptide complex according to the present disclosure may be or comprise an Fc region.

As used herein, an ‘Fc region’ refers to a polypeptide complex formed by interaction between polypeptides each comprising a CH2 domain and a CH3 domain. In preferred embodiments, an Fc region may be a polypeptide complex formed by interaction between polypeptides each comprising the structure: N term-[ . . . ]-[CH2 domain]-[CH3 domain]-[ . . . ]-C term.

As used in representations of polypeptide structures herein, ‘[ . . . ]’ indicates the optional presence of further protein domain(s)/region(s). For example, in the structure of the final sentence of the preceding paragraph, further protein domain(s)/region(s) may optionally be present downstream of the CH3 domain, before the C terminus of the polypeptide. Furthermore, as used in representations of polypeptide structures herein ‘-’ indicates an optional linker sequence. For example, in the structure of the final sentence of the preceding paragraph, a linker sequence may optionally be provided between the CH2 domain and the CH3 domain.

Fc regions provide for interaction with Fc receptors and other molecules of the immune system to bring about functional effects. IgG Fc-mediated effector functions are reviewed e.g. in Jefferis et al., Immunol Rev 1998 163:59-76 (hereby incorporated by reference in its entirety), and are brought about through Fc-mediated recruitment and activation of immune cells (e.g. macrophages, dendritic cells, neutrophils, basophils, eosinophils, platelets, mast cells, NK cells and T cells) through interaction between the Fc region and Fc receptors expressed by the immune cells, recruitment of complement pathway components through binding of the Fc region to complement protein Clq, and consequent activation of the complement cascade. Fc-mediated functions include Fc receptor binding, antibody-dependent cellular cytotoxicity (ADCC), antibody-dependent cell-mediated phagocytosis (ADCP), complement-dependent cytotoxicity (CDC), formation of the membrane attack complex (MAC), cell degranulation, cytokine and/or chemokine production, and antigen processing and presentation.

Polypeptide complexes according to the present disclosure broadly fall into four classes: ‘donor’ precursor complexes, ‘acceptor’ precursor complexes, final payload-bearing complexes, and by-product ‘dummy’ complexes.

Precursor complexes are formed by interaction between polypeptides comprising CH3 domains, the interaction comprising association between the CH3 domains of the polypeptides. The CH3 domains of the polypeptides comprise modification for promoting their association, e.g. as described hereinbelow. In particular, the CH3 domains of polypeptides of precursor complexes may comprise paired ‘knob’ and ‘hole’ modifications, as described hereinbelow. Importantly, the polypeptides of precursor complexes also comprise modification to one or both of the CH3 domains, for destabilising association between the CH3 domains. The destabilisation of association conferred by the destabilising modification(s) should not be so great as to substantially prevent interaction between the polypeptides and thus formation of precursor complexes.

One or both polypeptides of a ‘donor’ precursor polypeptide complex further comprises a payload moiety. One or both polypeptides of an ‘acceptor’ precursor polypeptide complex may further comprise a functional moiety.

A constituent polypeptide of a ‘donor’ precursor complex associates with a polypeptide of an ‘acceptor’ precursor complex with greater affinity than the affinity with which it associates with its interaction partner in the ‘donor’ precursor complex. Similarly, a constituent polypeptide of an ‘acceptor’ precursor complex associates with a polypeptide of a ‘donor’ precursor complex with greater affinity than the affinity with which it associates with its interaction partner in the ‘acceptor’ precursor complex. This is achieved through the destabilising modifications of the polypeptides of the donor and acceptor precursor complexes. The destabilising modification of a polypeptide of the ‘acceptor’ precursor polypeptide complex does not destabilise association between a polypeptide of the ‘acceptor’ precursor polypeptide complex and a polypeptide of the ‘donor’ precursor polypeptide complex. Similarly, the destabilising modification of a polypeptide of the ‘donor’ precursor polypeptide complex does not destabilise association between a polypeptide of the ‘donor’ precursor polypeptide complex and a polypeptide of the ‘acceptor’ precursor polypeptide complex. The modifications for promoting association between the constituent polypeptides of a ‘donor’ precursor complex, and the modifications for promoting association between the constituent polypeptides of an ‘acceptor’ precursor complex are preferably suitable for promoting association between a polypeptide of the ‘donor’ precursor complex, and a polypeptide of the ‘acceptor’ precursor complex.

Final payload-bearing complexes are formed by interaction between polypeptides comprising CH3 domains, the interaction comprising association between the CH3 domains of the polypeptides. A polypeptide of the complex further comprises a payload moiety. The CH3 domains of the polypeptides comprise modification for promoting their association, e.g. as described hereinbelow. In particular, the CH3 domains of polypeptides of precursor complexes may comprise paired ‘knob’ and ‘hole’ modifications, as described hereinbelow. Association between the constituent polypeptides of the final payload-bearing complexes is stronger than association between the constituent polypeptides of the precursor complexes described above. That is, final payload-bearing complexes have greater stability (e.g. lower propensity to disassociate) as compared to precursor complexes. In some embodiments, the polypeptides of final payload-bearing complexes comprise modification to one or both of the CH3 domains, for stabilising association between the CH3 domains. In some embodiments, a modification that is a destabilising modification of a constituent polypeptide of a precursor complex is a modification that stabilises association between the CH3 domains of the polypeptides of a final payload-bearing complex.

By-product ‘dummy’ complexes are similarly formed by interaction between polypeptides comprising CH3 domains, the interaction comprising association between the CH3 domains of the polypeptides. The CH3 domains of the polypeptides comprise modification for promoting their association, e.g. as described hereinbelow. In particular, the CH3 domains of polypeptides of precursor complexes may comprise paired ‘knob’ and ‘hole’ modifications, as described hereinbelow. Association between the constituent polypeptides of the by-product ‘dummy’ complexes is stronger than association between the constituent polypeptides of the precursor complexes described above. That is, by-product ‘dummy’ complexes have greater stability (e.g. lower propensity to disassociate) as compared to precursor complexes. In some embodiments, the polypeptides of by-product ‘dummy’ complexes comprise modification to one or both of the CH3 domains, for stabilising association between the CH3 domains. In some embodiments, a modification that is a destabilising modification of a constituent polypeptide of a precursor complex is a modification that stabilises association between the CH3 domains of the polypeptides of a by-product ‘dummy’ complex.

It will be appreciated that a final payload-bearing complex according to the present disclosure is more stable/has a lower propensity to dissociate/is formed by polypeptides interacting with greater affinity, as compared to a precursor polypeptide complex according to the present disclosure (e.g. a ‘donor’ or ‘acceptor’ polypeptide complex).

Similarly, a by-product ‘dummy’ complex according to the present disclosure is more stable/has a lower propensity to dissociate/is formed by polypeptides interacting with greater affinity, as compared to a precursor polypeptide complex according to the present disclosure (e.g. a ‘donor’ or ‘acceptor’ polypeptide complex).

### CH3 Domains

Herein, a ‘CH3 domain’ refers to an amino acid sequence corresponding to the CH3 domain of an immunoglobulin (Ig). The CH3 domain is the region of an Ig formed by positions 341 to 447 of the immunoglobulin constant domain, according to the EU numbering system described in Edelman et al., Proc. Natl. Acad. Sci. USA (1969) 63 (1): 78-85.

In some embodiments, the CH3 domain corresponds to or is derived from the CH3 domain of an Ig from a mammal (e.g. a therian, placental, epitherian, preptotheria, archontan, primate (rhesus, cynomolgous, non-human primate or human)). In some embodiments, the CH3 domain corresponds to or is derived from the CH3 domain of an Ig from a human.

In some embodiments, the CH3 domain corresponds to or is derived from the CH3 domain of an IgG (e.g. IgG1, IgG2, IgG3, IgG4), IgA (e.g. IgA1, IgA2), IgD, IgE or IgM.

In some embodiments, the CH3 domain corresponds to or is derived from the CH3 domain of a human IgG (e.g. hIgG1, hIgG2, hIgG3, hIgG4), hIgA (e.g. hIgA1, hIgA2), hIgD, hIgE or hIgM. In some embodiments, the CH3 domain corresponds to or is derived from the CH3 domain of a human IgG1 allotype (e.g. G1 ml, G1m2, G1m3 or G1m17).

The CH3 domain of the Glml allotype of human IgG1 is formed by positions 224 to 330 of UniProt P01857-1, v1, and has the amino acid sequence shown in SEQ ID NO:1. The CH3 domain of the G1m3 allotype of human IgG1 is shown in SEQ ID NO:2. The CH3 domain of human IgG2 is formed by positions 220 to 326 of UniProt P01859-1, v2, and has the amino acid sequence shown in SEQ ID NO:3. The CH3 domain of human IgG3 is formed by positions 271 to 376 of UniProt P01860-1, v2, and has the amino acid sequence shown in SEQ ID NO:4. The CH3 domain of human IgG4 is formed by positions 221 to 327 of UniProt P01861-1, v1, and has the amino acid sequence shown in SEQ ID NO:5. The third Ig-like region of human IgA1 is formed by positions 228 to 330 of UniProt P01876-1, v2, and has the amino acid sequence shown in SEQ ID NO:6. The third Ig-like region of human IgA2 is formed by positions 215 to 317 of UniProt P01877-1, v4, and has the amino acid sequence shown in SEQ ID NO:7. The third Ig-like region of human IgD is formed by positions 267 to 373 of UniProt P01880-1, v3, and has the amino acid sequence shown in SEQ ID NO:8. The third Ig-like region of human IgE is formed by positions 214 to 318 of UniProt P01854-1, v1, and has the amino acid sequence shown in SEQ ID NO:9. The CH3 domain of human IgM is formed by positions 218 to 323 of UniProt P01871-1, v4, and has the amino acid sequence shown in SEQ ID NO:10.

Herein, an amino acid sequence which ‘corresponds’ to a specified domain/region of a reference polypeptide, or reference amino acid sequence, has at least 60%, e.g. one of at least ≥65%, ≥70%, ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to the amino acid sequence of the reference domain/region or the reference amino acid sequence. An amino acid sequence which ‘corresponds’ to a specified domain/region of a reference polypeptide or reference amino acid sequence can be identified by sequence alignment of the subject sequence to the reference sequence, e.g. using sequence alignment software such as ClustalOmega (Söding, J. 2005, Bioinformatics 21, 951-960).

An amino acid sequence which is ‘derived from’ a specified domain/region of a reference polypeptide, or reference amino acid sequence, has at least 60%, e.g. one of at least ≥65%, ≥70%, ≥75%, ≥80%, ≥85%, ≥90%, ≥91%, ≥92%, ≥93%, ≥94%, ≥95%, ≥96%, ≥97%, ≥98% ≥99% or 100% amino acid sequence identity to the amino acid sequence of the reference domain/region or the reference amino acid sequence. In some embodiments, an amino acid sequence which is derived from a specified domain/region of a reference polypeptide, or reference amino acid sequence, has at least 60%, e.g. one of at least ≥65%, ≥70%, ≥75%, ≥80%, ≥85%, ≥90%, ≥91%, ≥92%, ≥93%, ≥94%, ≥95%, ≥96%, ≥97%, ≥98% or ≥99% amino acid sequence to the amino acid sequence of the reference domain/region or the reference amino acid sequence, and is non-identical to the amino acid sequence of the reference domain/region or the reference amino acid sequence. In some embodiments, an amino acid sequence which is derived from a specified domain/region of a reference polypeptide, or reference amino acid sequence, comprises one or more (e.g. 1, 2, 3, 4, 5, 7, 8, 10 or more) differences relative to the amino acid sequence of the reference domain/region, or the reference amino acid sequence.

By way of illustration, the CH3 domain of human IgG2 is formed by positions 220 to 326 of UniProt P01859-1, v2, and has the amino acid sequence shown in SEQ ID NO:3. It will be appreciated that positions 220 to 326 of UniProt P01859-1, v2 correspond to positions 224 to 330 of UniProt P01857-1, v1.

In some embodiments, a CH3 domain according to the present disclosure comprises, or consists of, an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:1, 2, 3, 4, 5, 6, 7, 8, 9 or 10.

In some embodiments, a CH3 domain comprises, or consists of, an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:1, 2, 3, 4 or 5.

In some embodiments, a CH3 domain comprises, or consists of, an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:1.

In some embodiments, a CH3 domain comprises, or consists of, an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to one of SEQ ID NOs: 24 to 143.

In some embodiments, a CH3 domain comprises, or consists of, an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to one of SEQ ID NOs: 24 to 119.

As explained hereinbelow, in various aspects and embodiments according to the present disclosure CH3 domains comprise one or more modifications, e.g. modifications influencing association between CH3 domains. Where a CH3 domain described herein comprises a modification and moreover comprises or consists of an amino acid sequence within a specified threshold percent amino acid sequence identity to a reference amino acid sequence, it will be appreciated that any variation relative to the reference amino acid sequence is confined to positions of the reference sequence other than the modified position(s). By way of illustration, in the example of a CH3 domain comprising a destabilising modification according to the present disclosure and comprising an amino acid sequence having at least 70% amino acid sequence identity to SEQ ID NO:1, the CH3 domain necessarily comprises the destablising modification, and so the up to 30% variation from the amino acid sequence of SEQ ID NO:1 is confined to positions other than the modified position(s). By way of further illustration, in the example of a CH3 domain comprising 366W and 370E and comprising an amino acid sequence having at least 70% amino acid sequence identity to SEQ ID NO: 24, the up to 30% variation from the amino acid sequence of SEQ ID NO:24 is confined to positions of the CH3 domain sequence other than positions 366 and 370, which are necessarily 366W and 370E.

### Modifications to CH3 Domains Influencing Association Between CH3 Domains

CH3 domains according to the present disclosure comprise modification to promote association with another CH3 domain, and may also comprise modification to destabilise association with certain CH3 domains.

In some aspects and embodiments, a CH3 domain of the present disclosure comprises modification to promote association with another a CH3 domain. In some embodiments, a CH3 domain comprises modification to promote heteromerisation, i.e. association between non-identical CH3 domains.

Modifications to CH3 domains to promote association with another CH3 domain are known in the art, and include the paired modifications to CH3 regions (to be associated with one another) described in Ha et al., Front. Immunol (2016) 7:394 (hereby incorporated by reference in its entirety), particularly in Table 1 thereof. Such modifications include modifications of KiH, KiHs-s, HA-TF, ZW1, 7.8.60, DD-KK, EW-RVT, EW-RVTs-s, SEED and A107 technologies.

In aspects and embodiments of the present disclosure, interaction between constituent polypeptides of polypeptide complexes described herein is potentiated through ‘knob-into-hole’ technology. Knob-into-hole (or ‘KiH’) technology is described e.g. in WO 96/027011, Ridgway, J. B., et al., Protein Eng. 9 (1996) 617-621, Merchant, A. M., et al., Nat. Biotechnol. 16 (1998) 677-681, U.S. Pat. No. 7,695,936 and Carter, J. Immunol. Meth. (2001) 248, 7-15, all of which are hereby incorporated by reference in their entirety.

Heterodimerisation between CH3 domain-bearing polypeptides is promoted through modification of their interaction surfaces to provide complementary ‘knob’ and ‘hole’ modifications in the amino acid sequences of the CH3 domains of the polypeptides. The ‘knob’ and ‘hole’ modifications are positioned within the respective CH3 domains so that the ‘knob’ can be positioned in the ‘hole’ in order to promote heterodimerisation (and inhibit homodimerisation) of the polypeptides and/or stabilise heterodimers. Knobs are constructed by substituting amino acids having small side chains with those having larger side chains (e.g. tyrosine or tryptophan). Holes are created by substituting amino acids having large side chains with those having smaller side chains (e.g. valine, alanine, serine or threonine).

An additional interchain disulfide bridge between the CH3 domains may also be introduced (as described in Merchant, A. M., et al., Nature Biotech. 16 (1998) 677-681), e.g. through introduction of a cysteine residue at position 354 of the CH3 domain having a ‘knob’ modification, and introduction of a cysteine residue at position 349 of the CH3 domain having a ‘hole’ modification. Knob modifications further comprising modification to introduce a cysteine residue for the formation of an interchain disulfide bridge may be referred to as ‘knob-cys’ modifications, and similarly hole modifications further comprising modification to introduce a cysteine residue for the formation of an interchain disulfide bridge may be referred to as ‘hole-cys’ modifications.

As used herein, a ‘modification’ refers to a difference relative to a reference amino acid sequence. A reference amino acid sequence may be the amino acid sequence encoded by the most common nucleotide sequence of the gene encoding the relevant protein.

By way of illustration, in embodiments herein, certain CH3 domains comprise a knob modification, which may comprise modification at position 366 of the CH3 domain (unless otherwise stated, numbering of positions or substitutions in CH3 regions herein is according to the EU numbering system described in Edelman et al., Proc. Natl. Acad. Sci. USA (1969) 63 (1): 78-85). The modification may be to provide a tryptophan residue at position 366, which in the canonical sequences for human IgG1/IgG2/IgG3/IgG4 is a threonine residue.

In embodiments herein (and also in the art more generally), a ‘modification’ may also be referred to as a ‘substitution’ or a ‘mutation’.

A modification typically comprises substitution of an amino acid residue with a non-identical ‘replacement’ amino acid residue. A replacement amino acid residue of a modification according to the present disclosure may be a naturally-occurring amino acid residue (i.e. encoded by the genetic code) which is non-identical to the amino acid residue at the relevant position of the amino acid sequence prior to modification, selected from: alanine (Ala), arginine (Arg), asparagine (Asn), aspartic acid (Asp), cysteine (Cys), glutamine (Gln), glutamic acid (Glu), glycine (Gly), histidine (His), isoleucine (Ile): leucine (Leu), lysine (Lys), methionine (Met), phenylalanine (Phe), proline (Pro), serine (Ser), threonine (Thr), tryptophan (Trp), tyrosine (Tyr), and valine (Val). In some embodiments, a replacement amino acid residue of a modification may be a non-naturally occurring amino acid residue-i .e. an amino acid residue other than those recited in the preceding sentence. Examples of non-naturally occurring amino acid residues include norleucine, ornithine, norvaline, homoserine, aib, and other amino acid residue analogues such as those described in Ellman, et al., Meth. Enzym. 202 (1991) 301-336.

The amino acid employed is optionally in each case the L-form. The term “positively-charged” or “negatively-charged” amino acid refers to the amino acid side-chain charge at pH 7.4. Amino acids may be grouped according to common side-chain properties:
(1) hydrophobic: Norleucine, Met, Ala, Val, Leu, Ile, Trp, Tyr, Phe;
(2) neutral hydrophilic: Cys, Ser, Thr, Asn, Gln;
(3) acidic or negatively charged: Asp, Glu;
(4) basic or positively charged: His, Lys, Arg;
(5) residues that influence chain orientation: Gly, Pro.

| | Side-chain | Side-chain |
|---|---|---|
| Amino Acid | polarity | charge (pH 7.4) |
| Alanine | nonpolar | neutral |
| Arginine | basic polar | positive |
| Asparagine | polar | neutral |
| Aspartic acid | acidic polar | negative |
| Cysteine | nonpolar | neutral |
| Glutamic acid | acidic polar | negative |
| Glutamine | polar | neutral |
| Glycine | nonpolar | neutral |
| Histidine | basic polar | positive (10%) |
| | | neutral (90%) |
| Isoleucine | nonpolar | neutral |
| Leucine | nonpolar | neutral |
| Lysine | basic polar | positive |
| Methionine | nonpolar | neutral |
| Phenylalanine | nonpolar | neutral |
| Proline | nonpolar | neutral |
| Serine | polar | neutral |
| Threonine | polar | neutral |
| Tryptophan | nonpolar | neutral |
| Tyrosine | polar | neutral |
| Valine | nonpolar | neutral |

In some embodiments, a hydrophobic amino acid is selected from Norleucine, Met, Ala, Val, Leu, Ile, Trp, Tyr, and Phe. In some embodiments, a hydrophobic amino acid is selected from Ala, Val, Leu, Ile and Tyr. In some embodiments, a hydrophobic amino acid is Val, Leu, or Ile. In some embodiments, a hydrophobic amino acid is Leu or Ile. In some embodiments, a hydrophobic amino acid is Leu. In some embodiments, a hydrophobic amino acid is Tyr. In some embodiments, a hydrophobic amino acid is Phe.

In some embodiments, a positively charged amino acid is His, Lys, or Arg. In some embodiments, a positively charged amino acid is Lys, or Arg. In some embodiments, a positively charged amino acid is Lys.

In some embodiments, a negatively charged amino acid is Asp or Glu. In some embodiments, a negatively charged amino acid is Asp. In some embodiments, a negatively charged amino acid is Glu.

In embodiments and aspects of the present disclosure, CH3 domains—e.g. CH3 domains of constituent polypeptides of polypeptide complexes according to the present disclosure-comprise paired CH3 domain ‘KiH’ or ‘KiHs-s’ modifications.

In some embodiments, a CH3 domain comprising a knob modification comprises a tryptophan or tyrosine residue at position 366 (i.e. 366W or 366Y). In some embodiments, the knob modification is or comprises T366W or T366Y.

In some embodiments, a CH3 domain comprising a knob modification comprises 366W. In some embodiments, the knob modification is or comprises T366W.

Herein, when reference is made to a position of a immunoglobulin constant region (e.g. according to Eu numbering), the corresponding position in homologous sequences to the constant region sequence of human IgG1 (G1 ml allotype) are also contemplated. Corresponding positions to those identified in the CH3 domain of human IgG1 (G1 ml allotype) can be identified by sequence alignment, which can be performed e.g. using sequence alignment software such as ClustalOmega (Söding, J. 2005, Bioinformatics 21, 951-960).

By way of illustration, 366T in human IgG1 (position 26 of SEQ ID NO:1) corresponds to position 26 of SEQ ID NO:2 (hIgG1 G1m3 allotype), position 26 of SEQ ID NO:3 (hIgG2), position 26 of SEQ ID NO:4 (hIgG3), and position 26 of SEQ ID NO:5 (hIgG4).

Moreover, where a modification is indicated herein by reference to a reference amino acid of the constant region human IgG1 (G1 ml allotype), it will be appreciated that when such modification is provided in a homologous protein/domain thereof, substitution of the equivalent position in that sequence is intended. It is the amino acid residue at the relevant position following modification that is important. By way of illustration, human IgG1 (G1 m1) comprises D at position 356, while human IgG1 (G1m3) comprises E at position 356. ‘A CH3 domain comprising D356K’ of course encompasses a CH3 domain having the sequence of SEQ ID NO:1 and comprising the amino acid substitution D356K, but also encompasses e.g. a CH3 domain having the sequence of SEQ ID NO:2 and comprising the amino acid substitution E356K.

In some embodiments, a CH3 domain comprising a hole modification comprises 407V, 407A, 407S or 407T; 366S, 366V or 366A; and 368A, 368V, 368S or 368T. In some embodiments, the hole modification is or comprises Y407V, Y407A, Y407S or Y407T; T366S, T366V or T366A; and L368A, L368V, L368S or L368T.

In some embodiments, a CH3 domain comprising a hole modification comprises 407V, 366S and 368A. In some embodiments, the hole modification is or comprises Y407V, T366S, and L368A.

In aspects and embodiments of the present disclosure, CH3 domains of polypeptides of polypeptide complexes of the present disclosure comprise modification for the formation of an interchain disulfide bond (i.e. between the polypeptides). Such modification may comprise the introduction of one or more cysteine residues into one or both of the CH3 domains of the constituent polypeptides of a polypeptide complex of the present disclosure. More particularly, such modification may have the result that the CH3:CH3 interface formed between the CH3 domains of polypeptides of polypeptide complexes of the present disclosure comprises a disulfide bond, formed between cysteine residues (one from each polypeptide).

In some embodiments, a polypeptide complex according to the present disclosure (e.g. a final, payload-bearing polypeptide complex) comprises: (i) a polypeptide having a CH3 domain comprising the modification of one of rows 1 to 6 of column A of Table I; and (ii) a polypeptide having a CH3 domain comprising the modification of one of rows 1 to 6 of column B of Table I:

TABLE I

| | Column A | Column B |
|---|---|---|
| 1 | D399C | K392C |
| 2 | Y349C | S354C |
| 3 | Y349C | D356C |
| 4 | Y349C | E357C |
| 5 | L351C | S354C |
| 6 | T394C | V397C |

In preferred embodiments, for a given polypeptide complex (e.g. a final, payload-bearing polypeptide complex), the modification of the CH3 domain comprising a modification from column A and the modification of the CH3 domain from column B are selected from the same row of Table I. For example, in some embodiments, a polypeptide complex according to the present disclosure comprises: (i) a polypeptide having a CH3 domain comprising Y349C (row 2 of column A); and (ii) a polypeptide having a CH3 domain comprising S354C (row 2 of column B).

In some embodiments, a polypeptide according to the present disclosure (e.g. a precursor polypeptide complex) comprises modification introducing one or more cysteine residues into only one of the CH3 domains of the polypeptides of the polypeptide complex. Such modification may introduce a cysteine residue which does not participate in the formation of a disulfide bond in the polypeptide complex. Such modification may introduce a cysteine residue which does not participate in the formation of an interchain disulfide bond in a precursor polypeptide complex, but which does participate in the formation of a interchain disulfide bond in a final, payload-bearing polypeptide complex of the present disclosure.

By way of illustration, a final, payload-bearing polypeptide complex of the present disclosure (C) may be formed by polypeptide chain exchange between a first precursor polypeptide complex (A), and a second precursor polypeptide complex (B); wherein (A) comprises: (i) a first polypeptide comprising 349C, and (ii) a second polypeptide comprising 354S; wherein (B) comprises: (i) a first polypeptide comprising 349Y, and (ii) a second polypeptide comprising 354C; and wherein (C) comprises the first polypeptide of (A) (i.e. comprising 349C), and the second polypeptide of (B) (i.e. comprising 354C).

Accordingly, in some embodiments, a polypeptide complex according to the present disclosure (e.g. a precursor polypeptide complex) comprises: (i) a polypeptide having a CH3 domain comprising the modification of one of rows 1 to 6 of column A Table I; and (ii) a polypeptide having a CH3 domain lacking the modification of one of rows 1 to 6 of column B of Table I; or comprises: (i) a polypeptide having a CH3 domain lacking the modification of one of rows 1 to 6 of column A of Table I; and (ii) a polypeptide having a CH3 domain comprising the modification of one of rows 1 to 6 of column B of Table I (in preferred embodiments, for a given polypeptide complex, the modification of the CH3 domain from column A and the modification of the CH3 domain from column B are selected from the same row of Table I).

In some embodiments, a CH3 domain comprising a knob modification further comprises 354C. In some embodiments, the CH3 domain comprising a knob modification further comprises the modification S354C.

In some embodiments, a CH3 domain comprising a knob modification comprises 366W or 366Y; and 354C. In some embodiments, the knob modification is or comprises T366W or T366Y; and S354C.

In some embodiments, a CH3 domain comprising a knob modification comprises 366W and 354C. In some embodiments, the knob modification is or comprises T366W and S354C.

In some embodiments, a CH3 domain comprising a hole modification further comprises 349C. In some embodiments, the CH3 domain comprising a hole modification further comprises the modification Y349C.

In some embodiments, a CH3 domain comprising a hole modification comprises 407V, 407A, 407S or 407T; 366S, 366V or 366A; 368A, 368V, 368S or 368T; and 349C. In some embodiments, the hole modification is or comprises Y407V, Y407A, Y407S or Y407T; T366S, T366V or T366A; L368A, L368V, L368S or L368T; and Y349C.

In some embodiments, a CH3 domain comprising a hole modification comprises 407V, 366S, 368A and 354C. In some embodiments, the hole modification is or comprises Y407V, T366S, L368A and Y354C.

In some embodiments, a polypeptide complex according to the present disclosure comprises: (i) a first polypeptide having a CH3 domain comprising a knob modification, and (ii) a second polypeptide having a CH3 domain comprising a hole modification.

In some embodiments, a polypeptide complex according to the present disclosure comprises: (i) a first polypeptide having a CH3 domain comprising 366W, and (ii) a second polypeptide having a CH3 domain comprising 407V, 366S and 368A.

In some embodiments, a polypeptide complex according to the present disclosure (e.g. a final, payload-bearing polypeptide complex) comprises: (i) a first polypeptide having a CH3 domain comprising 366W and 354C, and (ii) a second polypeptide having a CH3 domain comprising 407V, 366S, 368A and 349C.

In some embodiments, a polypeptide complex according to the present disclosure (e.g. a precursor polypeptide complex) comprises: (i) a first polypeptide having a CH3 domain comprising 366W and 354C, and (ii) a second polypeptide having a CH3 domain comprising 407V, 366S, 368A and 349Y.

In some embodiments, a polypeptide complex according to the present disclosure (e.g. a precursor polypeptide complex) comprises: (i) a first polypeptide having a CH3 domain comprising 366W and 354S, and (ii) a second polypeptide having a CH3 domain comprising 407V, 366S, 368A and 349C.

In embodiments and aspects of the present disclosure, CH3 domains—e.g. CH3 domains of constituent polypeptides of polypeptide complexes according to the present disclosure-comprise paired CH3 domain ‘DD-KK’ modifications described in e.g. in U.S. Pat. No. 8,592,562 B2. In some embodiments, a CH3 domain comprises 392D and 409D. In some embodiments, a CH3 domain comprises 356K and 399K. In some embodiments, a polypeptide complex according to the present disclosure comprises: (i) a first polypeptide having a CH3 domain comprising 392D and 409D, and (ii) a second polypeptide having a CH3 domain comprising 356K and 399K.

In embodiments and aspects of the present disclosure, CH3 domains—e.g. CH3 domains of constituent polypeptides of polypeptide complexes according to the present disclosure-comprise paired CH3 domain ‘Duobody’ modifications described e.g. in Labrijn et al., Proc. Natl. Acad. Sci. USA. (2013) 110 (13): 5145-50. In some embodiments, a CH3 domain comprises 409R. In some embodiments, a CH3 domain comprises 405L. In some embodiments, a polypeptide complex according to the present disclosure comprises: (i) a first polypeptide having a CH3 domain comprising 409R, and (ii) a second polypeptide having a CH3 domain comprising 405L.

In embodiments and aspects of the present disclosure, CH3 domains—e.g. CH3 domains of constituent polypeptides of polypeptide complexes according to the present disclosure-comprise paired CH3 domain ‘EEE-RRR’ modifications described e.g. in Strop et al., J. Mol. Biol. (2012) 420 (3): 204-19. In some embodiments, a CH3 domain comprises 368E. In some embodiments, a CH3 domain comprises 409R. In some embodiments, a polypeptide complex according to the present disclosure comprises: (i) a first polypeptide having a CH3 domain comprising 368E, and (ii) a second polypeptide having a CH3 domain comprising 409R.

In embodiments and aspects of the present disclosure, CH3 domains—e.g. CH3 domains of constituent polypeptides of polypeptide complexes according to the present disclosure-comprise paired CH3 domain ‘EW-RVT’ modifications described e.g. in Choi et al., Mol. Cancer Ther. (2013) 12 (12): 2748-59. In some embodiments, a CH3 domain comprises 360E and 409W. In some embodiments, a CH3 domain comprises 347R, 399V and 405T. In some embodiments, a polypeptide complex according to the present disclosure comprises: (i) a first polypeptide having a CH3 domain comprising 360E and 409W, and (ii) a second polypeptide having a CH3 domain comprising 347R, 399V and 405T.

In embodiments and aspects of the present disclosure, CH3 domains—e.g. CH3 domains of constituent polypeptides of polypeptide complexes according to the present disclosure-comprise paired CH3 domain modifications described in Moore et al., MAbs (2011) 3 (6): 546-57. In some embodiments, a CH3 domain comprises 364H and 405A. In some embodiments, a CH3 domain comprises 349T and 394F. In some embodiments, a polypeptide complex according to the present disclosure comprises: (i) a first polypeptide having a CH3 domain comprising 364H and 405A, and (ii) a second polypeptide having a CH3 domain comprising 349T and 394F.

In embodiments and aspects of the present disclosure, CH3 domains—e.g. CH3 domains of constituent polypeptides of polypeptide complexes according to the present disclosure-comprise paired CH3 domain modifications described in Von Kreudenstein et al., MAbs (2013) 5 (5): 646-54. In some embodiments, a CH3 domain comprises 350V, 351Y, 405A and 407V. In some embodiments, a CH3 domain comprises 350V, 366L, 392L and 394W. In some embodiments, a polypeptide complex according to the present disclosure comprises: (i) a first polypeptide having a CH3 domain comprising 350V, 351Y, 405A and 407V, and (ii) a second polypeptide having a CH3 domain comprising 350V, 366L, 392L and 394W.

In embodiments and aspects of the present disclosure, CH3 domains—e.g. CH3 domains of constituent polypeptides of polypeptide complexes according to the present disclosure-comprise paired CH3 domain modifications described in Leaver-Fay et al., Structure (2016) 24 (4): 641-51, In some embodiments, a CH3 domain comprises 360D, 399M and 407A. In some embodiments, a CH3 domain comprises 345R, 347R, 366V and 409V. In some embodiments, a polypeptide complex according to the present disclosure comprises: (i) a first polypeptide having a CH3 domain comprising 360D, 399M and 407A, and (ii) a second polypeptide having a CH3 domain comprising 345R, 347R, 366V and 409V.

In embodiments and aspects of the present disclosure, CH3 domains—e.g. CH3 domains of constituent polypeptides of polypeptide complexes according to the present disclosure-comprise paired CH3 domain modifications described in Choi et al., PLOS One (2015) 10(12): e0145349. In some embodiments, a CH3 domain comprises 370E and 409W. In some embodiments, a CH3 domain comprises 357N, 399V and 405T. In some embodiments, a polypeptide complex according to the present disclosure comprises: (i) a first polypeptide having a CH3 domain comprising 370E and 409W, and (ii) a second polypeptide having a CH3 domain comprising 357N, 399V and 405T.

In aspects and embodiments of the present disclosure, interaction between certain CH3 domain-bearing polypeptides is attenuated/inhibited/reduced through the introduction of one or more destabilising modifications in a CH3 domain, for destabilising association between the CH3 domain and the CH3 domain of another CH3 domain-bearing polypeptide.

As used herein, a ‘destabilising modification’ refers to a modification which attenuates/inhibits/reduces association between the CH3 domain comprising the modification, and another CH3 domain. In some embodiments, a destabilising modification is a modification to a CH3 domain, that-when introduced in isolation, i.e. into an otherwise unmodified CH3 domain-attenuates/inhibits/reduces association between the CH3 domain comprising the modification and the equivalent unmodified CH3 domain lacking the modification.

In some embodiments, a destabilising modification is a modification to a CH3 domain, that when introduced into a first CH3 domain otherwise comprising the amino acid sequence of one of SEQ ID NOs: 1, 2, 3, 4, 5, 6, 7, 8, 9 or 10, attenuates/inhibits/reduces association between the first CH3 domain and a second CH3 domain comprising the equivalent amino acid sequence lacking the modification.

By way of illustration, introduction of 357K into a CH3 domain otherwise having the amino acid sequence of SEQ ID NO:1 attenuates/inhibits/reduces association between the modified CH3 domain and a CH3 domain having the amino acid sequence of SEQ ID NO:1. Similarly, introduction of 370E into a CH3 domain otherwise having the amino acid sequence of SEQ ID NO: 1 attenuates/inhibits/reduces association between the modified CH3 domain and a CH3 domain having the amino acid sequence of SEQ ID NO:1.

In some embodiments, a destabilising modification comprises, or consists of, modification to a position of a CH3 provided at, or proximal to, the point of interface when two CH3 domains interact with one another through protein-protein interactions.

In some embodiments, a destabilising modification changes the charge of the amino acid residue at the relevant position of the CH3 domain, relative to the equivalent CH3 domain lacking the modification. By way of illustration, the destabilising modification E357K replaces a negatively-charged glutamate residue with a positively-charged lysine residue. Similarly, the destabilising modification K370E replaces a positively-charged lysine residue with a negatively-charged glutamate residue.

In some embodiments, a destabilising modification substitutes a negatively-charged amino acid residue (e.g. E or D) for a positively-charged amino acid residue (e.g. R, K or H) or an uncharged amino acid residue. In some embodiments, a destabilising modification substitutes a negatively-charged amino acid residue for a positively-charged amino acid residue.

In some embodiments, a destabilising modification substitutes a positively-charged amino acid residue (e.g. R, K or H) for a negatively-charged amino acid residue (e.g. E or D) or an uncharged amino acid residue. In some embodiments, a destabilising modification substitutes a positively-charged amino acid residue for a negatively-charged amino acid residue.

In some embodiments, a destabilising modification substitutes an uncharged amino acid residue for a negatively-charged amino acid residue (e.g. E or D) or a positively-charged amino acid residue (e.g. R, K or H). In some embodiments, a destabilising modification substitutes an uncharged amino acid residue for a negatively-charged amino acid residue. In some embodiments, a destabilising modification substitutes an uncharged amino acid residue for a positively-charged amino acid residue.

In some embodiments, a destabilising modification disrupts one or more protein-protein interactions between amino acid residue(s) on a first CH3 domain and amino acid residue(s) on a second CH3 domain, which interact on association between the first CH3 domain and the second CH3 domain (e.g. homotypic association between an identical first CH3 domain and second CH3 domain). The protein-protein interaction may e.g. be an electrostatic interaction (e.g. a salt bridge).

By way of illustration, the introduction of the destabilising modification E357K into a CH3 domain otherwise having the amino acid sequence of SEQ ID NO:1 disrupts the salt bridge ordinarily formed between the 357E COO⁻ group and the 370K NH₃⁺ group in CH3 homodimers having the amino acid sequence of SEQ ID NO:1.

In some embodiments, a destabilising modification of a CH3 domain introduces a repulsive charge with respect to the charge of an amino acid residue on a second CH3 domain with which the equivalent unmodified amino acid residue at the relevant position interacts on association between the first CH3 domain and the second CH3 domain (e.g. homotypic association between an identical first CH3 domain and second CH3 domain).

In some embodiments, a destabilising modification of a CH3 domain introduces an amino acid residue having a charge which is the same charge as the charge of an amino acid residue on a second CH3 domain with which the equivalent unmodified amino acid residue at the relevant position interacts on association between the first CH3 domain and the second CH3 domain (e.g. homotypic association between an identical first CH3 domain and second CH3 domain).

In some embodiments, a destabilising modification of a CH3 domain introduces an amino acid residue having a positive charge, wherein an amino acid residue of a second CH3 domain (with which the equivalent unmodified amino acid residue at the relevant position interacts on association between the first CH3 domain and the second CH3 domain (e.g. homotypic association between an identical first CH3 domain and second CH3 domain)) has a positive charge. In some embodiments, a destabilising modification of a CH3 domain introduces an amino acid residue having a negative charge, wherein an amino acid residue of a second CH3 domain (with which the equivalent unmodified amino acid residue at the relevant position interacts on association between the first CH3 domain and the second CH3 domain (e.g. homotypic association between an identical first CH3 domain and second CH3 domain)) has a negative charge.

By way of illustration, introduction of the destabilising modification E357K into a CH3 domain otherwise having the amino acid sequence of SEQ ID NO: 1 introduces a repulsive charge between the 357K NH₃⁺ group and the 370K NH₃⁺ group on interaction between a first CH3 domain comprising E357K otherwise having the amino acid sequence of SEQ ID NO: 1, and a second CH3 domain having the amino acid sequence of SEQ ID NO:1. Similarly, introduction of the destabilising modification K370E into a CH3 domain otherwise having the amino acid sequence of SEQ ID NO: 1 introduces a repulsive charge between the 370E COO⁻ group and the 357E COO group on interaction between a first CH3 domain comprising K370E otherwise having the amino acid sequence of SEQ ID NO:1, and a second CH3 domain having the amino acid sequence of SEQ ID NO:1.

In some embodiments, a destabilising modification of a CH3 domain does not destabilise interaction between the CH3 domain and another CH3 domain comprising a destabilising modification. In some embodiments, a destabilising modification of a CH3 domain stabilises interaction between the CH3 domain and another CH3 domain comprising a destabilising modification.

In some aspects and embodiments, a modification for destabilising association between the CH3 domain comprising the destabilising modification (CH3 domain ‘a’) and the CH3 domain of another CH3 domain-bearing polypeptide (CH3 domain ‘b’) stabilises association between CH3 domain ‘a’ and the CH3 domain of another CH3 domain-bearing polypeptide (CH3 domain ‘c’) which is different to CH3 domain ‘b’. That is, in some embodiments, the modification of CH3 domain ‘a’ which destabilises association between CH3 domain ‘a’ and CH3 domain ‘b’ stabilises association between CH3 domain ‘a’ and CH3 domain ‘c’.

Similarly, in some aspects and embodiments, a modification for stabilising association between the CH3 domain comprising the modification (CH3 domain ‘a’) and the CH3 domain of another CH3 domain-bearing polypeptide (CH3 domain ‘b’) destabilises association between CH3 domain ‘a’ and the CH3 domain of another CH3 domain-bearing polypeptide (CH3 domain ‘c’) which is different to CH3 domain ‘b’. That is, in some embodiments, the modification of CH3 domain ‘a’ which stabilises association between CH3 domain ‘a’ and CH3 domain ‘b’ destabilises association between CH3 domain ‘a’ and CH3 domain ‘c’.

In some aspects and embodiments, interaction between certain CH3 domain-bearing polypeptides is potentiated/promoted/increased through the introduction of a substitution in the CH3 domain for stabilising association between the CH3 domain and the CH3 domain of another CH3 domain-bearing polypeptide.

In some embodiments, a destabilising modification potentiates/promotes/increases association between the CH3 domain comprising the modification and another CH3 domain comprising a destabilising modification.

In some embodiments, polypeptide complexes according to the present disclosure comprise polypeptides comprising CH3 domains having complementary destabilising modifications.

In some embodiments, amino acid residues introduced by destabilising modifications of CH3 domains of polypeptides in polypeptide complexes according to the present disclosure form protein-protein interactions on association between the CH3 domains. The protein-protein interactions may e.g. be electrostatic interactions (e.g. salt bridges).

By way of illustration, in association between a first CH3 domain comprising the destabilising modification E357K and a second CH3 domain comprising the destabilising modification K370E, a salt bridge is formed between the 357K NH₃⁺ group, and the 370E COO group, stabilising interaction between the first CH3 domain and the second CH3 domain. Similarly, in association between a first CH3 domain comprising the destabilising modification D356K and a second CH3 domain comprising the destabilising modification K439E, a salt bridge is formed between the 356K NH₃⁺ group, and the 439E COO″ group, stabilising interaction between the first CH3 domain and the second CH3 domain.

In some embodiments, a destabilising modification of a first CH3 domain introduces an amino acid residue having a charge which is the opposite charge to the charge of an amino acid residue introduced by a destabilising modification of a second CH3 domain with which the amino acid residue introduced by the destabilising modification of the first CH3 domain interacts on association between the first CH3 domain and the second CH3 domain.

In some embodiments, a destabilising modification of a first CH3 domain introduces an amino acid residue having a positive charge, wherein the charge of an amino acid residue introduced by a destabilising modification of a second CH3 domain (with which the amino acid residue introduced by the destabilising modification of the first CH3 domain interacts on association between the first CH3 domain and the second CH3 domain) has a negative charge. In some embodiments, a destabilising modification of a first CH3 domain introduces an amino acid residue having a negative charge, wherein the charge of an amino acid residue introduced by a destabilising modification of a second CH3 domain (with which the amino acid residue introduced by the destabilising modification of the first CH3 domain interacts on association between the first CH3 domain and the second CH3 domain) has a positive charge.

By way of illustration, the modification E357K in the CH3 domain of human IgG1 destabilises association between the CH3 domain comprising E357K and another CH3 domain comprising E357K. Similarly, the modification K370E in the CH3 domain of human IgG1 destabilises association between the CH3 domain comprising K370E and another CH3 domain comprising K370E. However, the modification E357K stabilises association with a CH3 domain comprising the modification K370E, and similarly the modification K370E stabilises association with a CH3 domain comprising the modification E357K, through formation of a salt bridge between the 357K NH₃⁺ group, and the 370E COO⁻ group.

Modifications to a CH3 domain for promoting association with another CH3 domain, and modifications for destabilising association between CH3 domains, can be identified using assays for analysing protein-protein interaction. In particular, assays for analysing the level of interaction between polypeptides comprising CH3 domains may be used to identify such modifications.

Suitable techniques to be employed for the identification of such modifications include e.g. resonance energy transfer techniques such as fluorescence resonance energy transfer (FRET) and Bioluminescence Resonance Energy Transfer (BRET), using appropriate labelled interaction partners, e.g. as described in Ciruela, Curr. Opin. Biotechnol. (2008) 19 (4): 338-43. Other suitable technologies include protein-fragment complementation systems, e.g. NanoLuc and NanoBiT, which are described e.g. in Thirukkumaran et al., Front. Chem. (2020) 7:938 and Dixon et al., ACS Chem. Biol. (2016) 11 (2): 400-408.

A candidate modification may be introduced into the amino acid sequence of the CH3 domain of a first polypeptide comprising a CH3 domain, and the level of interaction between first polypeptide and a second polypeptide comprising a CH3 domain may be determined. The level of interaction between the first polypeptide (comprising the modification) and the second polypeptide may be compared to the level of interaction determined between the second polypeptide and a third polypeptide, which is identical to the first polypeptide except that it lacks the candidate modification. A modification that results in a level of interaction between the first polypeptide and the second polypeptide that is greater than the level of interaction between the third polypeptide and the second polypeptide may be identified as a modification for promoting association between a CH3 domain and the CH3 domain of the second polypeptide. A modification that results in a level of interaction between the first polypeptide and the second polypeptide that is less than the level of interaction between the third polypeptide and the second polypeptide may be identified as a modification for destabilising association between a CH3 domain and the CH3 domain of the second polypeptide.

Modifications to a CH3 domain for promoting association with another CH3 domain, and modifications for destabilising association between CH3 domains, can also be identified by analysis using EGAD software, as described e.g. in WO 2009/089004 A1 and Pokala, N. and Handel, T. M., J. Mol. Biol. 347 (2005) 203-227, both of which are hereby incorporated by reference in their entirety.

EGAD software can be used to estimate CH3-CH3 domain binding free energy, and can be used to infer the effect of a given modification in a CH3 domain on CH3-CH3 domain binding free energy. Briefly, the binding free energy of a given mutant CH3 comprising a modification is defined as ΔΔGmut=μ(ΔGmut−ΔGwt) (mut=mutant, wt=wild-type), where, μ(=0.1, in general) is the scaling factor used to normalise the predicted changes in binding affinity to have a slope of 1 when comparing with the experimental energies. The free energy of dissociation (ΔG) is defined as the energy difference between the complex (ΔGbound) and free states (ΔGfree).

A destabilising modification according to the present disclosure may be a modification to a CH3 domain that (on introduction into a CH3 domain of an interacting pair of CH3 domains) increases ΔG calculated according to EGAD (Pokala, N. and Handel, T. M., J. Mol. Biol. 347 (2005) 203-227), relative to ΔG calculated for the interaction between equivalent unmodified CH3 domains. Conversely, a modification for promoting association between CH3 domains may be a modification that (on introduction into a CH3 domain of an interacting pair of CH3 domains) decreases ΔG calculated according to EGAD, relative to ΔG calculated for the interaction between equivalent unmodified CH3 domains.

Modifications to a CH3 domain for promoting association with another CH3 domain, and modifications for destabilising association between CH3 domains, can also be identified as described in WO 2020/216883 A1, which is incorporated by reference herein. See in particular the experimental examples of WO 2020/216883 A1.

It will be appreciated that modifications to CH3 domains of polypeptides according to the present disclosure are typically selected in accordance with the desired production of payload-bearing polypeptide complexes, by polypeptide exchange of constituent polypeptides of precursor polypeptide complexes.

The CH3 domain of a polypeptide of a given precursor polypeptide complex may comprise modification promoting association between the polypeptides of the precursor polypeptide complex. For example, the polypeptides of a given precursor polypeptide complex may comprise complementary ‘knob’ and ‘hole’ modifications (described hereinbelow). One or both of the polypeptides of the precursor polypeptide complex further comprises a destabilising modification, for reducing the affinity of association between the first and second polypeptides/reducing the stability of the precursor polypeptide complex/increasing the propensity of the precursor polypeptide complex to disassociate. However, the destabilising modification does not completely prevent interaction between the polypeptides (and thus formation of the precursor polypeptide complex). Rather, the destabilising modification inhibits/reduces/destabilises interaction between the polypeptides of the given precursor complex to the extent that the affinity of interaction between its constituent polypeptides is lower than the affinity of interaction between a constituent polypeptide of the given precursor complex, and a constituent polypeptide of a second, different precursor complex. In this way, when the first and second polypeptide complexes are incubated with one another, polypeptide exchange occurs, yielding new polypeptide complexes comprising one polypeptide each from the first and second precursor polypeptide complexes.

By way of illustration, a first precursor polypeptide complex may comprise: (i) a first polypeptide, comprising a CH3 domain comprising the knob modification 366W, and the destabilising modification 370E; and (ii) a second polypeptide comprising the hole modification 407V, 366S, 368A. The first and second polypeptides interact via their CH3 domains to form a polypeptide complex, but the glutamate residue at 370 of the CH3 domain of the first polypeptide destabilises the complex (i.e. relative to the stability of the complex that would be formed between the two polypeptides in the absence of the destabilising modification (i.e. when the residue at 370 of CH3 domain of the first polypeptide is the wildtype lysine residue of human IgG1)), as it introduces charge repulsion between position 370 of the CH3 domain of the first polypeptide, and the glutamate residue at position 357 of the CH3 domain of the second polypeptide.

In such circumstances, a second precursor polypeptide complex may comprise: (i) a first polypeptide, comprising a CH3 domain comprising the knob modification 366W; and (ii) a second polypeptide comprising the hole modification 407V, 366S, 368A, and the destabilising modification 357K. Once again, the first and second polypeptides interact via their CH3 domains to form a polypeptide complex, but the lysine residue at 357 of the CH3 domain of the second polypeptide destabilises the complex (i.e. relative to the stability of the complex that would be formed between the two polypeptides in the absence of the destabilising modification (i.e. when the residue at 357 of CH3 domain of the second polypeptide is the wildtype glutamate residue of human IgG1)), as it introduces charge repulsion between position 357 of the CH3 domain of the second polypeptide, and the lysine residue at position 370 of the CH3 domain of the first polypeptide.

In this scenario, polypeptide exchange between the two precursor polypeptide complexes is favoured, in particular because the 370E residue of the CH3 domain of the first polypeptide of the first precursor polypeptide complex forms a salt bridge with the 357K residue of the CH3 domain of the second polypeptide of the second precursor polypeptide complex.

Thus two new, polypeptide-exchanged complexes are formed from the precursor complexes: (a) a polypeptide complex comprising the first polypeptide of the first precursor polypeptide complex and the second polypeptide of the second precursor polypeptide complex, and (b) a polypeptide complex comprising the second polypeptide of the first precursor polypeptide complex and the first polypeptide of the second precursor polypeptide complex.

It will be appreciated that the polypeptide complex (a) of the preceding paragraph is more stable/has a lower propensity to dissociate/is formed by polypeptides interacting with greater affinity, as compared to either of the precursor polypeptide complexes. Similarly, polypeptide complex (b) of the preceding paragraph is more stable/has a lower propensity to dissociate/is formed by polypeptides interacting with greater affinity, as compared to either of the precursor polypeptide complexes, because neither of its constituent polypeptides comprise a modification destabilising their association.

Modifications to CH3 domains for destabilising/stabilising association between CH3 domains bearing such modifications and certain other CH3 domains are described e.g. in WO 2019/077092 A1, Dengl et al., Nat. Commun. (2020) 11:4974 and WO 2020/216883 A1, all of which are hereby incorporated by reference in their entirety.

In some embodiments, a destabilising modification comprises, or consists of, modification at one or more of the following positions: 345, 347, 349, 351, 354, 356, 357, 360, 362, 364, 366, 368, 370, 390, 392, 394, 397, 399, 400, 401, 405, 407, 409, 439 or 441. Accordingly, in some embodiments a CH3 domain comprising a destabilising modification comprises modification at one or more of the following positions: 345, 347, 349, 351, 354, 356, 357, 360, 362, 364, 366, 368, 370, 390, 392, 394, 397, 399, 400, 401, 405, 407, 409, 439 or 441.

In some embodiments, a destabilising modification comprises, or consists of: replacement of Q347 with a positively-charged amino acid, and replacement of K360 with a negatively-charged amino acid; replacement of Y349 with a negatively-charged amino acid; replacement of L351 with a hydrophobic amino acid, and replacement of E357 with a hydrophobic amino acid; replacement of S364 with a hydrophobic amino acid; replacement of W366 with a hydrophobic amino acid, and replacement of K409 with a negatively-charged amino acid; replacement of L368 with a hydrophobic amino acid; replacement of K370 with a negatively-charged amino acid; replacement of K370 with a negatively-charged amino acid, and replacement of K439 with a negatively-charged amino acid; replacement of K392 with a negatively-charged amino acid; replacement of T394 with a hydrophobic amino acid; replacement of V397 with a hydrophobic amino acid; replacement of D399 with a positively-charged amino acid, and replacement of K409 with a negatively-charged amino acid; replacement of $400 with a positively-charged amino acid; F405W; Y407W; replacement of K439 with a negatively-charged amino acid; replacement of S354 with a hydrophobic amino acid; replacement of D356 with a positively-charged amino acid; replacement of E357 with a positively-charged amino acid or a hydrophobic amino acid; replacement of D356 with a positively-charged amino acid, and replacement of E357 with a positively-charged amino acid or a hydrophobic amino acid; replacement of S364 with a hydrophobic amino acid; replacement of A368 with a hydrophobic amino acid; replacement of K392 with a negatively-charged amino acid; replacement of T394 with a hydrophobic amino acid; replacement of D399 with a hydrophobic amino acid, and replacement of S400 with a positively-charged amino acid; replacement of D399 with a hydrophobic amino acid, and replacement of F405 with a positively-charged amino acid; replacement of V407 with a hydrophobic amino acid; replacement of K409 with a negatively-charged amino acid; and replacement of K439 with a negatively-charged amino acid.

In some embodiments, a CH3 domain comprising a knob modification comprises a destabilising modification comprising, or consisting of: replacement of Q347 with a positively-charged amino acid, and replacement of K360 with a negatively-charged amino acid; replacement of Y349 with a negatively-charged amino acid; replacement of L351 with a hydrophobic amino acid, and replacement of E357 with a hydrophobic amino acid; replacement of S364 with a hydrophobic amino acid; replacement of W366 with a hydrophobic amino acid, and replacement of K409 with a negatively-charged amino acid; replacement of L368 with a hydrophobic amino acid; replacement of K370 with a negatively-charged amino acid; replacement of K370 with a negatively-charged amino acid, and replacement of K439 with a negatively-charged amino acid; replacement of K392 with a negatively-charged amino acid; replacement of T394 with a hydrophobic amino acid; replacement of V397 with a hydrophobic amino acid; replacement of D399 with a positively-charged amino acid, and replacement of K409 with a negatively-charged amino acid; replacement of S400 with a positively-charged amino acid; F405W; Y407W; and replacement of K439 with a negatively-charged amino acid.

In some embodiments, a CH3 domain comprising a hole modification comprises a destabilising modification comprising, or consisting of: replacement of S354 with a hydrophobic amino acid; replacement of D356 with a positively-charged amino acid; replacement of E357 with a positively-charged amino acid or a hydrophobic amino acid; replacement of D356 with a positively-charged amino acid, and replacement of E357 with a positively-charged amino acid or a hydrophobic amino acid; replacement of S364 with a hydrophobic amino acid; replacement of A368 with a hydrophobic amino acid; replacement of K392 with a negatively-charged amino acid; replacement of T394 with a hydrophobic amino acid; replacement of D399 with a hydrophobic amino acid, and replacement of S400 with a positively-charged amino acid; replacement of D399 with a hydrophobic amino acid, and replacement of F405 with a positively-charged amino acid; replacement of V407 with a hydrophobic amino acid; replacement of K409 with a negatively-charged amino acid; and replacement of K439 with a negatively-charged amino acid.

In some embodiments, a CH3 domain comprising a destabilising modification comprises one or more of the following: 345R; 347K; 349W or 349E; 351F or 351Y; 354E or 354V; 356S, 356A or 356K; 357S, 357A, 357L, 357F or 357K; 360S or 360E; 362E; 364V or 364L; 366I; 368F or 368V; 370E; 390E; 392E or 392D; 394I; 397Y; 399A or 399K; 400K; 401R; 405W; 407W, 407L or 407I; 409D, 409E or 409I; 439E; or 441Y. In some embodiments, a CH3 domain comprising a destabilising modification comprises 357S, 357A, 357L, 357F or 357K (e.g. 357K). In some embodiments, a CH3 domain comprising a destabilising modification comprises 370E. In some embodiments, a CH3 domain comprising a destabilising modification comprises 356S, 356A or 356K (e.g. 356K). In some embodiments, a CH3 domain comprising a destabilising modification comprises 439E.

In some embodiments, a destabilising modification is or comprises: E345R; Q347K; Y349W or Y349E; L351F or L351Y; S354E or S354V; D356S, D356A or D356K; E357S, E357A, E357L, E357F or E357K; K360S or K360E; Q362E; S364V or S364L; T366I; L368F or L368V; K370E; N390E; K392E or K392D; T394I; V397Y; D399A or D399K; S400K; D401R; F405W; Y407W, Y407L or Y407I; K409D, K409E or K409I; K439E; or L441Y. In some embodiments, a destabilising modification is or comprises E357S, E357A, E357L, E357F or E357K (e.g. E357K). In some embodiments, a destabilising modification is or comprises K370E. In some embodiments, a destabilising modification is or comprises D356S, D356A or D356K (e.g. D356K). In some embodiments, a destabilising modification is or comprises K439E.

In some embodiments, a polypeptide complex according to the present disclosure comprises: (i) a first polypeptide having a CH3 domain comprising 357S, 357A, 357L, 357F or 357K (e.g. 357K), and (ii) a second polypeptide having a CH3 domain comprising 370E.

In some embodiments, a polypeptide complex according to the present disclosure comprises: (i) a first polypeptide having a CH3 domain comprising 356S, 356A or 356K (e.g. 356K), and (ii) a second polypeptide having a CH3 domain comprising 439E.

In some embodiments, a polypeptide complex according to the present disclosure comprises: (i) a first polypeptide having a CH3 domain comprising the destabilising modification E357S, E357A, E357L, E357F or E357K (e.g. E357K), and (ii) a second polypeptide having a CH3 domain comprising the destabilising modification K370E.

In some embodiments, a polypeptide complex according to the present disclosure comprises: (i) a first polypeptide having a CH3 domain comprising the destabilising modification D356S, D356A or D356K (e.g. D356K), and (ii) a second polypeptide having a CH3 domain comprising the destabilising modification K439E.

In some embodiments, a destabilising modification attenuates/inhibits/reduces association between CH3 domains comprising modification to promote their association. Such destabilising modifications may be ‘back mutations’, reverting to the amino acid residue of the CH3 domain in the absence of the modifications for promoting association between CH3 domains.

In some embodiments, a destabilising modification attenuates/inhibits/reduces association between a CH3 domain comprising a knob modification (e.g. a knob-cys modification), and a CH3 domain comprising a hole modification (e.g. a hole-cys modification). Such destabilising modifications may be ‘back mutations’, reverting to the amino acid residue of the CH3 domain in the absence of the knob/hole modification.

In some embodiments, the destabilising modification disrupts/partially disrupts the knob structure in a CH3 domain comprising a knob modification. In some embodiments, the destabilising modification disrupts/partially disrupts the hole structure in a CH3 domain comprising a hole modification. In some embodiments, a destabilising modification to a CH3 domain comprising a knob or hole modification may partly or completely remove the knob/hole modification. In some embodiments, a CH3 domain comprising a knob or hole modification and further comprising a destabilising modification may have the amino acid sequence of a CH3 domain lacking the knob/hole modification.

In some embodiments, a CH3 domain comprising a knob modification and further comprising a destabilising modification does not comprise W and/or does not comprise Y at position 366. In some embodiments, a CH3 domain comprising a knob modification and further comprising a destabilising modification comprises 366T.

In some embodiments, a CH3 domain comprising a hole modification and further comprising a destabilising modification does not comprise V and/or does not comprise A and/or does not comprise S and/or does not comprise T at position 407. In some embodiments, a CH3 domain comprising a hole modification and further comprising a destabilising modification comprises 407Y.

In some embodiments, a CH3 domain comprising a hole modification and further comprising a destabilising modification does not comprise S and/or does not comprise V and/or does not comprise A at position 366. In some embodiments, a CH3 domain comprising a hole modification and further comprising a destabilising modification comprises 366T.

In some embodiments, a CH3 domain comprising a hole modification and further comprising a destabilising modification does not comprise A and/or does not comprise V and/or does not comprise S and/or does not comprise T at position 368. In some embodiments, a CH3 domain comprising a hole modification and further comprising a destabilising modification comprises 368L.

In some embodiments, a CH3 domain comprising a knob-cys modification and further comprising a destabilising modification does not comprise C at position 354. In some embodiments, a CH3 domain comprising a knob-cys modification and further comprising a destabilising modification comprises 354S.

In some embodiments, a CH3 domain comprising a hole-cys modification and further comprising a destabilising modification does not comprise C at position 349. In some embodiments, a CH3 domain comprising a hole-cys modification and further comprising a destabilising modification comprises 349Y.

In some embodiments, a polypeptide complex according to the present disclosure comprises: (i) a polypeptide having a CH3 domain comprising a knob modification, and at least one modification selected from: Y349E; Y349D; S364V; S364I; S364L; L368F; K370E; K370D; K392E; K392D; T394L; T394I; V397Y; S400K; S400R; F405W; Y407W; K349E; K439D; Q347K and K360E; Q347R; K360E; Q347K and K360D; Q347R and K360D; L351F and E357F; W366I and K409E; W366L and K409E; W366K and K409D; W366L and K409D; D399K and K409E; D399R and K409E; D399K and K409D; and D399K and K409E; and (ii) a polypeptide having a CH3 domain comprising a hole modification, and at least one modification selected from: S354V; S354I; S354L; D356K; D356R; E357K; E357R; E357F; S364L; S364I; A368F; K392D; K392E; T394L; T394I; V407Y; K409E; K409D; K439D; K439E; D399A and S400K; D399A and S400R; D399A and F405W.

In some embodiments, a polypeptide complex according to the present disclosure comprises: (i) a polypeptide having a CH3 domain comprising a knob modification, and at least one modification selected from: Y349E; S364V; L368F; K370E; K392D; T394I; V397Y; S400K; F405W; Y407W; K349E; Q347K and K360E; L351F and E357F; W366I and K409E; and D399K and K409E; and (ii) a polypeptide having a CH3 domain comprising a hole modification, and at least one modification selected from: S354V; D356K; E357K; E357F; S364L; A368F; K392E; T394I; V407Y; K409E; K439E; and D399A and S400K.

In some embodiments, a polypeptide complex according to the present disclosure comprises: (i) a polypeptide having a CH3 domain comprising a knob modification, and at least one modification selected from: Y349E; S364V; L368F; K370E; K392D; T394I; V397Y; S400K; F405W; Y407W; K349E; Q347K and K360E; L351F and E357F; W366I and K409E; and D399K and K409E; and (ii) a polypeptide having a CH3 domain comprising a hole modification, and at least one modification selected from: D356K; D356R; E357K; E357R; E357F; S364L; S364I; V407Y; K409E; K409D; D399A and S400K; and D399A and S400R.

In some embodiments, a polypeptide complex according to the present disclosure comprises: (i) a polypeptide having a CH3 domain comprising a knob modification, and at least one modification selected from: Y349E; K370E; K392D; T394I; V397Y; F405W; Y407W; K349E; Q347K and K360E; W366I and K409E; and D399K and K409E; and (ii) a polypeptide having a CH3 domain comprising a hole modification, and at least one modification selected from: D356K; E357K; E357F; S364L; V407Y; K409E; and D399A and S400K.

In some embodiments, a polypeptide complex according to the present disclosure comprises: (i) a polypeptide having a CH3 domain comprising a knob modification (e.g. T366W or T366W and S354C), and the modification(s) of one of rows 1 to 49 of column A of Table II; and (ii) a polypeptide having a CH3 domain comprising a hole modification (e.g. Y407V, T366S and L368A; or Y407V, T366S, L368A and Y349C), and the modification(s) of one of rows 1 to 49 of column B of Table II:

TABLE II

| | CH3 domain comprising | CH3 domain comprising |
|---|---|---|
| | knob modification | hole modification |
| | (Column A) | (Column B) |
| 1 | K370E and K439E | D356K |
| 2 | Y349E | D356K and E357K |
| 3 | W366I and K409E | D356K and E357K |
| 4 | K370E and K439E | D356K and E357K |
| 5 | K392D | D356K and E357K |
| 6 | T394I | D356K and E357K |
| 7 | D399K and K409E | D356K and E357K |
| 8 | F405W | D356K and E357K |
| 9 | Y407W | D356K and E357K |
| 10 | Y349E | E357K |
| 11 | W366I and K409E | E357K |
| 12 | K370E and K439E | E357K |
| 13 | T394I | E357K |
| 14 | D399K and K409E | E357K |
| 15 | F405W | E357K |
| 16 | Y407W | E357K |
| 17 | Q347K and K360E | E357F |
| 18 | Y349E | E357F |
| 19 | W366I and K409E | E357F |
| 20 | K370E and K439E | E357F |
| 21 | T394I | E357F |
| 22 | D399K and K409E | E357F |
| 23 | Y407W | E357F |
| 24 | Y349E | S364L |
| 25 | K392D | S364L |
| 26 | F405W | S364L |
| 27 | W366I and K409E | S364L |
| 28 | K370E and K439E | S364L |
| 29 | T394I | S364L |
| 30 | D399K and K409E | S364L |
| 31 | Y407W | S364L |
| 32 | T394I | A368F |
| 33 | W366I and K409E | D399A and S400K |
| 34 | T394I | D399A and S400K |
| 35 | Q347K and K360E | V407Y |
| 36 | Y349E | V407Y |
| 37 | W366I and K409E | V407Y |
| 38 | T394I | V407Y |
| 39 | D399K and K409E | V407Y |
| 40 | F405W | V407Y |
| 41 | Y407W | V407Y |
| 42 | K392D | V407Y |
| 43 | Y349E | K409E |
| 44 | W366I and K409E | K409E |
| 45 | K392D | K409E |
| 46 | T394I | K409E |
| 47 | D399K and K409E | K409E |
| 48 | F405W | K409E |
| 49 | Y407W | K409E |

In preferred embodiments, for a given polypeptide complex, the modification(s) of the CH3 domain comprising a knob modification from column A and the modification(s) of the CH3 domain comprising a hole modification from column B are selected from the same row of Table II. For example, in some embodiments, a polypeptide complex according to the present disclosure comprises: (i) a polypeptide having a CH3 domain comprising a knob modification (e.g. T366W or T366W and S354C), and the modifications K370E and K439E (row 1 of column A); and (ii) a polypeptide having a CH3 domain comprising a comprising a hole modification (e.g. Y407V, T366S and L368A; or Y407V, T366S, L368A and Y349C), and the modification D356K (row 1 of column B).

In some embodiments, a polypeptide complex according to the present disclosure comprises: (i) a polypeptide having a CH3 domain comprising a knob modification (e.g. T366W or T366W and S354C), and the modification(s) of one of rows 1 to 12 of column A of Table III; and (ii) a polypeptide having a CH3 domain comprising a hole modification (e.g. Y407V, T366S and L368A; or Y407V, T366S, L368A and Y349C), and the modification(s) of one of rows 1 to 12 of column B of Table III (in preferred embodiments, for a given polypeptide complex, the modification(s) of the CH3 domain comprising a knob modification from column A and the modification(s) of the CH3 domain comprising a hole modification from column B are selected from the same row of Table III):

TABLE III

| | CH3 domain comprising | CH3 domain comprising |
|---|---|---|
| | knob modification | hole modification |
| | (Column A) | (Column B) |
| 1 | K370E | V407Y |
| 2 | K370E K439E | V407Y |
| 3 | V397Y | V407Y |
| 4 | K439E | S364L |
| 5 | W366I and K409E | S364L |
| 6 | K370E and K439E | S364L |
| 7 | D399K and K409E | S364L |
| 8 | T394I | S364L |
| 9 | W366I and K409E | S364L |
| 10 | K370E and K439E | S364L |
| 11 | D399K and K409E | S364L |
| 12 | T394I | S364L |

In some embodiments, the CH3 domain comprising a knob modification either does not comprise a destabilising modification, or comprises at least one modification selected from: K370E; K370D; K392E; K392D; V397Y; K370E and K439E; K370D and K439E; K370E and K439D; and K370D and K439D; and the CH3 domain comprising a hole modification comprises at least one modification selected from: E357K; E357R; S364L; S364I; V407Y; V407F and A368F.

In some embodiments, the CH3 domain comprising a knob modification comprises at least one modification selected from: K370E; K392D; V397Y; K370E and K439E; and the CH3 domain comprising a hole modification comprises at least one modification selected from: E357K; S364L; V407Y; and A368F.

In some embodiments, the CH3 domain comprising a knob modification comprises at least one modification selected from: K370E; K370D; K392E; K392D; V397Y; K370E and K439E; K370D and K439E; K370E and K439D; and K370D and K439D; and the CH3 domain comprising a hole modification comprises at least one modification selected from: E357K; E357R; S364L; S364I; V407Y; and V407F.

In some embodiments, the CH3 domain comprising a knob modification comprises at least one modification selected from: K370E; K392D; V397Y; and K370E and K439E; and the CH3 domain comprising a hole modification comprises at least one modification selected from: E357K; S364L; and V407Y.

In some embodiments, a polypeptide complex according to the present disclosure comprises: (i) a polypeptide having a CH3 domain comprising a knob modification (e.g. T366W or T366W and S354C), and the modification(s) of one of rows 1 to 14 of column A of Table IV; and (ii) a polypeptide having a CH3 domain comprising a hole modification (e.g. Y407V, T366S and L368A; or Y407V, T366S, L368A and Y349C), and the modification(s) of one of rows 1 to 14 of column B of Table IV (in preferred embodiments, for a given polypeptide complex, the modification(s) of the CH3 domain comprising a knob modification from column A and the modification(s) of the CH3 domain comprising a hole modification from column B are selected from the same row of Table IV):

TABLE IV

| | CH3 domain comprising | CH3 domain comprising |
|---|---|---|
| | knob modification | hole modification |
| | (Column A) | (Column B) |
| 1 | V397Y | E357K |
| 2 | K370E | E357K |
| 3 | K392D | E357K |
| 4 | K370E and K439E | E357K |
| 5 | no destabilising modification | V407Y |
| 6 | V397Y | V407Y |
| 7 | K370E | V407Y |
| 8 | no destabilising modification | S364L |
| 9 | V397Y | S364L |
| 10 | K370E | S364L |
| 11 | V397Y | A368F |
| 12 | K370E | A368F |
| 13 | K392D | A368F |
| 14 | K370E and K439E | A368F |

In some embodiments, a polypeptide complex according to the present disclosure comprises: (i) a polypeptide having a CH3 domain comprising a knob modification (e.g. T366W or T366W and S354C), and the modification(s) of one of rows 1 to 8 of column A of Table V; and (ii) a polypeptide having a CH3 domain comprising a hole modification (e.g. Y407V, T366S and L368A; or Y407V, T366S, L368A and Y349C), and the modification(s) of one of rows 1 to 8 of column B of Table V (in preferred embodiments, for a given polypeptide complex, the modification(s) of the CH3 domain comprising a knob modification from column A and the modification(s) of the CH3 domain comprising a hole modification from column B are selected from the same row of Table V):

TABLE V

| | CH3 domain comprising knob | CH3 domain comprising hole |
|---|---|---|
| | modification (Column A) | modification (Column B) |
| 1 | V397Y | E357K |
| 2 | K370E | E357K |
| 3 | K392D | E357K |
| 4 | K370E and K439E | E357K |
| 5 | V397Y | V407Y |
| 6 | K370E | V407Y |
| 7 | V397Y | S364L |
| 8 | K370E | S364L |

In some embodiments, a polypeptide complex according to the present disclosure (in particular, a precursor polypeptide complex according to the present disclosure) comprises: (i) a polypeptide having a CH3 domain comprising a knob modification (e.g. T366W or T366W and S354C), and the modification(s) of one of rows 1 to 18 of column A of Table VI; and (ii) a polypeptide having a CH3 domain comprising a hole modification (e.g. Y407V, T366S and L368A; or Y407V, T366S, L368A and Y349C), and the modification(s) of one of rows 1 to 18 of column B of Table VI (in preferred embodiments, for a given polypeptide complex, the modification(s) of the CH3 domain comprising a knob modification from column A and the modification(s) of the CH3 domain comprising a hole modification from column B are selected from the same row of Table VI):

TABLE VI

| | CH3 domain comprising knob | CH3 domain comprising hole |
|---|---|---|
| | modification (Column A) | modification (Column B) |
| 1 | K370E and K439E | E357F and S364L |
| 2 | Y349E, W366I and K409D | E357F and S364L |
| 3 | Y349E and K439E | E357F and S364L |
| 4 | K370E and K439E | D356K and V407Y |
| 5 | Y407W | E357F and S364L |
| 6 | Y349E, W366I and K409D | D356K and V407Y |
| 7 | Y349E and Y407W | E357F and S364L |
| 8 | Y349E and K439E | D356K and V407Y |
| 9 | W366I, F405W and K409D | E357F and S364L |
| 10 | D399K and K409E | E357F and S364L |
| 11 | W366I, K409D and K439E | D356K and V407Y |
| 12 | Y349E and K439E | S364I |
| 13 | D399K and K409E | D356K and V407Y |
| 14 | W366I and K409D | D356K and V407Y |
| 15 | T394I and F405W | D356K and V407Y |
| 16 | D399K and K409E | V407Y |
| 17 | D399K and K409E | E357K and T394I |
| 18 | W366I and K409D | E357K and T394I |

Precursor polypeptide complexes comprising polypeptides having CH3 domains comprising such combinations of destabilising modifications exhibit particularly beneficial polypeptide chain exchange.

In some embodiments, a polypeptide complex according to the present disclosure (in particular, a precursor polypeptide complex according to the present disclosure) comprises: (i) a polypeptide having a CH3 domain comprising a knob modification (e.g. T366W or T366W and S354C), and the modification(s) of one of rows 1 to 24 of column A of Table VII; and (ii) a polypeptide having a CH3 domain comprising a hole modification (e.g. Y407V, T366S and L368A; or Y407V, T366S, L368A and Y349C), and the modification(s) of one of rows 1 to 24 of column B of Table VII (in preferred embodiments, for a given polypeptide complex, the modification(s) of the CH3 domain comprising a knob modification from column A and the modification(s) of the CH3 domain comprising a hole modification from column B are selected from the same row of Table VII):

TABLE VII

| | CH3 domain comprising knob | CH3 domain comprising hole |
|---|---|---|
| | modification (Column A) | modification (Column B) |
| 1 | Y349D | E357F and S364L |
| 2 | Y349D | S364A |
| 3 | Y407W | E357F and S364L |
| 4 | Y349E | E357F and S364L |
| 5 | Y349E | S364A |
| 6 | W366I, F405W and K409D | E357F and S364L |
| 7 | W366I, F405W and K409D | S364A |
| 8 | D399K and K409E | E357F and S364L |
| 9 | D399K and K409E | S364A |
| 10 | Y349D | S364I |
| 11 | Y407W | S364I |
| 12 | Y349E | S364I |
| 13 | W366I, F405W and K409D | S364I |
| 14 | Y349D | S364L |
| 15 | D399K and K409E | S364I |
| 16 | Y407W | S364L |
| 17 | Y349D | K409E |
| 18 | Y349E | S364L |
| 19 | W366I, F405W and K409D | S364L |
| 20 | Y407W | K409E |
| 21 | Y349E | K409E |
| 22 | D399K and K409E | S364L |
| 23 | W366I, F405W and K409D | K409E |
| 24 | D399K and K409E | K409E |

Precursor polypeptide complexes comprising polypeptides having CH3 domains comprising such combinations of destabilising modifications exhibit particularly beneficial polypeptide chain exchange.

### Further Possible Modifications to CH3 Domains

In some aspects and embodiments, a CH3 domain of the present disclosure comprises modification to modify an Fc-mediated function.

Modifications to antibody Fc regions that influence Fc-mediated functions are known in the art, such as those described e.g. in Wang et al., Protein Cell (2018) 9 (1): 63-73, which is hereby incorporated by reference in its entirety. Exemplary Fc region modifications known to influence antibody effector function are summarised in Table 1 of Wang et al., Protein Cell (2018) 9 (1): 63-73.

In some embodiments, a CH3 domain of the present disclosure comprises a modification that would, when provided in an Fc region comprising the CH3 domain, increase or decrease the level of an Fc-mediated function (i.e. as compared to the level of the Fc-mediated function displayed by the equivalent Fc region lacking the modification).

In some embodiments, a CH3 domain comprises modification to increase an Fc-mediated function. In some embodiments, a CH3 domain comprises modification to increase ADCC, ADCP and/or CDC. In some embodiments, a CH3 domain comprises modification to increase binding to an Fc receptor (e.g. an Fcγ receptor, e.g. one or more of FcγRI, FcγRIIa, FcγRIIb, FcγRIIc, FcγRIIIa and FcγRIIIb). In some embodiments, a CH3 domain comprises modification to increase binding to FcRn. In some embodiments, a CH3 domain comprises modification to increase binding to a complement protein (e.g. Clq).

In some embodiments, a CH3 domain comprises modification to decrease an Fc-mediated function. In some embodiments, a CH3 domain comprises modification to decrease ADCC, ADCP and/or CDC. In some embodiments, a CH3 domain comprises modification to decrease binding to an Fc receptor (e.g. an Fcγ receptor, e.g. one or more of FcγRI, FcγRIIa, FcγRIIb, FcγRIIc, FcγRIIIa and FcγRIIIb). In some embodiments, a CH3 domain comprises modification to decrease binding to FcRn. In some embodiments, a CH3 domain comprises modification to decrease binding to a complement protein (e.g. Clq).

In embodiments and aspects of the present disclosure, a CH3 domain—e.g. a CH3 domains of a constituent polypeptide of a polypeptide complex according to the present disclosure-comprises a CH3 domain modification known to influence Fc-mediated function described in Wang et al., Protein Cell (2018) 9 (1): 63-73.

### Payload Moieties

Aspects and embodiments of the present disclosure relate to polypeptide complexes comprising payload moieties, and polypeptides comprising payload moieties.

Herein, a ‘payload moiety’ refers any moiety to be provided to a polypeptide complex. In some embodiments a payload moiety, is or comprises, a detectable moiety (e.g. a fluorescent moiety, luminescent moiety, radiopaque/contrast agent, radiolabel, immuno-detectable moieties), a moiety having a detectable activity (e.g. an enzymatic moiety) or a drug moiety (e.g. a cytotoxic moiety).

In some embodiments, a payload moiety is, or comprises, a detectable moiety. A ‘detectable moiety’ refers to moiety capable of producing a detectable signal indicative of its presence. Detectable moieties include moieties detectable by spectroscopic, photochemical, biochemical, immunochemical, electrical, optical or chemical means.

In some embodiments, a payload moiety is, or comprises, a fluorescent moiety. Fluorescent moieties are well known in the art, and include e.g. fluorescein, rhodamine, tetramethyl rhodamine, allophycocyanin, phycoerytherin, phycocyanin, cyanines (e.g. Cy2, Cy3, Cy3B, Cy3.5, C5, Cy5.5, Cy7), 4-methyl umbelliferone, 7-amino-4-methyl coumarin, o-phthaldehyde, fluorescamine, eosine, nitrobenzoxadiazole, Texas Red, green fluorescent protein (GFP), and chelates of rare earth metals such as europium (Eu), terbium (Tb) and samarium (Sm).

In some embodiments, a payload moiety is, or comprises, a luminescent moiety. Luminescent moieties include e.g. radioluminescent (e.g. radium, promethium, tritium), chemiluminescent (e.g. an acridinium ester, an imidazole, an acridinium salt, an oxalate ester acridinium ester, luminol, isoluminol) and bioluminescent moieties (e.g. a luciferin (e.g. coelenterazine), aequorin).

In some embodiments, a payload moiety is, or comprises, a radiopaque or contrast agent. Such agents include e.g. barium, diatrizoate, ethiodized oil, gallium citrate, iocarmic acid, iocetamic acid, iodamide, iodipamide, iodoxamic acid, iogulamide, iohexol, iopamidol, iopanoic acid, ioprocemic acid, iosefamic acid, ioseric acid, iosulamide meglumine, iosemetic acid, iotasul, iotetric acid, iothalamic acid, iotroxic acid, ioxaglic acid, ioxotrizoic acid, ipodate, meglumine, metrizamide, metrizoate, propyliodone, and thallous chloride.

In some embodiments, a payload moiety is, or comprises, a radiolabel. Radiolabels include radioisotopes such as Hydrogen³, Sulfur³⁵, Carbon¹⁴¹⁴, Phosphorus³², Iodine¹²³, Iodine¹²⁵, Iodine¹²⁶, Iodine¹³¹, Iodine¹³³, Bromine⁷⁷, Technetium⁹⁹m, Indium¹¹¹, Indium¹¹³m, Gallium⁶⁷, Gallium⁶⁸, Ruthenium⁹⁵, Ruthenium⁹⁷, Ruthenium¹⁰³, Ruthenium¹⁰⁵, Mercury²⁰⁷, Mercury²⁰³, Rhenium⁹⁹m, Rhenium¹⁰¹, Rhenium¹⁰⁵, Scandium⁴⁷, Tellurium¹²¹m, Tellurium¹²²m, Tellurium ¹²⁵m, Thulium¹⁶⁵, Thulium¹¹⁶⁷, Thulium¹⁶⁸, Copper⁶⁷, Fluorine¹⁸, Yttrium⁹⁰, Palladium¹⁰⁰, Bismuth²¹⁷ and Antimony²¹¹.

In some embodiments, a payload moiety is, or comprises, an immuno-detectable moiety. An immuno-detectable moiety is a moiety detectable using immunological techniques, e.g. using an antibody. Immuno-detectable moieties include e.g. peptide/polypeptide epitope tags, antibodies, receptors, ligands and nucleic acids. Immuno-detectable moieties include e.g. epitope tags, such as e.g. 6xHis, FLAG, c-Myc, StrepTag, haemagglutinin, calmodulin-binding protein (CBP), glutathione-s-transferase (GST), maltose-binding protein (MBP), thioredoxin, S-peptide, T7 peptide, SH2 domain, avidin, streptavidin, and haptens (e.g. biotin, digoxigenin, dinitrophenol).

In some embodiments, a payload moiety is, or comprises, a moiety having a detectable activity, e.g. an enzymatic moiety. Enzymatic moieties include e.g. luciferases, glucose oxidases, galactosidases (e.g. beta-galactosidase), glucorinidases, phosphatases (e.g. alkaline phosphatase), peroxidases (e.g., horseradish peroxidase) and cholinesterases.

In some embodiments, a payload moiety is, or comprises, a drug moiety. A drug moiety may be a chemical moiety for providing a therapeutic effect. A drug moiety may be a small molecule (e.g. a low molecular weight (<1000 daltons, typically between ˜300-700 daltons) organic compound). Drug moieties are described e.g. in Parslow et al., Biomedicines. (2016) Sep; 4 (3): 14 (hereby incorporated by reference in its entirety). In some embodiments, a drug moiety may be or comprise a cytotoxic agent. In some embodiments, a drug moiety may be or comprise a chemotherapeutic agent. Drug moieties include e.g. calicheamicin, DM1, DM4, monomethylauristatin E (MMAE), monomethylauristatin F (MMAF), SN-38, doxorubicin, duocarmycin, D6.5 and PBD.

In some embodiments, a payload moiety does not comprise or consist of a VH and/or a VL domain of an antibody. In some embodiments, a payload moiety does not comprise or consist of an antibody or an antibody fragment/derivative. In some embodiments, a payload moiety does not comprise or consist of a target-binding moiety.

Payload moieties of the present disclosure may be attached/conjugated to a polypeptide of the disclosure (e.g. a polypeptide of a polypeptide complex of the present disclosure) by any suitable means, which are well known in the art.

In some embodiments, a payload moiety may be covalently linked (e.g. chemically conjugated) a polypeptide of the present disclosure. Chemical conjugation may be performed by any suitable means and the skilled person will be well aware of suitable technologies, including, but not limited to: (1) direct coupling via protein functional groups (e.g., thiol-thiol linkage, amine-carboxyl linkage, amine-aldehyde linkage; enzyme direct coupling); (2) homobifunctional coupling of amines (e.g., using bis-aldehydes); (3) homobifunctional coupling of thiols (e.g., using bis-maleimides); (4) homobifunctional coupling via photoactivated reagents; (5) heterobifunctional coupling of amines to thiols (e.g., using maleimides); (6) heterobifunctional coupling via photoactivated reagents (e.g., the β-carbonyldiazo family); (7) introducing amine-reactive groups into a poly- or oligosaccharide via cyanogen bromide activation or carboxymethylation; (8) introducing thiol-reactive groups into a poly- or oligosaccharide via a heterobifunctional compound such as maleimido-hydrazide; (9) protein-lipid conjugation via introducing a hydrophobic group into the protein and (10) protein-lipid conjugation via incorporating a reactive group into the lipid. Also, contemplated are heterobifunctional “non-covalent coupling” techniques such as biotin-avidin interaction. Conjugation techniques are reviewed e.g. in Kalia and Raines, Curr. Org. Chem. (2010) 14 (2): 138-147, which is hereby incorporated by reference in its entirety.

In some embodiments, a payload moiety is attached to a polypeptide of the present disclosure via a linker moiety. Linker moieties for linkage of payload moieties to polypeptides (e.g. polypeptides of antibodies) are known in the art, and are reviewed e.g. in Su et al., Acta Pharm. Sin. B. (2021) 11 (12): 3889-3907, which is hereby incorporated by reference in its entirety. A linker moiety may be a cleavable linker moiety (e.g. an acid cleavable linker moiety), or a non-cleavable linker moiety.

In some embodiments, a payload moiety is attached to a polypeptide of the present disclosure in a non-specific manner.

In some embodiments, the payload moiety is covalently linked to the side chain of an amino acid residue of a polypeptide. In some embodiments, the payload moiety is covalently linked to an amino acid having a side chain comprising an amino group, e.g. lysine. In some embodiments, attachment may be via a peptide bond formed by reaction between the amino group of an amino acid having a side chain comprising an amino group, and an N-hydroxysuccinimide (NHS) moiety of a payload moiety or linker moiety. By way of illustration, in Example 2.2 of the present disclosure, a dye moiety is conjugated to a polypeptide of the disclosure via NHS-lysine conjugation. By way of further illustration, in Example 2.3 of the present disclosure, a horseradish peroxidase moiety is conjugated to a polypeptide of the disclosure via NHS-lysine conjugation.

In some embodiments, a payload moiety is attached to a polypeptide of the present disclosure in a site-specific manner. In some embodiments, a payload moiety is attached to (a) specific amino acid(s) of the polypeptide. In some embodiments, a payload moiety is attached to the N- and/or C-terminus of the polypeptide.

In some embodiments, a payload moiety is provided as a fusion polypeptide with the polypeptide of the present disclosure. That is, an amino acid sequence encoding a payload moiety may be joined to the N- and/or C-terminus of a polypeptide via a peptide bond. By way of illustration, in Example 2.5 of the present disclosure, a GFP moiety is provided as a fusion polypeptide, at the C-terminus of the CH3 domain of a polypeptide of the disclosure.

In some embodiments, a payload moiety is attached via cross-linkage of lysine ¿-amino and glutamine γ-carboxyamide groups. By way of illustration, in Example 2.4 of the present disclosure, a ruthenium moiety linked to a K tag is attached via a transglutaminase-catalysed transamination reaction, to a polypeptide comprising a Q tag at its C-terminus.

In some embodiments, a payload moiety is attached via the action of an enzyme for introducing the payload moiety, e.g. in a site specific fashion. By way of illustration, in Example 2.4 of the present disclosure, biotin ligase is employed to introduce a biotin moiety at the lysine residue of an AviTag provided at the N-terminus of a polypeptide.

In some embodiments, a polypeptide of the present disclosure does not comprise a payload moiety. In some embodiments, a polypeptide complex of the present disclosure does not comprise a payload moiety.

In some embodiments, a polypeptide complex of the present disclosure (e.g. a final, payload-bearing polypeptide complex) comprises (i) a polypeptide comprising a payload moiety, and (ii) a polypeptide not comprising a payload moiety. By way of illustration, in the final payload-bearing polypeptide complex shown in the schematic of FIG. 1 (i.e. the ‘defined, labelled antibody’), one of the polypeptides of the complex (i.e. the polypeptide from the ‘donor’ precursor complex) comprises a payload moiety, and the other polypeptide of the complex (i.e. the polypeptide from the ‘acceptor’ precursor complex) does not comprise a payload moiety.

In some embodiments, a polypeptide or polypeptide complex according to the present disclosure comprises more than one payload moiety. In some embodiments, a polypeptide or polypeptide complex according to the present disclosure comprises one or more (e.g. one of 1, 2, 3, 4 or more) payload moieties. In accordance with such embodiments, each payload moiety may independently be a payload moiety as defined hereinabove.

### Functional Moieties

Aspects and embodiments of the present disclosure relate to polypeptide complexes comprising functional moieties, and polypeptides comprising functional moieties.

Herein, a ‘functional moiety’ refers any moiety having a function. In some embodiments a functional moiety, is or comprises, a binding moiety (e.g. an antibody or a target-binding fragment or derivative thereof, a target-binding peptide/polypeptide, a target-binding nucleic acid), a detectable moiety (e.g. a fluorescent moiety, luminescent moiety, radiopaque/contrast agent, radiolabel, immuno-detectable moieties), a moiety having a detectable activity (e.g. an enzymatic moiety) or a drug moiety (e.g. a cytotoxic moiety).

It will be appreciated that there is considerable overlap between a functional moiety and a payload moiety. In preferred embodiments, in polypeptides and/or polypeptide complexes according to the present disclosure comprising a functional moiety and a payload moiety, the functional moiety and payload moiety are non-identical.

In some embodiments a functional moiety, is or comprises, a binding moiety. Binding moieties include, e.g. target-binding moieties include antibodies (e.g. monoclonal antibodies, e.g. multispecific antibodies), antibody fragments/derivatives (e.g. Fv, scFv, Fab, scFab, F(ab′)2, Fab2, diabodies, triabodies, scFv-Fc, minibodies, single domain antibodies (e.g. VhH), etc.), displaying binding to the relevant target molecule(s). Target-binding moieties include moieties comprising an antigen-binding domain derived from an antibody, e.g. comprising, or consisting of, the Fv (e.g. provided as an scFv) or the Fab region of an antibody, or the whole antibody.

In some embodiments, a target-binding moiety comprises an antibody heavy chain variable region (VH) and an antibody light chain variable region (VL) of an antibody capable of specific binding to a target antigen. The antigen-binding domain formed by a VH and a VL may also be referred to herein as an Fv region. In some embodiments, a target-binding moiety comprises, or consists of, an antigen-binding polypeptide, or an antigen-binding polypeptide complex. An antigen-binding polypeptide complex may comprise more than one polypeptide which together form an antigen-binding domain. The polypeptides of an antigen-binding polypeptide complex may associate covalently or non-covalently. In some embodiments, the polypeptides form part of a larger polypeptide comprising the polypeptides (e.g. in the case of scFv comprising VH and VL, or in the case of scFab comprising VH-CH1 and VL-CL). In some embodiments, the polypeptides are not provided in the same polypeptide (e.g. in the case of a Fab fragment, or an IgG-like moiety).

In some embodiments, a target-binding moiety comprises or consists of an aptamer capable of binding to the target antigen, e.g. a nucleic acid aptamer (reviewed, for example, in Zhou and Rossi, Nat. Rev. Drug. Discov. (2017) 16 (3): 181-202). In some embodiments, a target-binding moiety comprises or consists of a target-binding peptide/polypeptide, e.g. a peptide aptamer, thioredoxin, monobody, anticalin, Kunitz domain, avimer, knottin, fynomer, atrimer, DARPin, affibody, nanobody (i.e. a single-domain antibody (sdAb)), affilin, armadillo repeat protein (ArmRP), OBody or fibronectin—reviewed e.g. in Reverdatto et al., Curr. Top. Med. Chem. (2015) 15 (12): 1082-1101, which is hereby incorporated by reference in its entirety (see also e.g. Boersma et al., J. Biol. Chem. (2011) 286:41273-85 and Emanuel et al., Mabs (2011) 3:38-48).

In some embodiments, a functional moiety is, or comprises, a fluorescent moiety, e.g. as described hereinabove. In some embodiments, a functional moiety is, or comprises, a luminescent moiety, e.g. as described hereinabove. In some embodiments, a functional moiety is, or comprises, a radiopaque or contrast agent, e.g. as described hereinabove. In some embodiments, a functional moiety is, or comprises, a radiolabel, e.g. as described hereinabove. In some embodiments, a functional moiety is, or comprises, an immuno-detectable moiety, e.g. as described hereinabove. In some embodiments, a functional moiety is, or comprises, a moiety having a detectable activity, e.g. an enzymatic moiety, e.g. as described hereinabove. In some embodiments, a functional moiety is, or comprises, a drug moiety, e.g. as described hereinabove.

Functional moieties may be attached/conjugated to a polypeptide of the disclosure (e.g. a polypeptide of a polypeptide complex of the present disclosure) by any suitable means, including those means described hereinabove with respect to payload moieties. In some embodiments, a functional moiety may be covalently linked (e.g. chemically conjugated) to a polypeptide of the present disclosure. In some embodiments, a functional moiety is attached to a polypeptide via a linker moiety.

In some embodiments, a functional moiety is attached to a polypeptide of the present disclosure in a site-specific manner. In some embodiments, a functional moiety is attached to (a) specific amino acid(s) of the polypeptide. In some embodiments, a functional moiety is attached to the N- and/or C-terminus of the polypeptide.

In some embodiments, a functional moiety is provided as a fusion polypeptide with the polypeptide of the present disclosure. That is, an amino acid sequence encoding a functional moiety may be joined to the N- and/or C-terminus of a polypeptide via a peptide bond. By way of illustration, in the molecules of the experimental examples, the functional moiety comprises a Fab fragment, the VH-CHI component of which is provided as a fusion protein, linked to the N-terminus of a CH3 domain-bearing polypeptide of the present disclosure. It will be appreciated that the VL-CL component of the Fab fragment associates by interaction between the VH-CH1 and VL-CL regions.

In some embodiments, a functional moiety is attached to a polypeptide in a non-specific manner (e.g. via covalent linkage to the side chain of an amino acid residue of a polypeptide (e.g. an amino acid having a side chain comprising an amino group, e.g. lysine)). In some embodiments, a functional moiety is attached via a peptide bond formed by reaction of an NHS moiety of a functional moiety or linker moiety, to the amino group of an amino acid residue of a polypeptide.

In some embodiments, a polypeptide of the present disclosure does not comprise a functional moiety. In some embodiments, a polypeptide complex of the present disclosure does not comprise a functional moiety.

In some embodiments, a polypeptide complex of the present disclosure (e.g. a final, functional-bearing polypeptide complex) comprises (i) a polypeptide comprising a functional moiety, and (ii) a polypeptide not comprising a functional moiety. By way of illustration, in the final functional-bearing polypeptide complex shown in the schematic of FIG. 1 (i.e. the ‘defined, labelled antibody’), one of the polypeptides of the complex (i.e. the polypeptide from the ‘acceptor’ precursor complex) comprises a functional moiety (i.e. the Fab region), and the other polypeptide of the complex (i.e. the polypeptide from the ‘donor’ precursor complex) does not comprise a functional moiety.

In some embodiments, a polypeptide or polypeptide complex according to the present disclosure comprises more than one functional moiety. In some embodiments, a polypeptide or polypeptide complex according to the present disclosure comprises one or more (e.g. one of 1, 2, 3, 4 or more) functional moieties. In accordance with such embodiments, each functional moiety may independently be a functional moiety as defined hereinabove.

### Further Domains/Regions of the Polypeptides

In aspects and embodiments of the present disclosure, polypeptides described herein (e.g. constituent polypeptides of polypeptide complexes of the present disclosure) comprise further amino acids/sequences of amino acids, e.g. amino acids/sequences of amino acids forming further peptides/polypeptides/domains.

### CH2 Domains

A polypeptide according to the present disclosure may comprise a CH2 domain. A CH2 domain may be provided upstream of (i.e. N-terminal to) a CH3 domain, in the amino acid sequence of the polypeptide. In such embodiments, a CH2 domain may be provided immediately upstream of the CH3 domain (i.e. adjacent in the amino acid sequence of the polypeptide to the CH3 domain).

Herein, a ‘CH2 domain’ refers to an amino acid sequence corresponding to the CH2 domain of an immunoglobulin (Ig). The CH2 domain is the region of an Ig formed by positions 231 to 340 of the immunoglobulin constant domain, according to the EU numbering system described in Edelman et al., Proc. Natl. Acad. Sci. USA (1969) 63 (1): 78-85.

In some embodiments, the CH2 domain corresponds to or is derived from the CH2 domain of an IgG (e.g. IgG1, IgG2, IgG3, IgG4), IgA (e.g. IgA1, IgA2), IgD, IgE or IgM.

In some embodiments, the CH2 domain corresponds to or is derived from the CH2 domain of a human IgG (e.g. hIgG1, hIgG2, hIgG3, hIgG4), hIgA (e.g. hIgA1, hIgA2), hIgD, hIgE or hIgM. In some embodiments, the CH2 domain corresponds to or is derived from the CH2 domain of a human IgG1 allotype (e.g. Glm1, G1m2, G1m3 or Glm17).

The CH2 domain of the Glml and G1m3 allotypes of human IgG1 is shown in SEQ ID NO: 11. The CH2 domain of human IgG2 is shown in SEQ ID NO:12. The CH2 domain of human IgG3 is shown in SEQ ID NO:13. The CH2 domain of human IgG4 is shown in SEQ ID NO: 14. The second Ig-like domain of human IgA1 is shown in SEQ ID NO:15. The second Ig-like domain of human IgA2 is shown in SEQ ID NO:16. The second Ig-like domain of human IgD is shown in SEQ ID NO:17. The second Ig-like domain of human IgE is shown in SEQ ID NO: 18. The CH2 domain of human IgM is shown in SEQ ID NO:19.

In some embodiments, a CH2 domain according to the present disclosure comprises, or consists of, an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:11, 12, 13, 14, 15, 16, 17, 18 or 19.

In some embodiments, a CH2 domain comprises, or consists of, an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:11, 12, 13 or 14.

In some embodiments, a CH2 domain comprises, or consists of, an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:11.

In aspects and embodiments, a CH2 domain of the present disclosure comprises modification to promote association with another a CH2 domain. In some embodiments, a CH2 domain comprises modification to promote heteromerisation, i.e. association between non-identical CH2 domains.

Where a CH2 region described herein comprises a modification and moreover comprises or consists of an amino acid sequence within a specified threshold percent amino acid sequence identity to a reference amino acid sequence, it will be appreciated that any variation relative to the reference amino acid sequence is confined to positions of the reference sequence other than the modified position(s).

In some embodiments, a CH2 domain of the present disclosure comprises a modification that would, when provided in an Fc region comprising the CH2 domain, increase or decrease the level of an Fc-mediated function (i.e. as compared to the level of the Fc-mediated function displayed by the equivalent Fc region lacking the modification).

In some embodiments, a CH2 domain comprises modification to increase an Fc-mediated function. In some embodiments, a CH2 domain comprises modification to increase ADCC, ADCP and/or CDC. In some embodiments, a CH2 domain comprises modification to increase binding to an Fc receptor (e.g. an Fcγ receptor, e.g. one or more of FcγRI, FcγRIIa, FcγRIIb, FcγRIIc, FcγRIIIa and FcγRIIIb). In some embodiments, a CH2 domain comprises modification to increase binding to FcRn. In some embodiments, a CH2 domain comprises modification to increase binding to a complement protein (e.g. Clq).

In some embodiments, a CH2 domain comprises modification to decrease an Fc-mediated function. In some embodiments, a CH2 domain comprises modification to decrease ADCC, ADCP and/or CDC. In some embodiments, a CH2 domain comprises modification to decrease binding to an Fc receptor (e.g. an Fcγ receptor, e.g. one or more of FcγRI, FcγRIIa, FcγRIIb, FcγRIIc, FcγRIIIa and FcγRIIIb). In some embodiments, a CH2 domain comprises modification to decrease binding to FcRn. In some embodiments, a CH2 domain comprises modification to decrease binding to a complement protein (e.g. Clq).

In embodiments and aspects of the present disclosure, a CH2 domain—e.g. a CH2 domain of a constituent polypeptide of a polypeptide complex according to the present disclosure-comprises a CH2 domain modification known to influence Fc-mediated function described Wang et al., Protein Cell (2018) 9 (1): 63-73.

### Hinge Regions

A polypeptide according to the present disclosure may comprise a hinge region. A hinge region may be provided upstream of (i.e. N-terminal to) a CH2 domain (and a CH3 domain), in the amino acid sequence of the polypeptide. In such embodiments, a hinge region may be provided immediately upstream of the CH2 domain (i.e. adjacent in the amino acid sequence of the polypeptide to the CH2 domain).

Herein, a ‘hinge region’ refers to an amino acid sequence corresponding to the hinge region of an immunoglobulin (Ig). The hinge region is the region of an Ig formed by positions 216 to 230 of the immunoglobulin constant domain, according to the EU numbering system described in Edelman et al., Proc. Natl. Acad. Sci. USA (1969) 63 (1): 78-85.

In some embodiments, the hinge region corresponds to or is derived from the hinge region of an IgG (e.g. IgG1, IgG2, IgG3, IgG4), IgA (e.g. IgA1, IgA2), IgD, IgE or IgM.

In some embodiments, the hinge region corresponds to or is derived from the hinge region of a human IgG (e.g. hIgG1, hIgG2, hIgG3, hIgG4), hIgA (e.g. hIgA1, hIgA2), hIgD, hIgE or hIgM. In some embodiments, the hinge region corresponds to or is derived from the hinge region of a human IgG1 allotype (e.g. Glm1, G1m2, G1m3 or Glm17).

The hinge region of the Glml and G1m3 allotypes of human IgG1 is shown in SEQ ID NO: 20. The hinge region of human IgG2 is shown in SEQ ID NO:21. The hinge region of human IgG3 is shown in SEQ ID NO:22. The hinge region of human IgG4 is shown in SEQ ID NO: 23.

In some embodiments, a hinge region according to the present disclosure comprises, or consists of, an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:20, 21, 22 or 23.

In some embodiments, a hinge region comprises, or consists of, an amino acid sequence having at least 70% amino acid sequence identity, e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity to SEQ ID NO:20.

In some aspects and embodiments, a hinge region of the present disclosure comprises modification to promote association with another hinge region. In some embodiments, a hinge region comprises modification to promote heteromerisation, i.e. association between non-identical hinge regions.

Where a hinge region described herein comprises a modification and moreover comprises or consists of an amino acid sequence within a specified threshold percent amino acid sequence identity to a reference amino acid sequence, it will be appreciated that any variation relative to the reference amino acid sequence is confined to positions of the reference sequence other than the modified position(s).

In some aspects and embodiments, a hinge region of the present disclosure comprises modification to disrupt association with another hinge region. In some embodiments, a hinge region comprises modification to disrupt the formation of a disulfide bond between the hinge region and a hinge region provided on another polypeptide (e.g. in a polypeptide complex comprising two polypeptides, each comprising a hinge region). In some embodiments, a hinge region comprises modification to replace one or more cysteine residues with another amino acid (e.g. one or more cysteine residues participating in the formation of a disulfide bond between the hinge region and a hinge region provided on another polypeptide).

### Linker Sequences

In some embodiments, polypeptides of the present disclosure comprise one or more linker sequences between amino acid sequences. A linker sequence may be provided at one or both ends of one or more of domain(s)/region(s) of a polypeptide described herein, e.g. a CH3 domain, CH2 domain, hinge region, etc.

Linker sequences are known to the skilled person, and are described, for example in Chen et al., Adv. Drug Deliv. Rev. (2013) 65 (10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv. Drug Deliv. Rev. (2013) 65 (10): 1357-1369. Flexible linker sequences often comprise high proportions of glycine and/or serine residues.

In some embodiments, the linker sequence comprises at least one glycine residue and/or at least one serine residue. In some embodiments, the linker sequence comprises or consists of glycine and serine residues.

In some embodiments, the linker sequence has the structure: (GxS) n or (GxS) nGm; wherein G=glycine, S=serine, x=3 or 4, n=2, 3, 4, 5 or 6, and m=0, 1, 2 or 3. In some embodiments, x=3, n=3, 4, 5 or 6, and m=0, 1, 2 or 3; or x=4, n=2, 3, 4 or 5 and m=0, 1, 2 or 3. In some embodiments, x=4 and n=2 or 3, and m=0. In some embodiments, x=4 and n=2.

In some embodiments, the linker sequence comprises one or more (e.g. 1, 2, 3, 4, 5 or 6) copies (e.g. in tandem) of the sequence motif G₄S. In some embodiments, the linker sequence comprises or consists of (G₄S)₄ or (G₄S)₆. In some embodiments, the linker sequence has a length of 1-2, 1-3, 1-4, 1-5, 1-10, 1-15, 1-20, 1-25, or 1-30 amino acids.

### Additional Sequences

Polypeptides of the present disclosure may comprise amino acid sequence(s) to facilitate expression, folding, trafficking, processing, purification, isolation or detection of the polypeptide, or of a polypeptide complex comprising the polypeptide.

In some embodiments, a polypeptide of the present disclosure comprises a tag for facilitating isolation/purification of polypeptides comprising the tag (and/or polypeptide complexes comprising the polypeptide). Suitable tags, which may be referred to as epitope tags, are well known in the art. For example, a polypeptide may comprise a sequence encoding a His, (e.g. (His) 6), c-Myc, GST, MBP, CBP, FLAG, HA, E or C tag. Such tags may be provided at the N- and/or C-terminus of the polypeptide. By way of illustration, a polypeptide of the payload-bearing precursor complexes of the experimental examples comprises a C-tag at its C-terminus.

In some embodiments, a tag may be used to facilitate separation of final, payload-bearing polypeptide complexes from by-product ‘dummy’ polypeptide complexes. In some embodiments, a tag may be used to deplete unreacted precursor polypeptide complexes from the reacted, polypeptide-exchanged complexes. In some embodiments, a polypeptide complex of the present disclosure (e.g. a precursor polypeptide complex) comprises a polypeptide comprising a given tag, and a polypeptide not comprising the tag. In some embodiments, both polypeptides of a polypeptide complex (e.g. by-product ‘dummy’ polypeptide complex) comprise the same tag. In some embodiments, a given tag is provided on polypeptides of polypeptide complexes that are not comprised in the final, payload-bearing polypeptide complex.

By way of illustration, in the method represented schematically in FIG. 1 of the present disclosure, the polypeptides of the precursor ‘donor’ and ‘acceptor’ polypeptide complexes that are not comprised in the final, payload-bearing polypeptide complexes (i.e. the ‘defined labelled antibodies’) each comprise a tag, in this example a C-tag. The C-tag provides for depletion of unreacted precursor ‘donor’ and ‘acceptor’ polypeptide complexes (both of which comprise a polypeptide comprising a C-tag), and also depletion of by-product ‘dummy’ polypeptide complexes (i.e. the ‘dummy dimers’), using an anti-C-tag affinity column. It will be appreciated that following application of a mixture comprising ‘donor’, ‘acceptor’, by-product ‘dummy’ and final, payload-bearing polypeptide complexes to the anti-C-tag affinity column, the flowthrough comprises only those species lacking the C-tag, i.e. the final, payload-bearing polypeptide complexes.

Polypeptides of the present disclosure may comprise amino acid sequence(s) to facilitate attachment of a payload moiety and/or a functional moiety. For example, a polypeptide may comprise a sequence encoding a K tag (e.g. (Lys) 6), a Q tag (e.g. Trp-Leu-Ala-Gln-Arg-Pro-His) or AviTag. By way of illustration, in Example 2.4 of the present disclosure, a ruthenium moiety is attached to a polypeptide comprising a Q tag at its C-terminus via the action of transglutaminase on the K tag-linked ruthenium. By way of further illustration, in Example 2.4 of the present disclosure, a polypeptide comprises AviTag at the N-terminus, for site-specific addition of a biotin using the E. coli biotin ligase BirA.

Polypeptides of the present disclosure may comprise a signal peptide (also known as a leader sequence or signal sequence). Signal peptides normally consist of a sequence of 5-30 hydrophobic amino acids, which form a single alpha helix. Secreted proteins and proteins expressed at the cell surface often comprise signal peptides. The signal peptide may be present at the N-terminus of the polypeptide, and may be present in the newly-synthesised polypeptide. The signal peptide may provide for efficient trafficking and secretion of the polypeptide. Signal peptides are often removed by cleavage (e.g. by signal peptidases), and so may not be comprised in the final, mature form of the polypeptide secreted from a cell expressing the polypeptide.

Signal peptides are known for many proteins, and are recorded in databases such as GenBank, UniProt, Swiss-Prot, TrEMBL, Protein Information Resource, Protein Data Bank, Ensembl, and InterPro, and/or can be identified/predicted e.g. using amino acid sequence analysis tools such as SignalP (Petersen et al., Nature Methods (2011) 8:785-786) or Signal-BLAST (Frank and Sippl, Bioinformatics (2008) 24:2172-2176).

In some embodiments, polypeptides according to the present disclosure may comprise one or more additional amino acids (e.g. 1-5, 1-10, 1-20, 1-30, 1-40, 1-50, 5-10, 5-20, 5-30, 5-40, 5-50, 10-35 20, 10-30, 10-40, 10-50, 20-30, 20-40 or 20-50 additional amino acids) at the N- and/or C-terminus of the polypeptide.

### Particular Exemplary Polypeptides and Polypeptide Complexes

In some embodiments, polypeptides (e.g. constituent polypeptides of polypeptide complexes) according to the present disclosure comprise a CH3 domain and a CH2 domain. In such embodiments, the CH2 domain may be provided upstream of (i.e. N-terminal to) the CH3 domain, in the amino acid sequence of the polypeptide. In such embodiments, the CH2 domain may be provided immediately upstream of the CH3 domain (i.e. adjacent in the amino acid sequence of the polypeptide to the CH3 domain). In some embodiments, polypeptides according to the present disclosure comprise the structure: N term-[ . . . ]-[CH2 domain]-[CH3 domain]-[ . . . ]-C term.

In some embodiments, polypeptides (e.g. constituent polypeptides of polypeptide complexes) according to the present disclosure comprise a hinge region. In such embodiments, the hinge region may be provided upstream of (i.e. N-terminal to) the CH3 domain, in the amino acid sequence of the polypeptide. In embodiments wherein polypeptides comprise a CH2 domain, the hinge region may be provided upstream of (i.e. N-terminal to) the CH2 domain, in the amino acid sequence of the polypeptide. In such embodiments, the hinge region may be provided immediately upstream of the CH2 domain (i.e. adjacent in the amino acid sequence of the polypeptide to the CH2 domain).

In some embodiments, polypeptides (e.g. constituent polypeptides of polypeptide complexes) according to the present disclosure comprise a hinge region, a CH2 domain and a CH3 domain. In some embodiments, polypeptides according to the present disclosure comprise the structure: N term-[ . . . ]-[hinge region]-[CH2 domain]-[CH3 domain]-[ . . . ]-C term.

The following particular exemplary polypeptides are contemplated in connection with the present disclosure:

(A) A polypeptide comprising a CH3 domain having at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to an amino acid sequence according to any one of SEQ ID NOs: 24 to 47.

(B) A polypeptide according to (A), further comprising a payload moiety.

(C) A polypeptide according to (A) or (B), further comprising a CH2 domain, optionally wherein the CH2 domain is N-terminal to the CH3 domain.

(D) A polypeptide according to (C), further comprising a hinge region, optionally wherein the hinge region is N-terminal to the CH2 domain.

(E) A polypeptide comprising according to any one of (A) to (D), wherein the polypeptide comprises (e.g. in the order (i)=most N-terminal, and (iii)=C-terminal, in the amino acid sequence of the polypeptide):
(i) a hinge region;
(ii) a CH3 domain; and
(iii) a CH3 domain having at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to an amino acid sequence according to any one of SEQ ID NOs: 24 to 47.

The following particular exemplary polypeptide complexes are contemplated in connection with the present disclosure:

### Precursor Polypeptide Complexes

(1) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide comprises a knob modification, and the modification(s) from column A of one of rows 1 to 49 of Table II; and
wherein the CH3 domain of the second polypeptide comprises a hole modification, and the modification(s) from column B of the same row of Table II that the modification(s) from column A are selected.

By way of illustration, a polypeptide complex according to the present disclosure according to (1) above may comprise: (i) a first polypeptide comprising a CH3 domain comprising 366W (i.e. knob modification), 370E and 439E (i.e. the modifications of row 1 of column A of Table II), and (ii) a second polypeptide comprising a CH3 domain comprising 407V, 366S, 368A (i.e. hole modification) and 356K (i.e. the modification of row 1 of column B of Table II).

(2) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide comprises a knob modification, and the modification(s) from column A of one of rows 1 to 12 of Table III; and
wherein the CH3 domain of the second polypeptide comprises a hole modification, and the modification(s) from column B of the same row of Table III that the modification(s) from column A are selected.

(3) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide comprises a knob modification, and the modification(s) from column A of one of rows 1 to 14 of Table IV; and
wherein the CH3 domain of the second polypeptide comprises a hole modification, and the modification(s) from column B of the same row of Table IV that the modification(s) from column A are selected.

(4) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide comprises a knob modification, and the modification(s) from column A of one of rows 1 to 8 of Table V; and
wherein the CH3 domain of the second polypeptide comprises a hole modification, and the modification(s) from column B of the same row of Table V that the modification(s) from column A are selected.

(5) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide comprises a knob modification, and the modification(s) from column A of one of rows 1 to 18 of Table VI; and
wherein the CH3 domain of the second polypeptide comprises a hole modification, and the modification(s) from column B of the same row of Table VI that the modification(s) from column A are selected.

(6) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide comprises a knob modification, and the modification(s) from column A of one of rows 1 to 24 of Table VII; and
wherein the CH3 domain of the second polypeptide comprises a hole modification, and the modification(s) from column B of the same row of Table VII that the modification(s) from column A are selected.

(7) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide comprises 366W and 370E; and
wherein the CH3 domain of the second polypeptide comprises 407V, 366S, and 368A.

(8) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide comprises 366W; and
wherein the CH3 domain of the second polypeptide comprises 407V, 366S, 368A and 357K.

(9) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide comprises 366W, 370E and 354C; and
wherein the CH3 domain of the second polypeptide comprises 407V, 366S, and 368A.

(10) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide comprises 366W; and
wherein the CH3 domain of the second polypeptide comprises 407V, 366S, 368A, 357K and 349C.

(11) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide comprises 366W and 370E; and
wherein the CH3 domain of the second polypeptide comprises 407V, 366S, 368A and 349C.

(12) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide comprises 366W and 354C; and
wherein the CH3 domain of the second polypeptide comprises 407V, 366S, 368A, and 357K.

(13) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:24; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:49.

(14) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:48; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:25.

(15) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:26; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:49.

(16) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:48; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:27.

(17) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:24; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:51.

(18) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:50; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:25.

(19) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:28; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:53.

(20) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:52; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:29.

(21) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:30; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:53.

(22) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:52; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:31.

(23) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:28; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:55.

(24) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:54; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:29.

(25) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:32; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:57.

(26) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:56; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:33.

(27) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:34; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:57.

(28) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:56; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:35. (29) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:32; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:59.

(30) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:58; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:33.

(31) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:36; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:61.

(32) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:60; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:37.

(33) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:38; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:61.

(34) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:60; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:39.

(35) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:36; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:63.

(36) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:62; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:37.

(37) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:40; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:65.

(38) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:64; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:41.

(39) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:42; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:65.

(40) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:64; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:43.

(41) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:40; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:67.

(42) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:66; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:41.

(43) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:44; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:69.

(44) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:68; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:45.

(45) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:46; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:69.

(46) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:68; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:47.

(47) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:44; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:71.

(48) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:70; and
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:45.

(49) The polypeptide complex according to any one of (1) to (48), wherein the first polypeptide and/or the second polypeptide further comprises a payload moiety.

(50) The polypeptide complex according to any one of (1) to (49), wherein the first polypeptide and/or the second polypeptide further comprises a functional moiety.

### Final, Payload-Bearing Polypeptide Complexes

(51) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide comprises 366W and 370E;
wherein the CH3 domain of the second polypeptide comprises 407V, 366S, 368A and 357K; and
wherein the first polypeptide or the second polypeptide further comprises a payload moiety.

(52) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide comprises 366W, 370E and 354C;
wherein the CH3 domain of the second polypeptide comprises 407V, 366S, 368A, 357K and 349C; and
wherein the first polypeptide or the second polypeptide further comprises a payload moiety.

(53) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:24;
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:25; and
wherein the first polypeptide or the second polypeptide further comprises a payload moiety.

(54) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:26;
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:27; and
wherein the first polypeptide or the second polypeptide further comprises a payload moiety.

(55) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:28;
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:29; and
wherein the first polypeptide or the second polypeptide further comprises a payload moiety.

(56) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:30;
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:31; and
wherein the first polypeptide or the second polypeptide further comprises a payload moiety.

(57) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:32;
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:33; and
wherein the first polypeptide or the second polypeptide further comprises a payload moiety.

(58) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:34;
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:35; and
wherein the first polypeptide or the second polypeptide further comprises a payload moiety.

(59) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:36;
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:37; and
wherein the first polypeptide or the second polypeptide further comprises a payload moiety.

(60) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:38;
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:39; and
wherein the first polypeptide or the second polypeptide further comprises a payload moiety.

(61) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:40;
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:41; and
wherein the first polypeptide or the second polypeptide further comprises a payload moiety.

(62) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:42;
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:43; and
wherein the first polypeptide or the second polypeptide further comprises a payload moiety.

(63) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:44;
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:45; and
wherein the first polypeptide or the second polypeptide further comprises a payload moiety.

(64) A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:46;
wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:47; and
wherein the first polypeptide or the second polypeptide further comprises a payload moiety.

### Methods for Producing Polypeptide Complexes

Aspects and embodiments of the present disclosure relate to methods for producing polypeptide complexes, particularly payload-bearing polypeptide complexes.

In some aspects and embodiments, a method of the present disclosure may be referred to as a method for producing a payload-bearing polypeptide complex. In some aspects and embodiments, a method of the present disclosure may be referred to as a method for providing a polypeptide with (e.g. labelling a polypeptide with) a payload.

The methods generally comprise contacting a first polypeptide complex comprising a first polypeptide and a second polypeptide with a second polypeptide complex comprising a third polypeptide and a fourth polypeptide, to form a third polypeptide complex comprising the first polypeptide and the fourth polypeptide, and/or a fourth polypeptide complex comprising the second polypeptide and the third polypeptide.

The first polypeptide complex and the second polypeptide complex are contacted with one another under conditions suitable for exchange of their constituent polypeptides, for the formation of the third polypeptide complex and/or the fourth polypeptide complex. In some embodiments, the method is performed essentially as described in Example 1.3 of the present disclosure.

The methods may comprise mixing appropriately equimolar amounts of the first polypeptide complex and the second polypeptide complex. It will be appreciated that first polypeptide complex and the second polypeptide complex are provided at a concentration providing for efficient exchange of their constituent polypeptides. In some embodiments, equimolar amounts of the first polypeptide complex and the second polypeptide complex are mixed at a concentration of 0.1 to 10 mg/ml, e.g. 0.5 to 2 mg/ml (e.g. ˜ 1 mg/ml).

The first polypeptide complex and the second polypeptide complex may be contacted with one another in a suitable buffer. As used herein, a “buffer” refers to a buffered solution that resists changes in pH by the action of its acid-base conjugate components. In some embodiments, a buffer is or comprises phosphate-buffered saline (PBS), pH 7.4.

In some embodiments (e.g. embodiments wherein the first polypeptide complex and/or the second polypeptide complex is formed by interaction between constituent polypeptides comprising one or more interchain disulfide bonds, e.g. formed between cysteine residues of hinge regions of the polypeptides), the methods may comprise treatment of the first polypeptide complex and/or the second polypeptide complex with a reducing agent. The reducing agent may disrupt disulfide bonds of the treated polypeptide complexes. In some embodiments, a reducing agent is tris(2-carboxyethyl) phosphine (TCEP). In some embodiments, the reducing agent is provided to the mixture of the first polypeptide complex and the second polypeptide complex at a molar excess, e.g. a 2-fold to 50-fold molar excess, e.g. a 10-fold to 30-fold molar excess or a 15-fold to 25-fold (e.g. ˜ 20-fold) molar excess.

In some embodiments (e.g. embodiments wherein the first polypeptide complex and/or the second polypeptide complex is formed by interaction between constituent polypeptides not comprising interchain disulfide bonds), the methods may not comprise treatment of the first polypeptide complex and/or the second polypeptide complex with a reducing agent.

The mixture comprising the first polypeptide complex and the second polypeptide complex is incubated at a temperature providing favourable kinetics for chain exchange of their constituent polypeptides. In some embodiments, the mixture comprising the first polypeptide complex and the second polypeptide complex is incubated at a temperature of 4° C. to 60° C., e.g. 15° C. to 40° C. or 25° C. to 40° C. (e.g. ˜ 37° C.).

The mixture comprising the first polypeptide complex and the second polypeptide complex is incubated for a period of time sufficient for chain exchange of their constituent polypeptides to occur. In some embodiments, the mixture comprising the first polypeptide complex and the second polypeptide complex is incubated for 30 min to 12 hours, e.g. 1 hour to 6 hours or 2 hours to 4 hours (e.g. ˜ 3 hours).

Incubation of the mixture comprising the first polypeptide complex and the second polypeptide complex may be performed with agitation, to facilitate mixture. In some embodiments, the mixture comprising the first polypeptide complex and the second polypeptide complex is incubated with agitation at 100 rpm to 600 rpm, e.g. 150 rpm to 500 rpm or 200 rpm to 400 rpm (e.g. ˜ 300 rpm).

The methods of the present disclosure comprise recovering the third polypeptide complex and/or the fourth polypeptide complex, following incubation of the first polypeptide complex and the second polypeptide complex. In some embodiments, ‘recovering’ a polypeptide complex comprises separating/isolating/purifying and/or collecting the polypeptide complex.

In some embodiments, the methods of the present disclosure comprise separating/isolating/purifying a polypeptide complex. For example, following incubation of the first polypeptide complex and the second polypeptide complex for polypeptide chain exchange, products of the reaction may be isolated/purified/depleted. In some embodiments, the methods of the present disclosure comprise separating/isolating/purifying a polypeptide complex by affinity chromatography. Affinity chromatography is well known in the art, and its principles are described e.g. in Rodriguez et al., J. Chromatogr. B. Analyt. Technol. Biomed. Life Sci. (2020) 1157:122332, which is hereby incorporated by reference in its entirety.

In some embodiments, the methods of the present disclosure comprise contacting a mixture comprising the first, second, third and fourth polypeptide complexes with an agent for facilitating separation of one or more of the polypeptide complexes from the mixture.

In embodiments wherein constituent polypeptides of the first polypeptide complex and/or the second polypeptide complex comprise an appropriate tag, the third polypeptide complex and/or the fourth polypeptide complex may be isolated/purified/depleted using an agent for affinity purification of polypeptide complexes comprising the tag. Alternatively, or in addition, when constituent polypeptides of the first polypeptide complex and/or the second polypeptide complex comprise an appropriate tag, unreacted precursor polypeptide complexes may be isolated/purified/depleted from the third polypeptide complex and/or the fourth polypeptide complex using an agent for affinity purification of such precursor polypeptide complexes comprising the tag.

By way of illustration, in the method represented schematically in FIG. 1 of the present disclosure, the polypeptides of the first and second polypeptide complexes (i.e. the precursor ‘donor’ and ‘acceptor’ polypeptide complexes) that are not comprised in the final, payload-bearing polypeptide complexes (i.e. the ‘defined labelled antibodies’) each comprise a tag, in this example a C-tag. The C-tag provides for depletion of unreacted precursor ‘donor’ and ‘acceptor’ polypeptide complexes (both of which comprise a polypeptide comprising a C-tag), and also depletion of by-product ‘dummy’ polypeptide complexes (i.e. the ‘dummy dimers’), using an anti-C-tag affinity column. Following application of a mixture comprising ‘donor’, ‘acceptor’, by-product ‘dummy’ and final, payload-bearing polypeptide complexes to the anti-C-tag affinity column, the flowthrough comprises only those species lacking the C-tag, i.e. the final, payload-bearing polypeptide complexes.

In the example of the preceding paragraph, an agent for affinity purification of polypeptide complexes comprising the tag (i.e. the anti-C-tag affinity column) is employed for the isolation/purification of final, payload-bearing polypeptide complexes by depletion of unreacted precursor ‘donor’ and ‘acceptor’ polypeptide complexes, and of by-product ‘dummy’ polypeptide complexes. However, in alternative embodiments, an agent may be employed to isolate/purify final, payload-bearing polypeptide complexes by positive selection.

In some embodiments, an agent for facilitating separation of one or more of the polypeptide complexes from a mixture comprising the first, second, third and fourth polypeptide complexes comprises a moiety for binding to a tag (i.e. a tag comprised in a polypeptide of one or more of the polypeptide complexes). In some embodiments, a tag may be an epitope tag, e.g. as described herein. In some embodiments, a moiety for binding to a tag may be or comprise an antibody specific for the tag, or an antigen-binding fragment/derivative thereof. In some embodiments, a moiety for binding to a tag may be provided on a solid substrate, e.g. a bead (e.g. an agarose bead). In some embodiments, beads comprising a moiety for binding to a tag may be provided in a column.

Separation/isolation/purification of one or more polypeptide complexes in accordance with the present disclosure may alternatively, or additionally, employ further techniques for separating/isolating/purifying polypeptide complexes. Such techniques include e.g. size-exclusion chromatography, capillary electrophoresis and ion exchange chromatography. In some embodiments, the methods of the present disclosure comprise separating/isolating/purifying polypeptide complexes by size-exclusion chromatography and/or capillary electrophoresis.

### Particular Exemplary Methods for Producing Payload-Bearing Polypeptide Complexes

The following particular exemplary methods for producing polypeptide payload-bearing polypeptide complexes according to the present disclosure are contemplated:

(1) A method for producing a polypeptide complex, comprising:
incubating:
   (i) a first polypeptide complex, comprising a first polypeptide comprising a CH3 domain and a second polypeptide comprising a CH3 domain;
   wherein the first polypeptide complex is formed by interaction between the first polypeptide and the second polypeptide, the interaction comprising association between the CH3 domain of the first polypeptide and the CH3 domain of the second polypeptide;
   wherein the CH3 domain of the first polypeptide comprises 366W and 370E;
   wherein the CH3 domain of the second polypeptide comprises 407V, 366S, and 368A;
and
   (ii) a second polypeptide complex, comprising a third polypeptide comprising a CH3 domain and a fourth polypeptide comprising a CH3 domain;
   wherein the second polypeptide complex is formed by interaction between the third polypeptide and the fourth polypeptide, the interaction comprising association between the CH3 domain of the third polypeptide and the CH3 domain of the fourth polypeptide;
   wherein the CH3 domain of the third polypeptide comprises 366W;
   wherein the CH3 domain of the fourth polypeptide comprises 407V, 366S, 368A and 357K;
to form a third polypeptide complex comprising the first polypeptide and the fourth polypeptide (i.e. a polypeptide complex according to (51) above), and/or a fourth polypeptide complex comprising the second polypeptide and the third polypeptide; and
recovering the third polypeptide complex and/or the fourth polypeptide complex;
wherein the first and/or second polypeptide of the first polypeptide complex or the first and/or second polypeptide of the second polypeptide complex further comprise a payload moiety.

(2) A method for producing a polypeptide complex, comprising:
incubating:
   (i) a first polypeptide complex, comprising a first polypeptide comprising a CH3 domain and a second polypeptide comprising a CH3 domain;
   wherein the first polypeptide complex is formed by interaction between the first polypeptide and the second polypeptide, the interaction comprising association between the CH3 domain of the first polypeptide and the CH3 domain of the second polypeptide;
   wherein the CH3 domain of the first polypeptide comprises 366W, 370E and 354C; and
   wherein the CH3 domain of the second polypeptide comprises 407V, 366S, and 368A;
and
   (ii) a second polypeptide complex, comprising a third polypeptide comprising a CH3 domain and a fourth polypeptide comprising a CH3 domain;
   wherein the second polypeptide complex is formed by interaction between the third polypeptide and the fourth polypeptide, the interaction comprising association between the CH3 domain of the third polypeptide and the CH3 domain of the fourth polypeptide;
   wherein the CH3 domain of the third polypeptide comprises 366W; and
   wherein the CH3 domain of the fourth polypeptide comprises 407V, 366S, 368A, 357K and 349C;
to form a third polypeptide complex comprising the first polypeptide and the fourth polypeptide (i.e. a polypeptide complex according to (52) above), and/or a fourth polypeptide complex comprising the second polypeptide and the third polypeptide; and
recovering the third polypeptide complex and/or the fourth polypeptide complex;
wherein the first and/or second polypeptide of the first polypeptide complex or the first and/or second polypeptide of the second polypeptide complex further comprise a payload moiety.

(3) A method for producing a polypeptide complex, comprising:
incubating:
   (i) a first polypeptide complex, comprising a first polypeptide comprising a CH3 domain and a second polypeptide comprising a CH3 domain;
   wherein the first polypeptide complex is formed by interaction between the first polypeptide and the second polypeptide, the interaction comprising association between the CH3 domain of the first polypeptide and the CH3 domain of the second polypeptide;
   wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:24; and
   wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:49;
and
   (ii) a second polypeptide complex, comprising a third polypeptide comprising a CH3 domain and a fourth polypeptide comprising a CH3 domain;
   wherein the second polypeptide complex is formed by interaction between the third polypeptide and the fourth polypeptide, the interaction comprising association between the CH3 domain of the third polypeptide and the CH3 domain of the fourth polypeptide;
   wherein the CH3 domain of the third polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:48; and
   wherein the CH3 domain of the fourth polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:25;
to form a third polypeptide complex comprising the first polypeptide and the fourth polypeptide (i.e. a polypeptide complex according to (53) above), and/or a fourth polypeptide complex comprising the second polypeptide and the third polypeptide; and
recovering the third polypeptide complex and/or the fourth polypeptide complex;
wherein the first and/or second polypeptide of the first polypeptide complex or the first and/or second polypeptide of the second polypeptide complex further comprise a payload moiety.

(4) A method for producing a polypeptide complex, comprising:
incubating:
   (i) a first polypeptide complex, comprising a first polypeptide comprising a CH3 domain and a second polypeptide comprising a CH3 domain;
   wherein the first polypeptide complex is formed by interaction between the first polypeptide and the second polypeptide, the interaction comprising association between the CH3 domain of the first polypeptide and the CH3 domain of the second polypeptide;
   wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:26; and
   wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:49;
and
   (ii) a second polypeptide complex, comprising a third polypeptide comprising a CH3 domain and a fourth polypeptide comprising a CH3 domain;
   wherein the second polypeptide complex is formed by interaction between the third polypeptide and the fourth polypeptide, the interaction comprising association between the CH3 domain of the third polypeptide and the CH3 domain of the fourth polypeptide;
   wherein the CH3 domain of the third polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:48; and
   wherein the CH3 domain of the fourth polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:27;
to form a third polypeptide complex comprising the first polypeptide and the fourth polypeptide (i.e. a polypeptide complex according to (54) above), and/or a fourth polypeptide complex comprising the second polypeptide and the third polypeptide; and
recovering the third polypeptide complex and/or the fourth polypeptide complex;
wherein the first and/or second polypeptide of the first polypeptide complex or the first and/or second polypeptide of the second polypeptide complex further comprise a payload moiety.

(5) A method for producing a polypeptide complex, comprising:
incubating:
   (i) a first polypeptide complex, comprising a first polypeptide comprising a CH3 domain and a second polypeptide comprising a CH3 domain;
   wherein the first polypeptide complex is formed by interaction between the first polypeptide and the second polypeptide, the interaction comprising association between the CH3 domain of the first polypeptide and the CH3 domain of the second polypeptide;
   wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:28; and
   wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:53;
and
   (ii) a second polypeptide complex, comprising a third polypeptide comprising a CH3 domain and a fourth polypeptide comprising a CH3 domain;
   wherein the second polypeptide complex is formed by interaction between the third polypeptide and the fourth polypeptide, the interaction comprising association between the CH3 domain of the third polypeptide and the CH3 domain of the fourth polypeptide;
   wherein the CH3 domain of the third polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:52; and
   wherein the CH3 domain of the fourth polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:29;
to form a third polypeptide complex comprising the first polypeptide and the fourth polypeptide (i.e. a polypeptide complex according to (55) above), and/or a fourth polypeptide complex comprising the second polypeptide and the third polypeptide; and
recovering the third polypeptide complex and/or the fourth polypeptide complex;
wherein the first and/or second polypeptide of the first polypeptide complex or the first and/or second polypeptide of the second polypeptide complex further comprise a payload moiety.

(5) A method for producing a polypeptide complex, comprising:
incubating:
   (i) a first polypeptide complex, comprising a first polypeptide comprising a CH3 domain and a second polypeptide comprising a CH3 domain;
   wherein the first polypeptide complex is formed by interaction between the first polypeptide and the second polypeptide, the interaction comprising association between the CH3 domain of the first polypeptide and the CH3 domain of the second polypeptide;
   wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:30; and
   wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:53;
and
   (ii) a second polypeptide complex, comprising a third polypeptide comprising a CH3 domain and a fourth polypeptide comprising a CH3 domain;
   wherein the second polypeptide complex is formed by interaction between the third polypeptide and the fourth polypeptide, the interaction comprising association between the CH3 domain of the third polypeptide and the CH3 domain of the fourth polypeptide;
   wherein the CH3 domain of the third polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:52; and
   wherein the CH3 domain of the fourth polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:31;
to form a third polypeptide complex comprising the first polypeptide and the fourth polypeptide (i.e. a polypeptide complex according to (56) above), and/or a fourth polypeptide complex comprising the second polypeptide and the third polypeptide; and
recovering the third polypeptide complex and/or the fourth polypeptide complex;
wherein the first and/or second polypeptide of the first polypeptide complex or the first and/or second polypeptide of the second polypeptide complex further comprise a payload moiety.

(7) A method for producing a polypeptide complex, comprising:
incubating:
   (i) a first polypeptide complex, comprising a first polypeptide comprising a CH3 domain and a second polypeptide comprising a CH3 domain;
   wherein the first polypeptide complex is formed by interaction between the first polypeptide and the second polypeptide, the interaction comprising association between the CH3 domain of the first polypeptide and the CH3 domain of the second polypeptide;
   wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:32; and
   wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:57;
and
   (ii) a second polypeptide complex, comprising a third polypeptide comprising a CH3 domain and a fourth polypeptide comprising a CH3 domain;
   wherein the second polypeptide complex is formed by interaction between the third polypeptide and the fourth polypeptide, the interaction comprising association between the CH3 domain of the third polypeptide and the CH3 domain of the fourth polypeptide;
   wherein the CH3 domain of the third polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:56; and
   wherein the CH3 domain of the fourth polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:33;
to form a third polypeptide complex comprising the first polypeptide and the fourth polypeptide (i.e. a polypeptide complex according to (57) above), and/or a fourth polypeptide complex comprising the second polypeptide and the third polypeptide; and
recovering the third polypeptide complex and/or the fourth polypeptide complex;
wherein the first and/or second polypeptide of the first polypeptide complex or the first and/or second polypeptide of the second polypeptide complex further comprise a payload moiety.

(8) A method for producing a polypeptide complex, comprising:
incubating:
   (i) a first polypeptide complex, comprising a first polypeptide comprising a CH3 domain and a second polypeptide comprising a CH3 domain;
   wherein the first polypeptide complex is formed by interaction between the first polypeptide and the second polypeptide, the interaction comprising association between the CH3 domain of the first polypeptide and the CH3 domain of the second polypeptide;
   wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:34; and
   wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:57;
and
   (ii) a second polypeptide complex, comprising a third polypeptide comprising a CH3 domain and a fourth polypeptide comprising a CH3 domain;
   wherein the second polypeptide complex is formed by interaction between the third polypeptide and the fourth polypeptide, the interaction comprising association between the CH3 domain of the third polypeptide and the CH3 domain of the fourth polypeptide;
   wherein the CH3 domain of the third polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:56; and
   wherein the CH3 domain of the fourth polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:35;
to form a third polypeptide complex comprising the first polypeptide and the fourth polypeptide (i.e. a polypeptide complex according to (58) above), and/or a fourth polypeptide complex comprising the second polypeptide and the third polypeptide; and
recovering the third polypeptide complex and/or the fourth polypeptide complex;
wherein the first and/or second polypeptide of the first polypeptide complex or the first and/or second polypeptide of the second polypeptide complex further comprise a payload moiety.

(9) A method for producing a polypeptide complex, comprising:
incubating:
   (i) a first polypeptide complex, comprising a first polypeptide comprising a CH3 domain and a second polypeptide comprising a CH3 domain;
   wherein the first polypeptide complex is formed by interaction between the first polypeptide and the second polypeptide, the interaction comprising association between the CH3 domain of the first polypeptide and the CH3 domain of the second polypeptide;
   wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:36; and
   wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:61;
and
   (ii) a second polypeptide complex, comprising a third polypeptide comprising a CH3 domain and a fourth polypeptide comprising a CH3 domain;
   wherein the second polypeptide complex is formed by interaction between the third polypeptide and the fourth polypeptide, the interaction comprising association between the CH3 domain of the third polypeptide and the CH3 domain of the fourth polypeptide;
   wherein the CH3 domain of the third polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:60; and
   wherein the CH3 domain of the fourth polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:37;
to form a third polypeptide complex comprising the first polypeptide and the fourth polypeptide (i.e. a polypeptide complex according to (59) above), and/or a fourth polypeptide complex comprising the second polypeptide and the third polypeptide; and
recovering the third polypeptide complex and/or the fourth polypeptide complex;
wherein the first and/or second polypeptide of the first polypeptide complex or the first and/or second polypeptide of the second polypeptide complex further comprise a payload moiety.

(10) A method for producing a polypeptide complex, comprising:
incubating:
   (i) a first polypeptide complex, comprising a first polypeptide comprising a CH3 domain and a second polypeptide comprising a CH3 domain;
   wherein the first polypeptide complex is formed by interaction between the first polypeptide and the second polypeptide, the interaction comprising association between the CH3 domain of the first polypeptide and the CH3 domain of the second polypeptide;
   wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:38; and
   wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:61;
and
   (ii) a second polypeptide complex, comprising a third polypeptide comprising a CH3 domain and a fourth polypeptide comprising a CH3 domain;
   wherein the second polypeptide complex is formed by interaction between the third polypeptide and the fourth polypeptide, the interaction comprising association between the CH3 domain of the third polypeptide and the CH3 domain of the fourth polypeptide;
   wherein the CH3 domain of the third polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:60; and
   wherein the CH3 domain of the fourth polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:39;
to form a third polypeptide complex comprising the first polypeptide and the fourth polypeptide (i.e. a polypeptide complex according to (60) above), and/or a fourth polypeptide complex comprising the second polypeptide and the third polypeptide; and
recovering the third polypeptide complex and/or the fourth polypeptide complex;
wherein the first and/or second polypeptide of the first polypeptide complex or the first and/or second polypeptide of the second polypeptide complex further comprise a payload moiety.

(11) A method for producing a polypeptide complex, comprising:
incubating:
   (i) a first polypeptide complex, comprising a first polypeptide comprising a CH3 domain and a second polypeptide comprising a CH3 domain;
   wherein the first polypeptide complex is formed by interaction between the first polypeptide and the second polypeptide, the interaction comprising association between the CH3 domain of the first polypeptide and the CH3 domain of the second polypeptide;
   wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:40; and
   wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:65;
and
   (ii) a second polypeptide complex, comprising a third polypeptide comprising a CH3 domain and a fourth polypeptide comprising a CH3 domain;
   wherein the second polypeptide complex is formed by interaction between the third polypeptide and the fourth polypeptide, the interaction comprising association between the CH3 domain of the third polypeptide and the CH3 domain of the fourth polypeptide;
   wherein the CH3 domain of the third polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:64; and
   wherein the CH3 domain of the fourth polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:41;
to form a third polypeptide complex comprising the first polypeptide and the fourth polypeptide (i.e. a polypeptide complex according to (61) above), and/or a fourth polypeptide complex comprising the second polypeptide and the third polypeptide; and
recovering the third polypeptide complex and/or the fourth polypeptide complex;
wherein the first and/or second polypeptide of the first polypeptide complex or the first and/or second polypeptide of the second polypeptide complex further comprise a payload moiety.

(12) A method for producing a polypeptide complex, comprising:
incubating:
   (i) a first polypeptide complex, comprising a first polypeptide comprising a CH3 domain and a second polypeptide comprising a CH3 domain;
   wherein the first polypeptide complex is formed by interaction between the first polypeptide and the second polypeptide, the interaction comprising association between the CH3 domain of the first polypeptide and the CH3 domain of the second polypeptide;
   wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:42; and
   wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:65;
and
   (ii) a second polypeptide complex, comprising a third polypeptide comprising a CH3 domain and a fourth polypeptide comprising a CH3 domain;
   wherein the second polypeptide complex is formed by interaction between the third polypeptide and the fourth polypeptide, the interaction comprising association between the CH3 domain of the third polypeptide and the CH3 domain of the fourth polypeptide;
   wherein the CH3 domain of the third polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:64; and
   wherein the CH3 domain of the fourth polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:43;
to form a third polypeptide complex comprising the first polypeptide and the fourth polypeptide (i.e. a polypeptide complex according to (62) above), and/or a fourth polypeptide complex comprising the second polypeptide and the third polypeptide; and
recovering the third polypeptide complex and/or the fourth polypeptide complex;
wherein the first and/or second polypeptide of the first polypeptide complex or the first and/or second polypeptide of the second polypeptide complex further comprise a payload moiety.

(13) A method for producing a polypeptide complex, comprising:
incubating:
   (i) a first polypeptide complex, comprising a first polypeptide comprising a CH3 domain and a second polypeptide comprising a CH3 domain;
   wherein the first polypeptide complex is formed by interaction between the first polypeptide and the second polypeptide, the interaction comprising association between the CH3 domain of the first polypeptide and the CH3 domain of the second polypeptide;
   wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:44; and
   wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:69;
and
   (ii) a second polypeptide complex, comprising a third polypeptide comprising a CH3 domain and a fourth polypeptide comprising a CH3 domain;
   wherein the second polypeptide complex is formed by interaction between the third polypeptide and the fourth polypeptide, the interaction comprising association between the CH3 domain of the third polypeptide and the CH3 domain of the fourth polypeptide;
   wherein the CH3 domain of the third polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:68; and
   wherein the CH3 domain of the fourth polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:45;
to form a third polypeptide complex comprising the first polypeptide and the fourth polypeptide (i.e. a polypeptide complex according to (63) above), and/or a fourth polypeptide complex comprising the second polypeptide and the third polypeptide; and
recovering the third polypeptide complex and/or the fourth polypeptide complex;
wherein the first and/or second polypeptide of the first polypeptide complex or the first and/or second polypeptide of the second polypeptide complex further comprise a payload moiety.

(14) A method for producing a polypeptide complex, comprising:
incubating:
   (i) a first polypeptide complex, comprising a first polypeptide comprising a CH3 domain and a second polypeptide comprising a CH3 domain;
   wherein the first polypeptide complex is formed by interaction between the first polypeptide and the second polypeptide, the interaction comprising association between the CH3 domain of the first polypeptide and the CH3 domain of the second polypeptide;
   wherein the CH3 domain of the first polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:46; and
   wherein the CH3 domain of the second polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:69; and
   (ii) a second polypeptide complex, comprising a third polypeptide comprising a CH3 domain and a fourth polypeptide comprising a CH3 domain;
   wherein the second polypeptide complex is formed by interaction between the third polypeptide and the fourth polypeptide, the interaction comprising association between the CH3 domain of the third polypeptide and the CH3 domain of the fourth polypeptide;
   wherein the CH3 domain of the third polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:68; and
   wherein the CH3 domain of the fourth polypeptide has at least 70% amino acid sequence identity (e.g. one of ≥75%, ≥80%, ≥85%, ≥90%, ≥91%, ≥92%, ≥93%, ≥94%, ≥95%, ≥96%, ≥97%, ≥98%, ≥99% or 100% amino acid sequence identity) to SEQ ID NO:47;
to form a third polypeptide complex comprising the first polypeptide and the fourth polypeptide (i.e. a polypeptide complex according to (64) above), and/or a fourth polypeptide complex comprising the second polypeptide and the third polypeptide; and
recovering the third polypeptide complex and/or the fourth polypeptide complex;
wherein the first and/or second polypeptide of the first polypeptide complex or the first and/or second polypeptide of the second polypeptide complex further comprise a payload moiety.

### Properties of the Final, Payload-Bearing Polypeptide Complexes

The methods for producing final, payload-bearing polypeptide complexes of the present disclosure, and the final, payload-bearing polypeptide complexes produced by the methods, are provided with advantageous properties.

The methods of the present disclosure provide for the production of payload-bearing polypeptide complexes with exquisite control over the amount of the payload moiety in the final molecule.

For example, where a payload moiety is attached to polypeptides of a donor precursor complex via non-specific conjugation (e.g. of an NHS-bearing payload moiety to lysine residues), the methods of the disclosure provide for the production of final, payload-bearing polypeptide complexes having high consistency in the number of payload moieties, compared to traditional methods for labelling polypeptides and complexes thereof with payload moieties. By way of illustration, Example 2.2 of the present disclosure explains that the methods of the present disclosure can be employed to produce different antibodies (i.e. comprising non-identical antigen-binding domains), from a single pool of ‘donor’ precursor complexes, having a highly similar payload moiety-to-antigen-binding moiety ratio (e.g. drug-antibody ratio; DAR).

As the exact number of payload moieties per molecule can be determined, the final, payload-bearing polypeptide complexes of the present disclosure are particularly well-suited to applications involving relative and/or absolute quantification.

For the same reason, the final, payload-bearing polypeptide complexes of the present disclosure are also particularly well-suited to be employed for the comparison of different functional domains. By way of illustration, as shown in Example 2.2, as molecules comprising different antigen-binding moieties comprise the same amount of payload moiety, their functional properties (in this example, internalisation efficiency) can be directly compared.

The methods of the present disclosure also provide for control over where on the molecule the payload moiety is provided, in molecules labelled by non-specific conjugation. By way of illustration, as shown in Example 2.2, because the non-specific conjugation step is performed independently of the functional moiety (in this example, the antigen-binding domain of the final molecule), the functional moiety is not altered.

It will also be appreciated that the methods of the present disclosure provide for the efficient production of an extraordinarily wide variety of different species, comprising different functional moieties and different payload moieties. Through this modular approach, a ‘donor’ polypeptide complex can donate a polypeptide comprising essentially any payload moiety to a polypeptide of an ‘acceptor’ polypeptide complex, comprising essentially any functional moiety.

### Nucleic Acids and Vectors

The present disclosure provides a nucleic acid, or a plurality of nucleic acids, encoding an polypeptide or polypeptides of a (precursor) polypeptide complex according to the present disclosure. In some embodiments, the nucleic acid(s) comprise or consist of DNA and/or RNA.

The present disclosure also provides a vector, or plurality of vectors, comprising the nucleic acid or plurality of nucleic acids according to the present disclosure.

Nucleic acids and vectors according to the present disclosure may be provided in purified or isolated form, i.e. from other nucleic acid, or naturally-occurring biological material.

The nucleotide sequence may be contained in a vector, e.g. an expression vector. A ‘vector’ as used herein is a nucleic acid molecule used as a vehicle to transfer exogenous nucleic acid into a cell. The vector may be a vector for expression of the nucleic acid in the cell. Such vectors may include a promoter sequence operably linked to the nucleotide sequence encoding the sequence to be expressed. A vector may also include a termination codon and expression enhancers. Any suitable vectors, promoters, enhancers and termination codons known in the art may be used to express a peptide or polypeptide from a vector according to the present disclosure.

The term ‘operably linked’ may include the situation where a selected nucleic acid sequence and regulatory nucleic acid sequence (e.g. promoter and/or enhancer) are covalently linked in such a way as to place the expression of nucleic acid sequence under the influence or control of the regulatory sequence (thereby forming an expression cassette). Thus a regulatory sequence is operably linked to the selected nucleic acid sequence if the regulatory sequence is capable of effecting transcription of the nucleic acid sequence. The resulting transcript(s) may then be translated into a desired peptide(s)/polypeptide(s).

Suitable vectors include plasmids, binary vectors, DNA vectors, mRNA vectors, viral vectors (e.g. gammaretroviral vectors (e.g. murine Leukemia virus (MLV)-derived vectors), lentiviral vectors, adenovirus vectors, adeno-associated virus vectors, vaccinia virus vectors and herpesvirus vectors), transposon-based vectors, and artificial chromosomes (e.g. yeast artificial chromosomes).

In some embodiments, the vector may be a eukaryotic vector, e.g. a vector comprising the elements necessary for expression of protein from the vector in a eukaryotic cell. In some embodiments, the vector may be a mammalian vector, e.g. comprising a cytomegalovirus (CMV) or SV40 promoter to drive protein expression.

Constituent polypeptides of polypeptide complexes according to the present disclosure may be encoded by different nucleic acids of the plurality of nucleic acids, or by different vectors of the plurality of vectors.

### Cells Comprising/Expressing the Polypeptides/Precursor Polypeptide Complexes

The present disclosure also provides a cell comprising or expressing a polypeptide or (precursor) polypeptide complex according to the present disclosure. Also provided is a cell comprising or expressing a nucleic acid or vector according to the present disclosure.

The cell may be a eukaryotic cell, e.g. a mammalian cell. The mammal may be a primate (rhesus, cynomolgous, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate).

In some embodiments, the cell is, or is derived from, a cell type commonly used for the expression of polypeptides, e.g. for use in therapy in humans. Exemplary cells are described e.g. in Kunert and Reinhart, Appl. Microbiol. Biotechnol. (2016) 100:3451-3461 (hereby incorporated by reference in its entirety), and include e.g. CHO, HEK 293, PER.C6, NSO and BHK cells.

The present disclosure also provides a method for producing a cell comprising a nucleic acid or vector according to the present disclosure, comprising introducing a nucleic acid or vector according to the present disclosure into a cell. In some embodiments, introducing an isolated nucleic acid(s) or vector(s) according to the present disclosure into a cell comprises transformation, transfection, electroporation or transduction (e.g. retroviral transduction).

The present disclosure also provides a method for producing a cell expressing/comprising polypeptide or polypeptide complex according to the present disclosure, comprising introducing a nucleic acid or vector according to the present disclosure into a cell. In some embodiments, the methods additionally comprise culturing the cell under conditions suitable for expression of the nucleic acid/vector by the cell. In some embodiments, the methods are performed in vitro.

The present disclosure also provides cells obtained or obtainable by the methods according to the present disclosure.

### Producing the Polypeptides and Precursor Polypeptide Complexes

Polypeptides and (precursor) polypeptide complexes according to the present disclosure may be prepared according to methods for the production of polypeptides known to the skilled person.

Polypeptides may be prepared by chemical synthesis, e.g. liquid or solid phase synthesis. For example, peptides/polypeptides can by synthesised using the methods described in, for example, Chandrudu et al., Molecules (2013), 18:4373-4388, which is hereby incorporated by reference in its entirety.

Alternatively, polypeptides and polypeptide complexes may be produced by recombinant expression. Molecular biology techniques suitable for recombinant production of polypeptides are well known in the art, such as those set out in Green and Sambrook, Molecular Cloning: A Laboratory Manual (4th Edition), Cold Spring Harbor Press (2012), and in Nat Methods. (2008); 5 (2): 135-146 both of which are hereby incorporated by reference in their entirety.

For recombinant production according to the present disclosure, any cell suitable for the expression of polypeptides may be used. The cell may be a prokaryote or eukaryote. In some embodiments the cell is a prokaryotic cell, such as a cell of archaea or bacteria. In some embodiments the bacteria may be Gram-negative bacteria such as bacteria of the family Enterobacteriaceae, for example Escherichia coli. In some embodiments, the cell is a eukaryotic cell such as a yeast cell, a plant cell, insect cell or a mammalian cell, e.g. a cell described hereinabove.

In some cases, the cell is not a prokaryotic cell because some prokaryotic cells do not allow for the same folding or post-translational modifications as eukaryotic cells. In addition, very high expression levels are possible in eukaryotes and proteins can be easier to purify from eukaryotes using appropriate tags. Specific plasmids may also be utilised which enhance secretion of the protein into the media.

In some embodiments polypeptides may be prepared by cell-free-protein synthesis (CFPS), e.g. according to a system described in Zemella et al. Chembiochem (2015) 16 (17): 2420-2431, which is hereby incorporated by reference in its entirety.

Production may involve culture or fermentation of a eukaryotic cell modified to express the polypeptide(s) of interest. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted polypeptide(s). Culture, fermentation and separation techniques are well known to those of skill in the art, and are described, for example, in Green and Sambrook, Molecular Cloning: A Laboratory Manual (4th Edition; incorporated by reference herein above).

Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culturing the cells that express the polypeptide(s), the polypeptide(s) may be isolated or purified (e.g. from cell culture supernatant). Any suitable method for isolating/purifying polypeptides of interest produced by expression from cells in culture may be employed.

In order to isolate expressed polypeptide(s), it may be necessary to separate the cells from nutrient medium. If the polypeptide(s) of interest is secreted from the cells, the cells may be separated by centrifugation from the culture media that contains the secreted polypeptide of interest. If the polypeptide(s) of interest collects within the cell, protein isolation may comprise centrifugation to separate cells from cell culture medium, treatment of the cell pellet with a lysis buffer, and cell disruption e.g. by sonification, rapid freeze-thaw or osmotic lysis.

It may then be desirable to isolate the polypeptide(s) of interest from the supernatant or culture medium, which may contain other protein and non-protein components. A common approach to separating protein components from a supernatant or culture medium is by precipitation. Proteins of different solubilities are precipitated at different concentrations of precipitating agent such as ammonium sulfate. For example, at low concentrations of precipitating agent, water soluble proteins are extracted. Thus, by adding different increasing concentrations of precipitating agent, proteins of different solubilities may be distinguished. Dialysis may be subsequently used to remove ammonium sulfate from the separated proteins.

Other methods for distinguishing different proteins are known in the art, for example ion exchange chromatography and size chromatography. These may be used as an alternative to precipitation or may be performed subsequently to precipitation.

Once the polypeptide(s) of interest has been isolated from culture it may be desired or necessary to concentrate the polypeptide. A number of methods for concentrating proteins are known in the art, such as ultrafiltration or lyophilisation.

### Compositions

The present disclosure also provides compositions comprising the polypeptides, polypeptide complexes, nucleic acids, expression vectors and cells described herein.

The polypeptides, polypeptide complexes, nucleic acids, expression vectors and cells described herein may be formulated as pharmaceutical compositions or medicaments for use in therapeutic and/or prophylactic methods and may comprise a pharmaceutically-acceptable carrier, diluent, excipient or adjuvant. The polypeptides, polypeptide complexes, nucleic acids, expression vectors and cells described herein may be formulated for use in diagnostic and/or prognostic applications.

The compositions of the present disclosure may comprise one or more pharmaceutically-acceptable carriers (e.g. liposomes, micelles, microspheres, nanoparticles), diluents/excipients (e.g. starch, cellulose, a cellulose derivative, a polyol, dextrose, maltodextrin, magnesium stearate), adjuvants, fillers, buffers, preservatives (e.g. vitamin A, vitamin E, vitamin C, retinyl palmitate, selenium, cysteine, methionine, citric acid, sodium citrate, methyl paraben, propyl paraben), anti-oxidants (e.g. vitamin A, vitamin E, vitamin C, retinyl palmitate, selenium), lubricants (e.g. magnesium stearate, talc, silica, stearic acid, vegetable stearin), binders (e.g. sucrose, lactose, starch, cellulose, gelatin, polyethylene glycol (PEG), polyvinylpyrrolidone (PVP), xylitol, sorbitol, mannitol), stabilisers, solubilisers, surfactants (e.g., wetting agents), masking agents or colouring agents (e.g. titanium oxide).

The term ‘pharmaceutically-acceptable’ as used herein pertains to compounds, ingredients, materials, compositions, dosage forms, etc., which are, within the scope of sound medical judgment, suitable for use in contact with the tissues of the subject in question (e.g. a human subject) without excessive toxicity, irritation, allergic response, or other problem or complication, commensurate with a reasonable benefit/risk ratio. Each carrier, diluent, excipient, adjuvant, filler, buffer, preservative, anti-oxidant, lubricant, binder, stabiliser, solubiliser, surfactant, masking agent, colouring agent, flavouring agent or sweetening agent of a composition according to the present disclosure must also be ‘acceptable’ in the sense of being compatible with the other ingredients of the formulation. Suitable carriers, diluents, excipients, adjuvants, fillers, buffers, preservatives, anti-oxidants, lubricants, binders, stabilisers, solubilisers, surfactants, masking agents, colouring agents, flavouring agents or sweetening agents can be found in standard pharmaceutical texts, for example, Remington's ‘The Science and Practice of Pharmacy’ (Ed. A. Adejare), 23rd Edition (2020), Academic Press.

Compositions may be formulated for topical, parenteral, systemic, intracavitary, intravenous, intra-arterial, intramuscular, intrathecal, intraocular, intraconjunctival, intratumoral, subcutaneous, intradermal, intrathecal, oral or transdermal routes of administration. In some embodiments, a pharmaceutical composition/medicament may be formulated for administration by injection or infusion, or administration by ingestion.

Suitable formulations may comprise the relevant article in a sterile or isotonic medium. Medicaments and pharmaceutical compositions may be formulated in fluid, including gel, form. Fluid formulations may be formulated for administration by injection or infusion (e.g. via catheter) to a selected region of the human or animal body.

In some embodiments, the composition is formulated for injection or infusion, e.g. into a blood vessel or tissue/organ of interest.

The present disclosure also provides methods for the production of pharmaceutically useful compositions, such methods of production may comprise one or more steps selected from: producing a polypeptide/polypeptide complex, nucleic acid, expression vector or cell described herein; isolating polypeptide/polypeptide complex, nucleic acid, expression vector or cell described herein; and/or mixing polypeptide/polypeptide complex, nucleic acid, expression vector or cell described herein with a pharmaceutically-acceptable carrier, adjuvant, excipient or diluent.

### Applications

The final, payload-bearing polypeptide complexes of the present disclosure (and articles comprising the same) may be employed in any suitable application, selected in accordance with the identity of the payload moiety and/or the functional moiety.

In particular, the use of the polypeptide complexes of the present disclosure in therapeutic, prophylactic, diagnostic and prognostic applications is contemplated.

For example, where a functional moiety is or comprises a target antigen-binding peptide/polypeptide and the payload moiety is a detectable moiety, the final, payload-bearing polypeptide complex may be employed in methods comprising detecting and/or quantifying the relevant target antigen, e.g. diagnostic and/or prognostic methods (e.g. where the target antigen is a disease-associated antigen).

For example, where a functional moiety is or comprises a target-binding peptide/polypeptide and the payload moiety is a drug moiety, the final, payload-bearing polypeptide complex may be employed in methods comprising depleting cells expressing the relevant target antigen (e.g. at the cell surface), e.g. therapeutic and/or prophylactic methods (e.g. where the target antigen is a disease-associated antigen).

### Kits

The present disclosure also provides kits of parts. In some embodiments, a kit may comprise at least one container having a predetermined quantity of a polypeptide, polypeptide complex, nucleic acid, vector, cell or composition described herein.

Kits may include instructions for use, e.g. in the form of an instruction booklet or leaflet. The instructions may include a protocol for performing any one or more of the methods described herein.

In some aspects and embodiments, a kit may comprise materials for producing a final, payload-bearing polypeptide complex according to the present disclosure. In some embodiments, the kit may comprise a predetermined quantity of a ‘donor’ precursor polypeptide complex, and/or predetermined quantity of an ‘acceptor’ precursor polypeptide complex. In some embodiments, the kit may comprise reagents for producing a final, payload-bearing polypeptide complex according to the present disclosure from ‘donor’ and ‘acceptor’ precursor polypeptide complexes. In some embodiments, the kit may comprise instructions for producing a final, payload-bearing polypeptide complex according to the present disclosure from ‘donor’ and ‘acceptor’ precursor polypeptide complexes, e.g. in accordance with the methods described herein.

Sequences

| | DESCRIPTION | SEQUENCE |
|---|---|---|
| 1 | Human IgG1 CH3 | GQPREPQVYTLPPSRDELTKNQVSLTCLVKGFYPSDIAVEWESNGQPENNYKTTPPVLDS |
| | domain (G1m1) | DGSFFLYSKLTVDKSRWQQGNVFSCSVMHEALHNHYTQKSLSLSPGK |
| 2 | Human IgG1 CH3 | GQPREPQVYTLPPSREEMTKNQVSLTCLVKGFYPSDIAVEWESNGQPENNYKTTPPVLDS |
| | domain (G1m3) | DGSFFLYSKLTVDKSRWQQGNVFSCSVMHEALHNHYTQKSLSLSPGK |
| 3 | Human IgG2 CH3 | GQPREPQVYTLPPSREEMTKNQVSLTCLVKGFYPSDISVEWESNGQPENNYKTTPPMLDS |
| | domain | DGSFFLYSKLTVDKSRWQQGNVFSCSVMHEALHNHYTQKSLSLSPGK |
| 4 | Human IgG3 CH3 | GQPREPQVYTLPPSREEMTKNQVSLTCLVKGFYPSDIAVEWESSGQPENNYNTTPPMLDS |
| | domain | DGSFFLYSKLTVDKSRWQQGNIFSCSVMHEALHNRFTQKSLSLSPGK |
| 5 | Human IgG4 CH3 | GQPREPQVYTLPPSQEEMTKNQVSLTCLVKGFYPSDIAVEWESNGQPENNYKTTPPVLDS |
| | domain | DGSFFLYSRLTVDKSRWQEGNVFSCSVMHEALHNHYTQKSLSLSLGK |
| 6 | Human IgA1 Ig- | PEVHLLPPPSEELALNELVTLTCLARGFSPKDVLVRWLQGSQELPREKYLTWASRQEPSQ |
| | like domain 3 | GTTTFAVTSILRVAAEDWKKGDTFSCMVGHEALPLAFTQKTID |
| 7 | Human IgA2 Ig- | PEVHLLPPPSEELALNELVTLTCLARGFSPKDVLVRWLQGSQELPREKYLTWASRQEPSQ |
| | like domain 3 | GTTTYAVTSILRVAAEDWKKGETFSCMVGHEALPLAFTQKTID |
| 8 | Human IgD Ig- | PAAQAPVKLSLNLLASSDPPEAASWLLCEVSGFSPPNILLMWLEDQREVNTSGFAPARPPP |
| | like domain 3 | QPRSTTFWAWSVLRVPAPPSPQPATYTCVVSHEDSRTLLNASRSLE |
| 9 | Human IgE Ig- | PRGVSAYLSRPSPFDLFIRKSPTITCLVVDLAPSKGTVNLTWSRASGKPVNHSTRKEEKQR |
| | like domain 3 | NGTLTVTSTLPVGTRDWIEGETYQCRVTHPHLPRALMRSTTKTS |
| 10 | Human IgM CH3 | QDTAIRVFAIPPSFASIFLTKSTKLTCLVTDLTTYDSVTISWTRQNGEAVKTHTNISESHPNA |
| | domain | TFSAVGEASICEDDWNSGERFTCTVTHTDLPSPLKQTISRPKG |
| 11 | Human IgG1 CH2 | APELLGGPSVFLFPPKPKDTLMISRTPEVTCVVVDVSHEDPEVKFNWYVDGVEVHNAKT |
| | domain | KPREEQYNSTYRVVSVLTVLHQDWLNGKEYKCKVSNKALPAPIEKTISKAK |
| 12 | Human IgG2 CH2 | APPVAGPSVFLFPPKPKDTLMISRTPEVTCVVVDVSHEDPEVQFNWYVDGVEVHNAKTK |
| | domain | PREEQFNSTFRVVSVLTVVHQDWLNGKEYKCKVSNKGLPAPIEKTISKTK |
| 13 | Human IgG3 CH2 | APELLGGPSVFLFPPKPKDTLMISRTPEVTCVVVDVSHEDPEVQFKWYVDGVEVHNAKT |
| | domain | KPREEQYNSTFRVVSVLTVLHQDWLNGKEYKCKVSNKALPAPIEKTISKTK |
| 14 | Human IgG4 CH2 | APEFLGGPSVFLFPPKPKDTLMISRTPEVTCVVVDVSQEDPEVQFNWYVDGVEVHNAKTK |
| | domain | PREEQFNSTYRVVSVLTVLHQDWLNGKEYKCKVSNKGLPSSIEKTISKAK |
| 15 | Human IgA1 Ig- | PRLSLHRPALEDLLLGSEANLTCTLTGLRDASGVTFTWTPSSGKSAVQGPPERDLCGCYS |
| | like domain 2 | VSSVLPGCAEPWNHGKTFTCTAAYPESKTPLTATLS |
| 16 | Human IgA2 Ig- | PRLSLHRPALEDLLLGSEANLTCTLTGLRDASGATFTWTPSSGKSAVQGPPERDLCGCYS |
| | like domain 2 | VSSVLPGCAQPWNHGETFTCTAAHPELKTPLTANIT |
| 17 | Human IgD Ig- | PAVQDLWLRDKATFTCFVVGSDLKDAHLTWEVAGKVPTGGVEEGLLERHSNGSQSQHS |
| | like domain 2 | RLTLPRSLWNAGTSVTCTLNHPSLPPQRLMA |
| | domain | |
| 18 | Human IgE Ig- | PTVKILQSSCDGGGHFPPTIQLLCLVSGYTPGTINITWLEDGQVMDVDLSTASTTQEGELA |
| | like domain 2 | STQSELTLSQKHWLSDRTYTCQVTYQGHTFEDSTKKCA |
| | domain | |
| 19 | Human IgM CH2 | IAELPPKVSVFVPPRDGFFGNPRKSKLICQATGFSPRQIQVSWLREGKQVGSGVTTDQVQA |
| | domain | EAKESGPTTYKVTSTLTIKESDWLGQSMFTCRVDHRGLTFQQNASSMCVPD |
| 20 | Human IgG1 | EPKSCDKTHTCPPCP |
| | hinge region | |
| 21 | Human IgG2 | ERKCCVECPPCP |
| | hinge region | |
| 22 | Human IgG3 | ELKTPLGDTTHTCPRCPEPKSCDTPPPCPRCPEPKSCDTPPPCPRCPEPKSCDTPPPCPRCP |
| | hinge region | |
| 23 | Human IgG4 | ESKYGPPCPSCP |
| | hinge region | |
| 24 | Human IgG1-4 | GQPREPQVYTLPPSX₁X₂EX₃TKNQVSLWCLVEGFYPSDIX₄VEWESX₅GQPENNY |
| | CH3 domain | X₆TTPPX₇LDSDGSFFLYSX₈LTVDKSRWQX₉GNX₁₀FSCSVMHEALHNX₁₁X₁₂TQKSLSLSX₁₃ |
| | CON + 366W, | GK |
| | 370E | wherein X₁ = R or Q, X₂ = E or D, X₃ = M or L, X₄ = A or S, X₅ = N or |
| | | S, X₆ = K or N, X₇ = V or M, X₈ = K or R, X₉ = Q or E, X₁₀ = V or I, |
| | | X₁₁ = H or R, X₁₂ = Y or F, X₁₃ = P or L |
| 25 | Human IgG1-4 | GQPREPQVYTLPPSX₁X₂KX₃TKNQVSLSCAVKGFYPSDIX₄VEWESX₅GQPENNY |
| | CH3 domain | X₆TTPPX₇LDSDGSFFLVSX₈LTVDKSRWQX₉GNX₁₀FSCSVMHEALHNX₁₁X₁₂TQKSLSLSX₁₃ |
| | CON + 407V, | GK |
| | 366S, 368A, | wherein X₁ = R or Q, X₂ = E or D, X₃ = M or L, X₄ = A or S, X₅ = N or |
| | 357K | S, X₆ = K or N, X₇ = V or M, X₈ = K or R, X₉ = Q or E, X₁₀ = V or I, |
| | | X₁₁ = H or R, X₁₂ = Y or F, X₁₃ = P or L |
| 26 | Human IgG1-4 | GQPREPQVYTLPPCX₁X₂EX₃TKNQVSLWCLVEGFYPSDIX₄VEWESX₅GQPENNY |
| | CH3 domain | X₆TTPPX₇LDSDGSFFLYSX₈LTVDKSRWQX₉GNX₁₀FSCSVMHEALHNX₁₁X₁₂TQKSLSLSX₁₃ |
| | CON + 366W, | GK |
| | 354C, 370E | wherein X₁ = R or Q, X₂ = E or D, X₃ = M or L, X₄ = A or S, X₅ = N or |
| | | S, X₆ = K or N, X₇ = V or M, X₈ = K or R, X₉ = Q or E, X₁₀ = V or I, |
| | | X₁₁ = H or R, X₁₂ = Y or F, X₁₃ = P or L |
| 27 | Human IgG1-4 | GQPREPQVCTLPPSX₁X₂KX₃TKNQVSLSCAVKGFYPSDIX₄VEWESX₅GQPENNY |
| | CH3 domain | X₆TTPPX₇LDSDGSFFLVSX₈LTVDKSRWQX₉GNX₁₀FSCSVMHEALHNX₁₁X₁₂TQKSLSLSX₁₃ |
| | CON + 407V, | GK |
| | 366S, 368A, | wherein X₁ = R or Q, X₂ = E or D, X₃ = M or L, X₄ = A or S, X₅ = N or |
| | 349C, 357K | S, X₆ = K or N, X₇ = V or M, X₈ = K or R, X₉ = Q or E, X₁₀ = V or I, |
| | | X₁₁ = H or R, X₁₂ = Y or F, X₁₃ = P or L |
| 28 | Human IgG1 CH3 | GQPREPQVYTLPPSRDELTKNQVSLWCLVEGFYPSDIAVEWESNGQPENNYKTTPPVLDS |
| | domain (G1m1) + | DGSFFLYSKLTVDKSRWQQGNVFSCSVMHEALHNHYTQKSLSLSPGK |
| | 366W, 370E | |
| 29 | Human IgG1 CH3 | GQPREPQVYTLPPSRDKLTKNQVSLSCAVKGFYPSDIAVEWESNGQPENNYKTTPPVLDS |
| | domain (G1m1) + | DGSFFLVSKLTVDKSRWQQGNVFSCSVMHEALHNHYTQKSLSLSPGK |
| | 407V, 366S, | |
| | 368A, 357K | |
| 30 | Human IgG1 CH3 | GQPREPQVYTLPPCRDELTKNQVSLWCLVEGFYPSDIAVEWESNGQPENNYKTTPPVLDS |
| | domain (G1m1) + | DGSFFLYSKLTVDKSRWQQGNVFSCSVMHEALHNHYTQKSLSLSPGK |
| | 366W, 354C, | |
| | 370E | |
| 31 | Human IgG1 CH3 | GQPREPQVCTLPPSRDKLTKNQVSLSCAVKGFYPSDIAVEWESNGQPENNYKTTPPVLDS |
| | domain (G1m1) + | DGSFFLVSKLTVDKSRWQQGNVFSCSVMHEALHNHYTQKSLSLSPGK |
| | 407V, 366S, | |
| | 368A, 349C, | |
| | 357K | |
| 32 | Human IgG1 CH3 | GQPREPQVYTLPPSREEMTKNQVSLWCLVEGFYPSDIAVEWESNGQPENNYKTTPPVLDS |
| | domain (G1m3) + | DGSFFLYSKLTVDKSRWQQGNVFSCSVMHEALHNHYTQKSLSLSPGK |
| | 366W, 370E | |
| 33 | Human IgG1 CH3 | GQPREPQVYTLPPSREKMTKNQVSLSCAVKGFYPSDIAVEWESNGQPENNYKTTPPVLDS |
| | domain (G1m3) + | DGSFFLVSKLTVDKSRWQQGNVFSCSVMHEALHNHYTQKSLSLSPGK |
| | 407V, 366S, | |
| | 368A, 357K | |
| 34 | Human IgG1 CH3 | GQPREPQVYTLPPCREEMTKNQVSLWCLVEGFYPSDIAVEWESNGQPENNYKTTPPVLDS |
| | domain (G1m3) + | DGSFFLYSKLTVDKSRWQQGNVFSCSVMHEALHNHYTQKSLSLSPGK |
| | 366W, 354C, | |
| | 370E | |
| 35 | Human IgG1 CH3 | GQPREPQVCTLPPSREKMTKNQVSLSCAVKGFYPSDIAVEWESNGQPENNYKTTPPVLDS |
| | domain (G1m3) + | DGSFFLVSKLTVDKSRWQQGNVFSCSVMHEALHNHYTQKSLSLSPGK |
| | 407V, 366S, | |
| | 368A, 349C, | |
| | 357K | |
| 36 | Human IgG2 CH3 | GQPREPQVYTLPPSREEMTKNQVSLWCLVEGFYPSDISVEWESNGQPENNYKTTPPMLDS |
| | domain + 366W, | DGSFFLYSKLTVDKSRWQQGNVFSCSVMHEALHNHYTQKSLSLSPGK |
| | 370E | |
| 37 | Human IgG2 CH3 | GQPREPQVYTLPPSREKMTKNQVSLSCAVKGFYPSDISVEWESNGQPENNYKTTPPMLDS |
| | domain + 407V, | DGSFFLVSKLTVDKSRWQQGNVFSCSVMHEALHNHYTQKSLSLSPGK |
| | 366S, 368A, | |
| | 357K | |
| 38 | Human IgG2 CH3 | GQPREPQVYTLPPCREEMTKNQVSLWCLVEGFYPSDISVEWESNGQPENNYKTTPPMLDS |
| | domain + 366W, | DGSFFLYSKLTVDKSRWQQGNVFSCSVMHEALHNHYTQKSLSLSPGK |
| | 354C, 370E | |
| 39 | Human IgG2 CH3 | GQPREPQVCTLPPSREKMTKNQVSLSCAVKGFYPSDISVEWESNGQPENNYKTTPPMLDS |
| | domain + 407V, | DGSFFLVSKLTVDKSRWQQGNVFSCSVMHEALHNHYTQKSLSLSPGK |
| | 366S, 368A, | |
| | 349C, 357K | |
| 40 | Human IgG3 CH3 | GQPREPQVYTLPPSREEMTKNQVSLWCLVEGFYPSDIAVEWESSGQPENNYNTTPPMLDS |
| | domain + 366W, | DGSFFLYSKLTVDKSRWQQGNIFSCSVMHEALHNRFTQKSLSLSPGK |
| | 370E | |
| 41 | Human IgG3 CH3 | GQPREPQVYTLPPSREKMTKNQVSLSCAVKGFYPSDIAVEWESSGQPENNYNTTPPMLDS |
| | domain + 407V, | DGSFFLVSKLTVDKSRWQQGNIFSCSVMHEALHNRFTQKSLSLSPGK |
| | 366S, 368A, | |
| | 357K | |
| 42 | Human IgG3 CH3 | GQPREPQVYTLPPCREEMTKNQVSLWCLVEGFYPSDIAVEWESSGQPENNYNTTPPMLDS |
| | domain + 366W, | DGSFFLYSKLTVDKSRWQQGNIFSCSVMHEALHNRFTQKSLSLSPGK |
| | 354C, 370E | |
| 43 | Human IgG3 CH3 | GQPREPQVCTLPPSREKMTKNQVSLSCAVKGFYPSDIAVEWESSGQPENNYNTTPPMLDS |
| | domain + 407V, | DGSFFLVSKLTVDKSRWQQGNIFSCSVMHEALHNRFTQKSLSLSPGK |
| | 366S, 368A, | |
| | 349C, 357K | |
| 44 | Human IgG4 CH3 | GQPREPQVYTLPPSQEEMTKNQVSLWCLVEGFYPSDIAVEWESNGQPENNYKTTPPVLDS |
| | domain + 366W, | DGSFFLYSRLTVDKSRWQEGNVFSCSVMHEALHNHYTQKSLSLSLGK |
| | 370E | |
| 45 | Human IgG4 CH3 | GQPREPQVYTLPPSQEKMTKNQVSLSCAVKGFYPSDIAVEWESNGQPENNYKTTPPVLDS |
| | domain + 407V, | DGSFFLVSRLTVDKSRWQEGNVFSCSVMHEALHNHYTQKSLSLSLGK |
| | 366S, 368A, | |
| | 357K | |
| 46 | Human IgG4 CH3 | GQPREPQVYTLPPCQEEMTKNQVSLWCLVEGFYPSDIAVEWESNGQPENNYKTTPPVLDS |
| | domain + 366W, | DGSFFLYSRLTVDKSRWQEGNVFSCSVMHEALHNHYTQKSLSLSLGK |
| | 354C, 370E | |
| 47 | Human IgG4 CH3 | GQPREPQVCTLPPSQEKMTKNQVSLSCAVKGFYPSDIAVEWESNGQPENNYKTTPPVLDS |
| | domain + 407V, | DGSFFLVSRLTVDKSRWQEGNVFSCSVMHEALHNHYTQKSLSLSLGK |
| | 366S, 368A, | |
| | 349C, 357K | |
| 48 | Human IgG1-4 | GQPREPQVYTLPPSX₁X₂EX₃TKNQVSLWCLVKGFYPSDIX₄VEWESX₅GQPENNY |
| | CH3 domain | X₆TTPPX₇LDSDGSFFLYSX₈LTVDKSRWQX₉GNX₁₀FSCSVMHEALHNX₁₁X₁₂TQKSLSLSX₁₃ |
| | | GK |
| | CON + 366W | wherein X₁ = R or Q, X₂ = E or D, X₃ = M or L, X₄ = A or S, X₅ = N or |
| | | S, X₆ = K or N, X₇ = V or M, _{X8} = K or R, X₉ = Q or E, X₁₀ = V or I, |
| | | X₁₁ = H or R, X₁₂ = Y or F, X₁₃ = P or L |
| 49 | Human IgG1-4 | GQPREPQVYTLPPSX₁X₂EX₃TKNQVSLSCAVKGFYPSDIX₄VEWESX₅GQPENNY |
| | CH3 domain | X₆TTPPX₇LDSDGSFFLVSX₈LTVDKSRWQX₉GNX₁₀FSCSVMHEALHNX₁₁X₁₂TQKSLSLSX₁₃ |
| | CON + 407V, | GK |
| | 366S, 368A | wherein X₁ = R or Q, X₂ = E or D, X₃ = M or L, X₄ = A or S, X₅ = N or |
| | | S, X₆ = K or N, X₇ = V or M, X₈ = K or R, X₉ = Q or E, X₁₀ = V or I, |
| | | X₁₁ = H or R, X₁₂ = Y or F, X₁₃ = P or L |
| 50 | Human IgG1-4 | GQPREPQVYTLPPCX₁X₂EX₃TKNQVSLWCLVKGFYPSDIX₄VEWESX₅GQPENNY |
| | CH3 domain | X₆TTPPX₇LDSDGSFFLYSX₈LTVDKSRWQX₉GNX₁₀FSCSVMHEALHNX₁₁X₁₂TQKSLSLSX₁₃ |
| | CON + 366W, | GK |
| | 354C | wherein X₁ = R or Q, X₂ = E or D, X₃ = M or L, X₄ = A or S, X₅ = N or |
| | | S, X₆ = K or N, X₇ = V or M, X₈ = K or R, X₉ = Q or E, X₁₀ = V or I, |
| | | X₁₁ = H or R, X₁₂ = Y or F, X₁₃ = P or L |
| 51 | Human IgG1-4 | GQPREPQVCTLPPSX₁X₂EX₃TKNQVSLSCAVKGFYPSDIX₄VEWESX₅GQPENNY |
| | CH3 domain | X₆TTPPX₇LDSDGSFFLVSX₈LTVDKSRWQX₉GNX₁₀FSCSVMHEALHNX₁₁X₁₂TQKSLSLSX₁₃ |
| | CON + 407V, | GK |
| | 366S, 368A, | wherein X₁ = R or Q, X₂ = E or D, X₃ = M or L, X₄ = A or S, X₅ = N or |
| | 349C | S, X₆ = K or N, X₇ = V or M, X₈ = K or R, X₉ = Q or E, X₁₀ = V or I, |
| | | X₁₁ = H or R, X₁₂ = Y or F, X₁₃ = P or L |
| 52 | Human IgG1 | GQPREPQVYTLPPSRDELTKNQVSLWCLVKGFYPSDIAVEWESNGQPENNYKTTPPVLDS |
| | (G1m1) CH3 | DGSFFLYSKLTVDKSRWQQGNVFSCSVMHEALHNHYTQKSLSLSPGK |
| | domain + 366W | |
| 53 | Human IgG1 | GQPREPQVYTLPPSRDELTKNQVSLSCAVKGFYPSDIAVEWESNGQPENNYKTTPPVLDS |
| | (G1m1) CH3 | DGSFFLVSKLTVDKSRWQQGNVFSCSVMHEALHNHYTQKSLSLSPGK |
| | domain + 407V, | |
| | 366S, 368A | |
| 54 | Human IgG1 | GQPREPQVYTLPPCRDELTKNQVSLWCLVKGFYPSDIAVEWESNGQPENNYKTTPPVLDS |
| | (G1m1) CH3 | DGSFFLYSKLTVDKSRWQQGNVFSCSVMHEALHNHYTQKSLSLSPGK |
| | domain + 366W, | |
| | 354C | |
| 55 | Human IgG1 | GQPREPQVCTLPPSRDELTKNQVSLSCAVKGFYPSDIAVEWESNGQPENNYKTTPPVLDS |
| | (G1m1) CH3 | DGSFFLVSKLTVDKSRWQQGNVFSCSVMHEALHNHYTQKSLSLSPGK |
| | domain + 407V, | |
| | 366S, 368A, | |
| | 349C | |
| 56 | Human IgG1 | GQPREPQVYTLPPSREEMTKNQVSLWCLVKGFYPSDIAVEWESNGQPENNYKTTPPVLDS |
| | (G1m3) CH3 | DGSFFLYSKLTVDKSRWQQGNVFSCSVMHEALHNHYTQKSLSLSPGK |
| | domain + 366W | |
| 57 | Human IgG1 | GQPREPQVYTLPPSREEMTKNQVSLSCAVKGFYPSDIAVEWESNGQPENNYKTTPPVLDS |
| | (G1m3) CH3 | DGSFFLVSKLTVDKSRWQQGNVFSCSVMHEALHNHYTQKSLSLSPGK |
| | domain + 407V, | |
| | 366S, 368A | |
| 58 | Human IgG1 | GQPREPQVYTLPPCREEMTKNQVSLWCLVKGFYPSDIAVEWESNGQPENNYKTTPPVLD |
| | (G1m3) CH3 | SDGSFFLYSKLTVDKSRWQQGNVFSCSVMHEALHNHYTQKSLSLSPGK |
| | domain + 366W, | |
| | 354C | |
| 59 | Human IgG1 | GQPREPQVCTLPPSREEMTKNQVSLSCAVKGFYPSDIAVEWESNGQPENNYKTTPPVLDS |
| | (G1m3) CH3 | DGSFFLVSKLTVDKSRWQQGNVFSCSVMHEALHNHYTQKSLSLSPGK |
| | domain + 407V, | |
| | 366S, 368A, | |
| | 349C | |
| 60 | Human IgG2 CH3 | GQPREPQVYTLPPSREEMTKNQVSLWCLVKGFYPSDISVEWESNGQPENNYKTTPPMLDS |
| | domain + 366W | DGSFFLYSKLTVDKSRWQQGNVFSCSVMHEALHNHYTQKSLSLSPGK |
| 61 | Human IgG2 CH3 | GQPREPQVYTLPPSREEMTKNQVSLSCAVKGFYPSDISVEWESNGQPENNYKTTPPMLDS |
| | domain + 407V, | DGSFFLVSKLTVDKSRWQQGNVFSCSVMHEALHNHYTQKSLSLSPGK |
| | 366S, 368A | |
| 62 | Human IgG2 CH3 | GQPREPQVYTLPPCREEMTKNQVSLWCLVKGFYPSDISVEWESNGQPENNYKTTPPMLD |
| | domain + 366W, | SDGSFFLYSKLTVDKSRWQQGNVFSCSVMHEALHNHYTQKSLSLSPGK |
| | 354C | |
| 63 | Human IgG2 CH3 | GQPREPQVCTLPPSREEMTKNQVSLSCAVKGFYPSDISVEWESNGQPENNYKTTPPMLDS |
| | domain + 407V, | DGSFFLVSKLTVDKSRWQQGNVFSCSVMHEALHNHYTQKSLSLSPGK |
| | 366S, 368A, | |
| | 349C | |
| 64 | Human IgG3 CH3 | GQPREPQVYTLPPSREEMTKNQVSLWCLVKGFYPSDIAVEWESSGQPENNYNTTPPMLDS |
| | domain + 366W | DGSFFLYSKLTVDKSRWQQGNIFSCSVMHEALHNRFTQKSLSLSPG |
| 65 | Human IgG3 CH3 | GQPREPQVYTLPPSREEMTKNQVSLSCAVKGFYPSDIAVEWESSGQPENNYNTTPPMLDS |
| | domain + 407V, | DGSFFLVSKLTVDKSRWQQGNIFSCSVMHEALHNRFTQKSLSLSPG |
| | 366S, 368A | |
| 66 | Human IgG3 CH3 | GQPREPQVYTLPPCREEMTKNQVSLWCLVKGFYPSDIAVEWESSGQPENNYNTTPPMLD |
| | domain + 366W, | SDGSFFLYSKLTVDKSRWQQGNIFSCSVMHEALHNRFTQKSLSLSPG |
| | 354C | |
| 67 | Human IgG3 CH3 | GQPREPQVCTLPPSREEMTKNQVSLSCAVKGFYPSDIAVEWESSGQPENNYNTTPPMLDS |
| | domain + 407V, | DGSFFLVSKLTVDKSRWQQGNIFSCSVMHEALHNRFTQKSLSLSPG |
| | 366S, 368A, | |
| | 349C | |
| 68 | Human IgG4 CH3 | GQPREPQVYTLPPSQEEMTKNQVSLWCLVKGFYPSDIAVEWESNGQPENNYKTTPPVLDS |
| | domain + 366W | DGSFFLYSRLTVDKSRWQEGNVFSCSVMHEALHNHYTQKSLSLSLGK |
| 69 | Human IgG4 CH3 | GQPREPQVYTLPPSQEEMTKNQVSLSCAVKGFYPSDIAVEWESNGQPENNYKTTPPVLDS |
| | domain + 407V, | DGSFFLVSRLTVDKSRWQEGNVFSCSVMHEALHNHYTQKSLSLSLGK |
| | 366S, 368A | |
| 70 | Human IgG4 CH3 | GQPREPQVYTLPPCQEEMTKNQVSLWCLVKGFYPSDIAVEWESNGQPENNYKTTPPVLD |
| | domain + 366W, | SDGSFFLYSRLTVDKSRWQEGNVFSCSVMHEALHNHYTQKSLSLSLGK |
| | 354C | |
| 71 | Human IgG4 CH3 | GQPREPQVCTLPPSQEEMTKNQVSLSCAVKGFYPSDIAVEWESNGQPENNYKTTPPVLDS |
| | domain + 407V, | DGSFFLVSRLTVDKSRWQEGNVFSCSVMHEALHNHYTQKSLSLSLGK |
| | 366S, 368A, | |
| | 349C | |
| 72 | 4D5-8 LC | DIQMTQSPSSLSASVGDRVTITCRASQDVNTAVAWYQQKPGKAPKLLIYSASFLYSGVPS |
| | | RFSGSRSGTDFTLTISSLQPEDFATYYCQQHYTTPPTFGQGTKVEIKRTVAAPSVFIFPPSDE |
| | | QLKSGTASVVCLLNNFYPREAKVQWKVDNALQSGNSQESVTEQDSKDSTYSLSSTLTLS |
| | | KADYEKHKVYACEVTHQGLSSPVTKSFNRGEC |
| 73 | 4D5-8 IgG1 HC | EVQLVESGGGLVQPGGSLRLSCAASGFNIKDTYIHWVRQAPGKGLEWVARIYPTNGYTR |
| | (407V, 366S, | YADSVKGRFTISADTSKNTAYLQMNSLRAEDTAVYYCSRWGGDGFYAMDYWGQGTLV |
| | 368A, 349C) | TVSSASTKGPSVFPLAPSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSCDKTHTCPPCPAPELL |
| | | GGPSVFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVCTLPPS |
| | | RDELTKNQVSLSCAVKGFYPSDIAVEWESNGQPENNYKTTPPVLDSDGSFFLVSKLTVDK |
| | | SRWQQGNVFSCSVMHEALHNHYTQKSLSLSPGK |
| 74 | Dummy IgG1 | DKTHTCPPCPAPELLGGPSVFLFPPKPKDTLMISRTPEVTCVVVDVSHEDPEVKFNWYVD |
| | hinge CH2-CH3 | GVEVHNAKTKPREEQYNSTYRVVSVLTVLHQDWLNGKEYKCKVSNKALPAPIEKTISKA |
| | (366W, 370E) + | KGQPREPQVYTLPPSRDELTKNQVSLWCLVEGFYPSDIAVEWESNGQPENNYKTTPPVLD |
| | C-tag | SDGSFFLYSKLTVDKSRWQQGNVFSCSVMHEALHNHYTQKSLSLSPGKGGGGSEPEA |
| 75 | 4D5-8 IgG1 HC | EVQLVESGGGLVQPGGSLRLSCAASGFNIKDTYIHWVRQAPGKGLEWVARIYPTNGYTR |
| | (366W, 354C) | YADSVKGRFTISADTSKNTAYLQMNSLRAEDTAVYYCSRWGGDGFYAMDYWGQGTLV |
| | | TVSSASTKGPSVFPLAPSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSCDKTHTCPPCPAPELL |
| | | GGPSVFLFPPKPKDTLMISRTPEVTCVVVDVSHEDPEVKFNWYVDGVEVHNAKTKPREE |
| | | QYNSTYRVVSVLTVLHQDWLNGKEYKCKVSNKALPAPIEKTISKAKGQPREPQVYTLPPC |
| | | RDELTKNQVSLWCLVKGFYPSDIAVEWESNGQPENNYKTTPPVLDSDGSFFLYSKLTVDK |
| | | SRWQQGNVFSCSVMHEALHNHYTQKSLSLSPGK |
| 76 | Dummy IgG1 | DKTHTCPPCPAPELLGGPSVFLFPPKPKDTLMISRTPEVTCVVVDVSHEDPEVKFNWYVD |
| | hinge CH2-CH3 | GVEVHNAKTKPREEQYNSTYRVVSVLTVLHQDWLNGKEYKCKVSNKALPAPIEKTISKA |
| | (407V, 366S, | KGQPREPQVYTLPPSRDKLTKNQVSLSCAVKGFYPSDIAVEWESNGQPENNYKTTPPVLD |
| | 368A, 357K) + C- | SDGSFFLVSKLTVDKSRWQQGNVFSCSVMHEALHNHYTQKSLSLSPGKGGGGSEPEA |
| | tag | |
| 77 | F5 LC | QSVLTQPPSVSGAPGQRVTISCTGSSSNIGAGYGVHWYQQLPGTAPKLLIYGNTNRPSGVP |
| | | DRFSGFKSGTSASLAITGLQAEDEADYYCQSYDSSLSGWVFGGGTKLTVLRTVAAPSVFIF |
| | | PPSDEQLKSGTASVVCLLNNFYPREAKVQWKVDNALQSGNSQESVTEQDSKDSTYSLSST |
| | | LTLSKADYEKHKVYACEVTHQGLSSPVTKSFNRGEC |
| 78 | F5 IgG1 HC | QVQLVESGGGLVQPGGSLRLSCAASGFTFRSYAMSWVRQAPGKGLEWVSAISGRGDNTY |
| | (407V, 366S, | YADSVKGRFTISRDNSKNTLYLQMNSLRAEDTAVYYCAKMTSNAFAFDYWGQGTLVTV |
| | 368A, 349C) | SSASTKGPSVFPLAPSSKSTSGGTAALGCLVKDYFPEPVTVSWNSGALTSGVHTFPAVLQS |
| | | SGLYSLSSVVTVPSSSLGTQTYICNVNHKPSNTKVDKKVEPKSCDKTHTCPPCPAPELLGG |
| | | PSVFLFPPKPKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREPQVCTLPPSRD |
| | | ELTKNQVSLSCAVKGFYPSDIAVEWESNGQPENNYKTTPPVLDSDGSFFLVSKLTVDKSR |
| | | WQQGNVFSCSVMHEALHNHYTQKSLSLSPGK |
| 79 | F5 scFv Dummy | DKTHTCPPCPAPELLGGPSVFLFPPKPKDTLMISRTPEVTCVVVDVSHEDPEVKFNWYVD |
| | IgG1 hinge CH2- | GVEVHNAKTKPREEQYNSTYRVVSVLTVLHQDWLNGKEYKCKVSNKALPAPIEKTISKA |
| | CH3 (407V, | KGQPREPQVCTLPPSRDELTKNQVSLSCAVKGFYPSDIAVEWESNGQPENNYKTTPPVLD |
| | 366S, 368A, 349C) | SDGSFFLVSKLTVDKSRWQQGNVFSCSVMHEALHNHYTQKSLSLSPGKGGGGSGGGGSQ |
| | | SVLTQPPSVSGAPGQRVTISCTGSSSNIGAGYGVHWYQQLPGTAPKLLIYGNTNRPSGVPD |
| | | RFSGFKSGTSASLAITGLQAEDEADYYCQSYDSSLSGWVFGGGTKLTVLGGGGSGGGGS |
| | | GGGGSGGGSGQVQLVESGGGLVQPGGSLRLSCAASGFTFRSYAMSWVRQAPGKGLEWV |
| | | SAISGRGDNTYYADSVKGRFTISRDNSKNTLYLQMNSLRAEDTAVYYCAKMTSNAFAFD |
| | | YWGQGTLVTVSS |
| 80 | C6.5 LC | QSVLTQPPSVSAAPGQKVTISCSGSSSNIGNNYVSWYQQLPGTAPKLLIYGHTNRPAGVPD |
| | | RFSGSKSGTSASLAISGFRSEDEADYYCAAWDDSLSGWVFGGGTKLTVLGRTVAAPSVFI |
| | | FPPSDEQLKSGTASVVCLLNNFYPREAKVQWKVDNALQSGNSQESVTEQDSKDSTYSLSS |
| | | TLTLSKADYEKHKVYACEVTHQGLSSPVTKSFNRGEC |
| 81 | C6.5 IgG1 HC | QVQLLQSGAELKKPGESLKISCKGSGYSFTSYWIAWVRQMPGKGLEYMGLIYPGDSDTK |
| | (407V, 366S, | YSPSFQGQVTISVDKSVSTAYLQWSSLKPSDSAVYFCARHDVGYCSSSNCAKWPEYFQH |
| | 368A, 349C) | WGQGTLVTVSSASTKGPSVFPLAPSSKSTSGGTAALGCLVKDYFPEPVTVSWNSGALTSG |
| | | VHTFPAVLQSSGLYSLSSVVTVPSSSLGTQTYICNVNHKPSNTKVDKKVEPKSCDKTHTCP |
| | | PCPAPELLGGPSVFLFPPKPKDTLMISRTPEVTCVVVDVSHEDPEVKFNWYVDGVEVHNA |
| | | KTKPREEQYNSTYRVVSVLTVLHQDWLNGKEYKCKVSNKALPAPIEKTISKAKGQPREP |
| | | QVCTLPPSRDELTKNQVSLSCAVKGFYPSDIAVEWESNGQPENNYKTTPPVLDSDGSFFL |
| | | VSKLTVDKSRWQQGNVFSCSVMHEALHNHYTQKSLSLSPGK |
| 82 | C6.5 scFv | DKTHTCPPCPAPELLGGPSVFLFPPKPKDTLMISRTPEVTCVVVDVSHEDPEVKFNWYVD |
| | Dummy IgG1 | GVEVHNAKTKPREEQYNSTYRVVSVLTVLHQDWLNGKEYKCKVSNKALPAPIEKTISKA |
| | hinge CH2-CH3 | KGQPREPQVCTLPPSRDELTKNQVSLSCAVKGFYPSDIAVEWESNGQPENNYKTTPPVLD |
| | (407V, 366S, | SDGSFFLVSKLTVDKSRWQQGNVFSCSVMHEALHNHYTQKSLSLSPGKGGGGSGGGGSQ |
| | 368A, 349C) | SVLTQPPSVSAAPGQKVTISCSGSSSNIGNNYVSWYQQLPGTAPKLLIYGHTNRPAGVPDR |
| | | FSGSKSGTSASLAISGFRSEDEADYYCAAWDDSLSGWVFGGGTKLTVLGGGGGSGGGGS |
| | | GGGGSGGGSGQVQLLQSGAELKKPGESLKISCKGSGYSFTSYWIAWVRQMPGKGLEYM |
| | | GLIYPGDSDTKYSPSFQGQVTISVDKSVSTAYLQWSSLKPSDSAVYFCARHDVGYCSSSN |
| | | CAKWPEYFQHWGQGTLVTVSS |
| 83 | C225 LC | DILLTQSPVILSVSPGERVSFSCRASQSIGTNIHWYQQRTNGSPRLLIKYASESISGIPSRFSG |
| | | SGSGTDFTLSINSVESEDIADYYCQQNNNWPTTFGAGTKLELKRTVAAPSVFIFPPSDEQL |
| | | KSGTASVVCLLNNFYPREAKVQWKVDNALQSGNSQESVTEQDSKDSTYSLSSTLTLSKA |
| | | DYEKHKVYACEVTHQGLSSPVTKSFNRGEC |
| 84 | C225 IgG1 HC | QVQLKQSGPGLVQPSQSLSITCTVSGFSLTNYGVHWVRQSPGKGLEWLGVIWSGGNTDY |
| | (407V, 366S, | NTPFTSRLSINKDNSKSQVFFKMNSLQSNDTAIYYCARALTYYDYEFAYWGQGTLVTVSA |
| | 368A, 349C) | ASTKGPSVFPLAPSSKSTSGGTAALGCLVKDYFPEPVTVSWNSGALTSGVHTFPAVLQSSG |
| | | LYSLSSVVTVPSSSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNAKTKPREEQYNS |
| | | TYRVVSVLTVLHQDWLNGKEYKCKVSNKALPAPIEKTISKAKGQPREPQVCTLPPSRDEL |
| | | TKNQVSLSCAVKGFYPSDIAVEWESNGQPENNYKTTPPVLDSDGSFFLVSKLTVDKSRWQ |
| | | QGNVFSCSVMHEALHNHYTQKSLSLSPGK |
| 85 | C225 IgG1 HC | QVQLKQSGPGLVQPSQSLSITCTVSGFSLTNYGVHWVRQSPGKGLEWLGVIWSGGNTDY |
| | (366W, 354C) | NTPFTSRLSINKDNSKSQVFFKMNSLQSNDTAIYYCARALTYYDYEFAYWGQGTLVTVSA |
| | | ASTKGPSVFPLAPSSKSTSGGTAALGCLVKDYFPEPVTVSWNSGALTSGVHTFPAVLQSSG |
| | | LYSLSSVVTVPSSSLGTQTYICNVNHKPSNTKVDKKVEPKSCDKTHTCPPCPAPELLGGPS |
| | | VFLFPPKPKDTLMISRTPEVTCVVVDVSHEDPEVKFNWYVDGVEVHNAKTKPREEQYNS |
| | | TYRVVSVLTVLHQDWLNGKEYKCKVSNKALPAPIEKTISKAKGQPREPQVYTLPPCRDEL |
| | | TKNQVSLWCLVKGFYPSDIAVEWESNGQPENNYKTTPPVLDSDGSFFLYSKLTVDKSRW |
| | | QQGNVFSCSVMHEALHNHYTQKSLSLSPGK |
| 86 | Dummy IgG1 | DKTHTCPPCPAPELLGGPSVFLFPPKPKDTLMISRTPEVTCVVVDVSHEDPEVKFNWYVD |
| | hinge CH2-CH3 | GVEVHNAKTKPREEQYNSTYRVVSVLTVLHQDWLNGKEYKCKVSNKALPAPIEKTISKA |
| | (366W, 354C) | KGQPREPQVYTLPPCRDELTKNQVSLWCLVKGFYPSDIAVEWESNGQPENNYKTTPPVL |
| | | DSDGSFFLYSKLTVDKSRWQQGNVFSCSVMHEALHNHYTQKSLSLSPGK |
| 87 | Dummy IgG1 | DKTHTCPPCPAPELLGGPSVFLFPPKPKDTLMISRTPEVTCVVVDVSHEDPEVKFNWYVD |
| | hinge CH2-CH3 | GVEVHNAKTKPREEQYNSTYRVVSVLTVLHQDWLNGKEYKCKVSNKALPAPIEKTISKA |
| | (407V, 366S, | KGQPREPQVCTLPPSRDELTKNQVSLSCAVKGFYPSDIAVEWESNGQPENNYKTTPPVLD |
| | 368A, 349C) | SDGSFFLVSKLTVDKSRWQQGNVFSCSVMHEALHNHYTQKSLSLSPGK |
| 88 | Dummy IgG1 | DKTHTCPPCPAPEAAGGPSVFLFPPKPKDTLMISRTPEVTCVVVDVSHEDPEVKFNWYVD |
| | hinge CH2-CH3 | GVEVHNAKTKPREEQYNSTYRVVSVLTVLHQDWLNGKEYKCKVSNKALGAPIEKTISKA |
| | (234A, 235A, | KGQPREPQVYTLPPSRDKLTKNQVSLSCAVKGFYPSDIAVEWESNGQPENNYKTTPPVLD |
| | 329G, 407V, | SDGSFFLVSKLTVDKSRWQQGNVFSCSVMHEALHNHYTQKSLSLSPGKGGGGSEPEA |
| | 366S, 368A, | |
| | 349C) + C-tag | |
| 89 | Dummy IgG1 | DKTHTCPPCPAPEAAGGPSVFLFPPKPKDTLMISRTPEVTCVVVDVSHEDPEVKFNWYVD |
| | hinge CH2-CH3 | GVEVHNAKTKPREEQYNSTYRVVSVLTVLHQDWLNGKEYKCKVSNKALGAPIEKTISKA |
| | (234A, 235A, | KGQPREPQVYTLPPCRDELTKNQVSLWCLVKGFYPSDIAVEWESNGQPENNYKTTPPVL |
| | 329G, 366W, | DSDGSFFLYSKLTVDKSRWQQGNVFSCSVMHEALHNHYTQKSLSLSPGK |
| | 354C) | |
| 90 | Dummy IgG1 | DKTHTCPPCPAPEAAGGPSVFLFPPKPKDTLMISRTPEVTCVVVDVSHEDPEVKFNWYVD |
| | hinge CH2-CH3 | GVEVHNAKTKPREEQYNSTYRVVSVLTVLHQDWLNGKEYKCKVSNKALGAPIEKTISKA |
| | (234A, 235A, | KGQPREPQVYTLPPSRDELTKNQVSLWCLVEGFYPSDIAVEWESNGQPENNYKTTPPVLD |
| | 329G, 366W, | SDGSFFLYSKLTVDKSRWQQGNVFSCSVMHEALHNHYTQKSLSLSPGKGGGGSEPEA |
| | 370E) + C-tag | |
| 91 | Dummy IgG1 | DKTHTCPPCPAPEAAGGPSVFLFPPKPKDTLMISRTPEVTCVVVDVSHEDPEVKFNWYVD |
| | hinge CH2-CH3 | GVEVHNAKTKPREEQYNSTYRVVSVLTVLHQDWLNGKEYKCKVSNKALGAPIEKTISKA |
| | (234A, 235A, | KGQPREPQVCTLPPSRDELTKNQVSLSCAVKGFYPSDIAVEWESNGQPENNYKTTPPVLD |
| | 329G, 407V, | SDGSFFLVSKLTVDKSRWQQGNVFSCSVMHEALHNHYTQKSLSLSPGK |
| | 366S, 368A, | |
| | 349C) | |
| 92 | IgG1 hinge CH2- | DKTHTCPPCPAPELLGGPSVFLFPPKPKDTLMISRTPEVTCVVVDVSHEDPEVKFNWYVD |
| | CH3 (366W, | GVEVHNAKTKPREEQYNSTYRVVSVLTVLHQDWLNGKEYKCKVSNKALPAPIEKTISKA |
| | 354C) + mEGFP | KGQPREPQVYTLPPCRDELTKNQVSLWCLVKGFYPSDIAVEWESNGQPENNYKTTPPVL |
| | | DSDGSFFLYSKLTVDKSRWQQGNVFSCSVMHEALHNHYTQKSLSLSPGKGGGGSGGGGS |
| | | VSKGEELFTGVVPILVELDGDVNGHKFSVSGEGEGDATYGKLTLKFICTTGKLPVPWPTL |
| | | VTTLTYGVQCFSRYPDHMKQHDFFKSAMPEGYVQERTIFFKDDGNYKTRAEVKFEGDTL |
| | | VNRIELKGIDFKEDGNILGHKLEYNYNSHNVYIMADKQKNGIKVNFKIRHNIEDGSVQLA |
| | | DHYQQNTPIGDGPVLLPDNHYLSTQSKLSKDPNEKRDHMVLLEFVTAAGITLGMDELYK |
| 93 | IgG1 hinge CH2- | DKTHTCPPCPAPELLGGPSVFLFPPKPKDTLMISRTPEVTCVVVDVSHEDPEVKFNWYVD |
| | CH3 (407V, | GVEVHNAKTKPREEQYNSTYRVVSVLTVLHQDWLNGKEYKCKVSNKALPAPIEKTISKA |
| | 366S, 368A, | KGQPREPQVCTLPPSRDELTKNQVSLSCAVKGFYPSDIAVEWESNGQPENNYKTTPPVLD |
| | 349C) + mEGFP | SDGSFFLVSKLTVDKSRWQQGNVFSCSVMHEALHNHYTQKSLSLSPGKGGGGSGGGGSV |
| | | SKGEELFTGVVPILVELDGDVNGHKFSVSGEGEGDATYGKLTLKFICTTGKLPVPWPTLV |
| | | TTLTYGVQCFSRYPDHMKQHDFFKSAMPEGYVQERTIFFKDDGNYKTRAEVKFEGDTLV |
| | | NRIELKGIDFKEDGNILGHKLEYNYNSHNVYIMADKQKNGIKVNFKIRHNIEDGSVQLAD |
| | | HYQQNTPIGDGPVLLPDNHYLSTQSKLSKDPNEKRDHMVLLEFVTAAGITLGMDELYK |
| 94 | IgG1 hinge CH2- | DKTHTCPPCPAPELLGGPSVFLFPPKPKDTLMISRTPEVTCVVVDVSHEDPEVKFNWYVD |
| | CH3 (366W, | GVEVHNAKTKPREEQYNGGGSYRYRQGGGSSTYRVVSVLTVLHQDWLNGKEYKCKVS |
| | 354C) + Q-tag (at | NKALPAPIEKTISKAKGQPREPQVYTLPPCRDELTKNQVSLWCLVKGFYPSDIAVEWESN |
| | 297) | GQPENNYKTTPPVLDSDGSFFLYSKLTVDKSRWQQGNVFSCSVMHEALHNHYTQKSLSL |
| | | SPGK |
| 95 | IgG1 hinge CH2- | DKTHTCPPCPAPELLGGPSVFLFPPKPKDTLMISRTPEVTCVVVDVSHEDPEVKFNWYVD |
| | CH3 (407V, | GVEVHNAKTKPREEQYNGGGSYRYRQGGGSSTYRVVSVLTVLHQDWLNGKEYKCKVS |
| | 366S, 368A, | NKALPAPIEKTISKAKGQPREPQVCTLPPSRDELTKNQVSLSCAVKGFYPSDIAVEWESNG |
| | 349C) + Q-tag (at | QPENNYKTTPPVLDSDGSFFLVSKLTVDKSRWQQGNVFSCSVMHEALHNHYTQKSLSLS |
| | 297) | PGK |
| 96 | IgG1 hinge CH2- | DKTHTCPPCPAPEAAGGPSVFLFPPKPKDTLMISRTPEVTCVVVDVSHEDPEVKFNWYVD |
| | CH3 (234A, | GVEVHNAKTKPREEQYNGGGSYRYRQGGGSSTYRVVSVLTVLHQDWLNGKEYKCKVS |
| | 235A, 329G, | NKALGAPIEKTISKAKGQPREPQVYTLPPCRDELTKNQVSLWCLVKGFYPSDIAVEWESN |
| | 366W, 354C) + | GQPENNYKTTPPVLDSDGSFFLYSKLTVDKSRWQQGNVFSCSVMHEALHNHYTQKSLSL |
| | Q-tag (at 297) | SPGK |
| 97 | IgG1 hinge CH2- | DKTHTCPPCPAPEAAGGPSVFLFPPKPKDTLMISRTPEVTCVVVDVSHEDPEVKFNWYVD |
| | CH3 (234A, | GVEVHNAKTKPREEQYNGGGSYRYRQGGGSSTYRVVSVLTVLHQDWLNGKEYKCKVS |
| | 235A, 329G, | NKALGAPIEKTISKAKGQPREPQVCTLPPSRDELTKNQVSLSCAVKGFYPSDIAVEWESNG |
| | 407V, 366S, | QPENNYKTTPPVLDSDGSFFLVSKLTVDKSRWQQGNVFSCSVMHEALHNHYTQKSLSLS |
| | 368A, 349C) + Q- | PGK |
| | tag (at 297) | |
| 98 | IgG1 hinge CH2- | DKTHTCPPCPAPELLGGPSVFLFPPKPKDTLMISRTPEVTCVVVDVSHEDPEVKFNWYVD |
| | CH3 (366W, | GVEVHNAKTKPREEQYNSTYRVVSVLTVLHQDWLNGKEYKCKVSNKALPAPIEKTISKA |
| | 354C) + Q-tag (C | KGQPREPQVYTLPPCRDELTKNQVSLWCLVKGFYPSDIAVEWESNGQPENNYKTTPPVL |
| | term) | DSDGSFFLYSKLTVDKSRWQQGNVFSCSVMHEALHNHYTQKSLSLSPGGGGSYRYRQGG |
| | | GS |
| 99 | IgG1 hinge CH2- | DKTHTCPPCPAPELLGGPSVFLFPPKPKDTLMISRTPEVTCVVVDVSHEDPEVKFNWYVD |
| | CH3 (407V, | GVEVHNAKTKPREEQYNSTYRVVSVLTVLHQDWLNGKEYKCKVSNKALPAPIEKTISKA |
| | 366S, 368A, | KGQPREPQVCTLPPSRDELTKNQVSLSCAVKGFYPSDIAVEWESNGQPENNYKTTPPVLD |
| | 349C) + Q-tag (C | SDGSFFLVSKLTVDKSRWQQGNVFSCSVMHEALHNHYTQKSLSLSPGGGGSYRYRQGGG |
| | term) | S |
| 100 | IgG1 hinge CH2- | GLNDIFEAQKIEWHEGGDKTHTCPPCPAPELLGGPSVFLFPPKPKDTLMISRTPEVTCVVV |
| | CH3 (366W, | DVSHEDPEVKFNWYVDGVEVHNAKTKPREEQYNSTYRVVSVLTVLHQDWLNGKEYKC |
| | 354C) + Avi-tag | KVSNKALPAPIEKTISKAKGQPREPQVYTLPPCRDELTKNQVSLWCLVKGFYPSDIAVEW |
| | (N term) | ESNGQPENNYKTTPPVLDSDGSFFLYSKLTVDKSRWQQGNVFSCSVMHEALHNHYTQKS |
| | | LSLSPGK |
| 101 | IgG1 hinge CH2- | GLNDIFEAQKIEWHEGGDKTHTCPPCPAPELLGGPSVFLFPPKPKDTLMISRTPEVTCVVV |
| | CH3 (407V, | DVSHEDPEVKFNWYVDGVEVHNAKTKPREEQYNSTYRVVSVLTVLHQDWLNGKEYKC |
| | 366S, 368A, | KVSNKALPAPIEKTISKAKGQPREPQVCTLPPSRDELTKNQVSLSCAVKGFYPSDIAVEWE |
| | 349C) + Avi-tag | SNGQPENNYKTTPPVLDSDGSFFLVSKLTVDKSRWQQGNVFSCSVMHEALHNHYTQKSL |
| | (N term) | SLSPGK |
| 102 | IgG1 hinge CH2- | DKTHTCPPCPAPELLGGPSVFLFPPKPKDTLMISRTPEVTCVVVDVSHEDPEVKFNWYVD |
| | CH3 (366W, | GVEVHNAKTKPREEQYNSTYRVVSVLTVLHQDWLNGKEYKCKVSNKALPAPIEKTISKA |
| | 354C) + Avi-tag | KGQPREPQVYTLPPCRDELTKNQVSLWCLVKGFYPSDIAVEWESNGQPENNYKTTPPVL |
| | (C term) | DSDGSFFLYSKLTVDKSRWQQGNVFSCSVMHEALHNHYTQKSLSLSPGKGGGLNDIFEA |
| | | QKIEWHE |
| 103 | IgG1 hinge CH2- | DKTHTCPPCPAPELLGGPSVFLFPPKPKDTLMISRTPEVTCVVVDVSHEDPEVKFNWYVD |
| | CH3 (407V, | GVEVHNAKTKPREEQYNSTYRVVSVLTVLHQDWLNGKEYKCKVSNKALPAPIEKTISKA |
| | 366S, 368A, | KGQPREPQVCTLPPSRDELTKNQVSLSCAVKGFYPSDIAVEWESNGQPENNYKTTPPVLD |
| | 349C) + Avi-tag | SDGSFFLVSKLTVDKSRWQQGNVFSCSVMHEALHNHYTQKSLSLSPGKGGGLNDIFEAQ |
| | (C term) | KIEWHE |

### DESCRIPTION OF THE FIGURES

FIG. 1: Schematic illustrating the payload-attaching chain exchange concept. A set of one-5 armed antibodies is exemplarily shown. Different binders have varying sequences in the Fab region. On the upper right (circled) a single conjugation reaction with an Fc-only-dimer is depicted. The conjugated donor molecule is then applied to the exchange reaction with each of the different binders (acceptors). After the chain exchange the mixture is applied to an affinity C-tag column that binds all dummy dimers, aggregates or non-reacted educt molecules containing the C-tag. The product, defined labelled antibodies, is present in the flowthrough at high product purity.

FIGS. 2A to 2D: Schematics, graphs, image and bar chart relating to chain exchange-mediated attachment of dyes from NHS-lysine conjugated donor modules. (FIG. 2A) Two reactant molecules are mixed in equimolar concentrations in PBS under mild reducing conditions (20-fold molar access TCEP). The mixture is kept at 37° C. for 3 hours while shaking. The reaction is then loaded onto a C-tag affinity chromatography and the flow through is collected. (FIG. 2B) The labelled product has a purity of ≥98%, the capillary electrophoresis (CE-SDS) reveals defined bands of expected sizes under both (non-) reducing conditions. HC=heavy chain, LC=light chain, Ab=antibody. (FIG. 2C) Exemplarily two different formats were used, a Fab-Fc molecule containing CH1 and Ckappa domains, and a format with scFv as targeting entity (VH+VL only). (FIG. 2D) Two binders (C6.5, F5) in different formats (Fab-Fc and scFv-Fc) and a positive control 4D5-8 (Trastuzumab-derived, in Fab-Fc format) were processed. All molecules carry the same DAR without significant differences in labelling. As expected, the payload-Fc input molecule has more than twice as much payload attached.

FIGS. 3A and 3B: Schematic, graphs, and bar chart relating to internalisation capabilities of different Her2 binders. Robust Chain exchange-mediated attachment of dyes from NHS-lysine conjugated donor modules. (FIG. 3A) To validate the concept five different clones/formats were labelled with a pH-dependent dye (pHab). The products were applied to Her2-positive cell lines. Following internalisation the dye accumulates and gains activity in the acidic environment of the endosomes. Thereby antibody-mediated internalisation can be measured. (FIG. 3B) All binders internalise as reflected by an increased PE-signal compared to the isotype control. Absolute internalisation (intracellular amount of dye) and relative internalisation (ratio to the binding signal) is shown.

FIG. 4: Schematic and image relating to chain exchange-mediated attachment of horseradish peroxidase. Horseradish peroxidase (HRP) was attached to the EGFR-binding antibody C225. The western blot indicates the suitability of this binder to detect denatured (SDS-PAGE) EGFR, and the activity of the attached enzyme.

FIGS. 5A and 5B: Schematics, image and bar chart relating to transfer of payloads conjugated to donor Fcs in a site-specific manner. Robust Chain exchange-mediated attachment of dyes from NHS-lysine conjugated donor modules. (FIG. 5A) Biotin was conjugated to the Fc-precursor molecule via site-directed AviTag biotinylation. The functionality of the biotinylated antibody obtained after chain exchange was demonstrated by its application in immunoprecipitation of EGFR from whole A431 cell lysate (magnetic streptavidin beads). (FIG. SB) Ruthenium was conjugated to an Fc-precursor molecule carrying a C-terminal K-tag. Q-tag carrying Ruthenium was attached to the K-tag of the donor Fc by transglutaminase. The functionality of the Ruthenium-conjugated antibody obtained after chain exchange was demonstrated by its application in an electrochemiluminescence assay (Elecsys/Cobas).

FIG. 6: Schematic and graphs relating to antibody-payload fusion proteins generated by chain exchange-mediated attachment. The EGFR-binding acceptor molecule based on the C225 (cetuximab) sequence was subjected to chain exchange with a donor chain containing a GFP fusion protein at the C-terminus. FACS analyses demonstrate specific binding to EGFR expressing A431 cells, competition experiments with other published antibodies (PIX, P2X and mab806) reveal EGFR domain III as target of C225 which agrees with published data (Li et al. 2005).

### EXAMPLES

### Example 1: Methods

### 1.1 Expression and Purification of Precursor Molecules

Expression plasmids encoded CMV-promoter transcribed recombinant antibody derivatives that become secreted into cell culture supernatants as previously described (Dengl et al.). The transient HEK-293-Expi system (Thermo Fisher) was used according to the manufacturer's instructions. Cultures were maintained for 6 days at 37° C. in humidified 8% CO2 atmosphere. All proteins described herein were secreted into the culture supernatant (IgG VH secretion leader peptides). Supernatants were separated from cellular content by centrifugation (3,500 g, 45 min) and filtration (0.22 μm). Antibody derivatives were then enriched using a ProtA (HiTrap™ Protein A HP, GE, Cat. No. 28989336, Boston, MA, USA) affinity chromatography and size exclusion chromatography (HiLoad® 26/600 Superdex® 200, GE, Cat. No. 28989336, Boston, MA, USA),

The polypeptides employed in the experimental examples of the present disclosure are shown in SEQ ID NOs: 72 to 103. The following polypeptides were employed:

Acceptor precursor molecules:

TABLE 1
Fab-Fc specifically binding to Her2 (4D5)

| | | | heavy | heavy |
|---|---|---|---|---|
| | dummy c | light | chain | chain |
| | tag with | chain | (hole) | (knob) |
| Trastuzumab[Her2_4D5- | knob | SEQ ID | SEQ ID | SEQ ID |
| 8]-hole (n) + knob- | | NO: 72 | NO: 73 | NO: 74 |
| dummy_C-Tag (C354S - | | | | |
| K370E) | | | | |
| Trastuzumab[Her2_4D5- | hole | SEQ ID | SEQ ID | SEQ ID |
| 8]-knob (n) + hole- | | NO: 72 | NO: 76 | NO: 75 |
| dummy_C-Tag (C349Y - | | | | |
| E357K) | | | | |

TABLE 2
Fab-Fc and scFv-Fc specifically binding to Her2 (F5)

| | | | heavy | heavy |
|---|---|---|---|---|
| | dummy c | light | chain | chain |
| | tag with | chain | (hole) | (knob) |
| Her2_F5-hole (n) + | knob | SEQ ID | SEQ ID | SEQ ID |
| knob-dummy_C-Tag | | NO: 77 | NO: 78 | NO: 74 |
| (C354S - K370E) | | | | |
| | | scFv | | heavy chain |
| | | (hole) | | (knob) |
| Her2_F5-hole (scFv) + | knob | SEQ ID | | SEQ ID |
| knob-dummy_C- | | NO: 79 | | NO: 74 |
| Tag (C354S - K370E) | | | | |

TABLE 3
Fab-Fc and scFv specifically binding to Her2 (C6.5)

| | | | heavy | heavy |
|---|---|---|---|---|
| | dummy c | light | chain | chain |
| | tag with | chain | (hole) | (knob) |
| Her2_C6.5-hole (n) + | knob | SEQ ID | SEQ ID | SEQ ID |
| knob-dummy_C-Tag | | NO: 80 | NO: 81 | NO: 74 |
| (C354S - K370E) | | | | |
| | | scFv | | heavy chain |
| | | (hole) | | (knob) |
| Her2_C6.5-hole | knob | SEQ ID | | SEQ ID |
| (scFv) + knob- | | NO: 82 | | NO: 74 |
| dummy_C-Tag | | | | |
| (C354S - K370E) | | | | |

TABLE 4
Fab-Fc and scFv specifically binding to Her1 (C225)

| | | | heavy | heavy |
|---|---|---|---|---|
| | dummy c | light | chain | chain |
| | tag with | chain | (hole) | (knob) |
| Cetuximab[Her1_C225]- | knob | SEQ ID | SEQ ID | SEQ ID |
| hole (n) + knob- | | NO: 83 | NO: 84 | NO: 74 |
| dummy_C-Tag (C354S - | | | | |
| K370E) | | | | |
| Cetuximab[Her1_C225]- | hole | SEQ ID | SEQ ID | SEQ ID |
| knob (n) + hole- | | NO: 83 | NO: 75 | NO: 85 |
| dummy_C-Tag (C349Y - | | | | |
| E357K) | | | | |

Donor precursor molecules:

TABLE 5
dummy-Fc with C-tag

| | hole | knob | dummy c |
|---|---|---|---|
| | polypeptide | polypeptide | tag with |
| hole-dummy_C-Tag | SEQ ID | SEQ ID | hole |
| (C349Y - E357K)/hinge- | NO: 75 | NO: 86 | |
| Fc-knob | | | |
| knob-dummy_C-Tag | SEQ ID | SEQ ID | knob |
| (C354S - K370E)/hinge- | NO: 87 | NO: 74 | |
| Fc-hole | | | |

TABLE 6
dummy-Fc with LALAPG mutations and C-tag

| | hole | knob | dummy c |
|---|---|---|---|
| | polypeptide | polypeptide | tag with |
| hole-dummy_C-Tag | SEQ ID | SEQ ID | hole |
| (C349Y - E357K)/hinge- | NO: 88 | NO: 89 | |
| Fc-knob (LALAPG) | | | |
| knob-dummy_C-Tag | SEQ ID | SEQ ID | knob |
| (C354S - K370E)/hinge- | NO: 91 | NO: 90 | |
| Fc-hole (LALAPG) | | | |

TABLE 7
dummy-Fc with C-tag labelled with EGFP

| | hole | knob | dummy c |
|---|---|---|---|
| | polypeptide | polypeptide | tag with |
| hole-dummy_C-Tag | SEQ ID | SEQ ID | hole |
| (C349Y - E357K)/hinge- | NO: 75 | NO: 92 | |
| Fc-knob_mEGFP(A206K) | | | |
| knob-dummy_C-Tag | SEQ ID | SEQ ID | knob |
| (C354S - K370E)/hinge- | NO: 74 | NO: 93 | |
| Fc-hole_mEGFP(A206K) | | | |

TABLE 8
dummy-Fc with C-tag labelled with Q-tag

| | hole | knob | dummy c |
|---|---|---|---|
| | polypeptide | polypeptide | tag with |
| hole-dummy_C-Tag | SEQ ID | SEQ ID | hole |
| (C349Y - E357K)/hinge- | NO: 75 | NO: 94 | |
| Fc-knob_Q-Tag (297) | | | |
| knob-dummy_C-Tag | SEQ ID | SEQ ID | knob |
| (C354S - K370E)/hinge- | NO: 95 | NO: 74 | |
| Fc-hole_Q-Tag (297) | | | |

TABLE 9
dummy-Fc with LALAPG mutations and C-tag labelled with Q-tag

| | hole | knob | dummy c |
|---|---|---|---|
| | polypeptide | polypeptide | tag with |
| hole-dummy_C-Tag | SEQ ID | SEQ ID | hole |
| (C349Y - E357K)/hinge- | NO: 88 | NO: 96 | |
| Fc-knob_Q-Tag (297) | | | |
| [LALAPG] | | | |
| knob-dummy_C-Tag | SEQ ID | SEQ ID | knob |
| (C354S - K370E)/hinge- | NO: 97 | NO: 90 | |
| Fc-hole_Q-Tag (297) | | | |
| [LALAPG] | | | |

TABLE 10
dummy-Fc with C-tag labelled with C-terminal Q-tag

| | hole | knob | dummy c |
|---|---|---|---|
| | polypeptide | polypeptide | tag with |
| hole-dummy_C-Tag | SEQ ID | SEQ ID | hole |
| (C349Y - E357K)/hinge- | NO: 75 | NO: 98 | |
| Fc-knob_Q-Tag (C-term.) | | | |
| knob-dummy_C-Tag | SEQ ID | SEQ ID | knob |
| (C354S - K370E)/hinge- | NO: 99 | NO: 74 | |
| Fc-hole_Q-Tag (C-term.) | | | |

TABLE 11
dummy-Fc with C-tag labelled with N-terminal Avi-tag

| | hole | knob | dummy c |
|---|---|---|---|
| | polypeptide | polypeptide | tag with |
| hole-dummy_C-Tag | SEQ ID | SEQ ID | hole |
| (C349Y - E357K)/hinge- | NO: 75 | NO: 100 | |
| Fc-knob_Avi-Tag | | | |
| (N-term.) | | | |
| knob-dummy_C-Tag | SEQ ID | SEQ ID | knob |
| (C354S - K370E)/hinge- | NO: 101 | NO: 74 | |
| Fc-hole_Avi-Tag | | | |
| (N-term.) | | | |

TABLE 12
dummy-Fc with C-tag labelled with C-terminal Avi-tag

| | hole | knob | dummy c |
|---|---|---|---|
| | polypeptide | polypeptide | tag with |
| hole-dummy_C-Tag | SEQ ID | SEQ ID | hole |
| (C349Y - E357K)/hinge- | NO: 75 | NO: 102 | |
| Fc-knob_Avi-Tag | | | |
| (C-term.) | | | |
| knob-dummy_C-Tag | SEQ ID | SEQ ID | knob |
| (C354S - K370E)/hinge- | NO: 103 | NO: 74 | |
| Fc-hole_Avi-Tag | | | |
| (C-term.) | | | |

### 1.2 Payload Attachment to Exchange-Enabled Precursor Molecules

Fc-only precursor molecules were coupled to payloads to serve as payload donor entities. This was achieved by (i) site-directed fusion of enhanced GFP (UniProt) to the C-terminal end including a G4S-Linker, (ii) site-directed conjugation of a biotin via the AviTag technology (BirA Bulk Kit, Avidity, manufacturer's instructions), (iii) random amine-reactive labelling with a pH sensitive dye (Promega, Cat. No. G9845, manufacturer's instructions including the DAR calculation), (iv) random amine reactive labelling with horseradish peroxidase (EZ-Link® Activated Peroxidase Antibody Labeling Kit, Thermo Scientific, Cat. No. 31497, manufacturer's instructions). Chemical or enzymatic coupling reactions were according to the manufacturer's protocols.

### 1.3 Donor-to-Acceptor Transfer of the Payload by Chain Exchange

In order to facilitate polypeptide chain exchange between a donor precursor molecule and an acceptor precursor molecule, donor prescursors comprising a knob-dummy polypeptide are mixed with acceptor precursors comprising the corresponding hole-dummy polypeptide, while prescursors comprising a hole-dummy polypeptide are mixed with acceptor precursors comprising the corresponding knob-dummy polypeptide.

Donor and acceptor precursor complexes were mixed in equimolar amounts at 1 mg/ml concentration in PBS, pH 7.4. A molar excess of 20-fold TCEP was added to reduce the disulfide bridge of the hinge regions to initiate the exchange reaction. The mix was kept for 3 hours at 37° C. and 300 rpm. Resulting exchange products were purified by absorbing aggregates, unreacted precursors and dummy dimers via their C-tag on an affinity column (CaptureSelect™ C-tagXL Pre-packed Column/Thermo Scientific, Cat. No. 494307205). Analytical SEC (Superdex 200 analytical size-exclusion column GE Healthcare, Sweden) in 200 mM KH₂PO₄, 250 mM KCl, pH 7.0 running buffer at 25° C., and capillary electrophoresis (CE-SDS, Caliper Life Sciences) was applied to assess composition and quality of the products.

### 1.4 Binding and Internalisation Assays

Binding to target cells was assessed flow cytometry. A total of 3×10⁵ cells were incubated with 200 nM of antibody derivatives in FACS buffer (PBS with 2% FCS) for 1 h at 37° C. Then, cells were washed twice with PBS and analysed for phycoerythrin intensity using a FACS CantoII instrument (BD biosciences). FloJo (BD) software was used for data analysis and visualisation. For epitope binning assays, FACS analyses were performed as above with addition of 200 nM competing IgG (P1X, P2X and mab806) that bind known epitopes on EGFR domains I, II and III were added in separate wells at. FITC intensity was measured using the FACS Canto II instrument. To study internalisation, 1×10⁵ SK-BR-3 cells (ATCC) were seeded out in flat-bottom 96 well plates and treated with 500 nM of the respective Her-2-binding clone with pHAb label in a final volume of 200 μl. Mixture was kept for 24 hours at 37° C., humidified 8% CO2 atmosphere. Cells were then detached with 100 uL Accutase (Pan Biotech, Cat. No. P10-21100), washed twice with PBS and analysed via the cytometer as mentioned above. For visualisation of bar graphs GraphPad Prism 7 software (GraphPad Software, San Diego, CA, USA) was used.

### 1.5 Immunoprecipitation and Western Blot

3×10⁶ A431 cells were resuspended on ice in 1 ml RIPA Lysis and Extraction Buffer (Thermo Scientific, Cat. No. 89900). Cell debris was removed by centrifugation at 15000 g for 15 min and supernatant collected. Pierce™ Streptavidin Magnetic Beads (Thermo Scientific, Cat. No. 88816, applied according to the manual) was used to pulldown EGFR molecules with the biotinylated C225 antibody. The elution from the beads was done with 50 μl NuPAGE™ LDS Sample Buffer (Thermo-Fisher, Cat. No. NP0007), 5 min at 95° C. After removal of magnetic beads, 20 μl were loaded onto a gel 4 to 12% Bis-Tris Gel (Invitrogen, Cat. No. NP0322BOX), run for 60 minutes at 180 V. Proteins were blotted using the Trans-Blot Turbo Mini 0.2 μm PVDF Transfer Packs (Bio-Rad, Cat. No. 1704156) and the Trans Blot Turbo Transfer System (Bio-Rad) according to the manufacturer's instructions for high-molecular weight species. The membrane was then blocked for 30 minutes in blocking buffer (1×TBS, 0.05% Tween20, 5% skim milk). The membrane was then incubated overnight in blocking buffer containing anti-EGFR primary antibody (Abcam, ab264540, 1:1000). Next day, the membrane was washed 3×5 min with TBS-T and incubated with secondary antibody (Polyclonal Goat anti-Mouse Immunoglobulins/HRP; Agilent Dako, Cat. No. P044701-2, 1:1500) in blocking buffer for 1 hour at RT. After three rounds of TBS-T wash, chemiluminescence was detected using SuperSignal™ West Pico PLUS Chemiluminescent Substrate (Thermo Scientific, Cat. No. 34579) and the Gel Doc XR+Gel Documentation System. For testing the functionality of C225-HRP, the Western Blot procedure was done similarly. The A431 lysate was loaded onto the gel, blotting was done accordingly, C225-HRP was then applied at a concentration of c=0,24 μg/mL in blocking buffer and the above mentioned readout was used.

### Example 2: Results and Discussion

### 2.1 Payload-Redirecting Chain Exchange

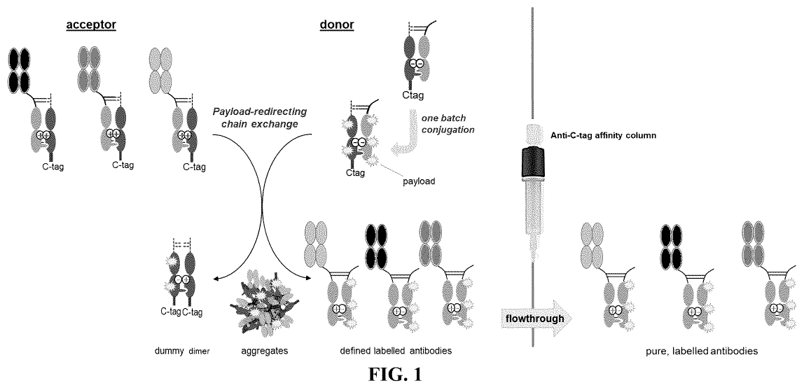

The principle of the payload-redirecting chain exchange concept is depicted in FIG. 1. Different binder modules (black, dark grey, light grey) are exchange-enabled by carrying partially destabilised knob-into-hole CH3 interfaces with repulsive charges as previously described by Dengl et al. 2020. These derivatives with different binding (Fab/Fv) regions represent acceptor entities for payloads to be attached. The payload donor entities are complementary (also partially destabilised) knob-into-hole Fc-only-precursors, which are conjugated to one or more payloads, either in a site-directed or random manner.

Mixing the acceptor and donor entities under slightly reducing conditions for limited reduction of hinge disulfides (see Materials & Methods section of Dengl et al. 2020) triggers an exchange reaction driven by the repulsive charges in the interface of the precursors. That reaction attaches the payload to the binder, with dummy-dimers as by-products. Dummy-dimers, as well as donor and acceptor precursors (but not the ADC products) harbour C-terminal C-tags and can be removed from the reaction mixture by adsorption on a C-Tag affinity column. The flowthrough of those columns contains the payload-coupled binders. Due to the fact that the exchange reaction transfers one defined conjugate as donor to sets of many different acceptor binders, conjugation-mediated variabilities between the resulting ADCs are excluded. All products carry different binders, but with identical payloads and drug-to-antibody ratio (DAR), attached to the same positions of the ADCs. Exchange-mediated defined payload attachment can be performed with various payloads attached to donor modules by different means. This includes ‘random’ attachment to donors (thereafter converted to defined ADC products), and attachment by site-directed payload conjugation or fusion to donor entities as described hereafter.

2.2 Defined ADC Sets Generated from Random NHS-Conjugated Payload Donor Modules

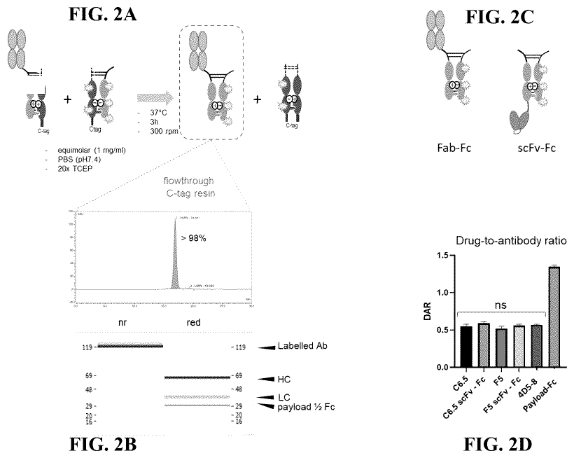

Conjugation of NHS-modified payloads to lysine residues exposed on protein surfaces is a robust 1^{st} generation workhorse technology for ADC generation. That type of conjugation can be considered ‘random’ because multiple lysines are scattered on antibody surfaces, and in some cases right at or in proximity to antigen binding sites. It is therefore challenging to generate sets of antibodies with different sequences of which all molecules retain full binding functionalities and are conjugated to NHS-payloads in a comparable manner (DAR & positions). The chain exchange technology overcomes the common obstacles of heterogeneous conjugation patterns and potentially compromised binding regions. FIGS. 2A-2D show how NHS-conjugation (with payloads randomly assigned to donor-Fc), is converted by exchange-mediated attachment to sets of different antibodies with identical payloads (DAR) and unmodified binding regions. In this two-step method, the payload is first conjugated to the exchange donor rather than directly fused to the antibody of interest. The transfer of the exchange module in the second step is then constant among all antibodies (acceptors) of interest to which payloads shall be attached.

As an example for that approach, different Her2-targeting acceptor molecules were mixed at equimolar concentrations with an Fc-only donor molecule that was previously labelled with a pH sensitive dye by random conjugation to reactive amine groups. The reaction buffer was standard PBS with a 20-fold molar access of TCEP (reducing agent) to break up hinge-disulfides. After 3 hours at 37° C. and orbital shaking at 300 rpm, the mix was applied to a C-tag resin. The flowthrough samples contained the labelled product with high purity (≥95%) and with the expected composition (FIG. 2B). In order to show, that the reaction is neither affected by the binder sequence, nor by the format (FIG. 2C) of the target-binding acceptor, three different Her2-binders and two different formats of the Her2 binders were compared. FIG. 2D shows that all binders, in all formats (Fab-Fc containing CH1 and Ck; scFv-Fc) become labelled to the same degree. All acceptor molecules carry the same exchange interface, in consequence the DAR was the same in all five examples. FIG. 2D also reveals that the initial donor payload-Fc precursor carried approximately twice as many fluorophores than found in the products. This confirms the mode of the exchange reaction by which half of the donor molecule is transferred to the acceptor, while the other half ends up in the dummy-dimer by-product.

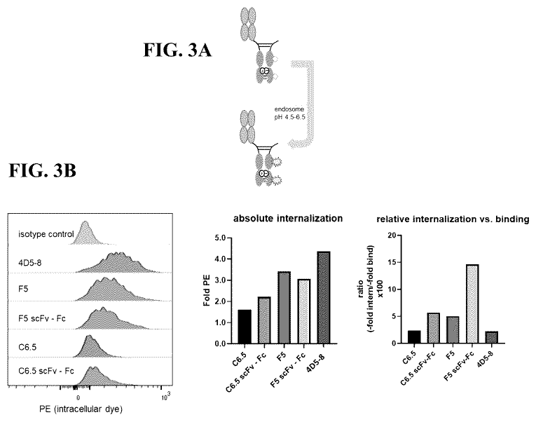

The generation of sets of ADC with identical labels and DAR, while assuring uncompromised binding regions, is important if one wants to compare the functionality and/or applicability of different antibodies. One parameter known to modulate the efficacy of ADCs with cytotoxic payloads is the capability to internalise and thereby deliver payloads into target cells (Maass et al. 2016; Tang et al. 2019). The pH-sensitive dye attached to the different Her2-binders (FIG. 2A-2D) lights up upon internalisation into cells and trafficking into endo-/lysosomal pathway where it encounters an acidic environment (FIG. 3A). The prerequisites for applying ADCs with this dye to compare and quantify internalisation is that different binders harbour the dye in a comparable manner and without affecting their binding regions. This is difficult to achieve by individual NHS-conjugations, but is guaranteed by our simple and robust chain-exchange procedure. The applicability of the thereby produced ADCs for a comparison of the internalisation of different Her2 binders and formats is shown in FIGS. 3A-3B.

2.3 Defined Antibody-Enzyme Conjugates Generated from Random Conjugated Donor Modules

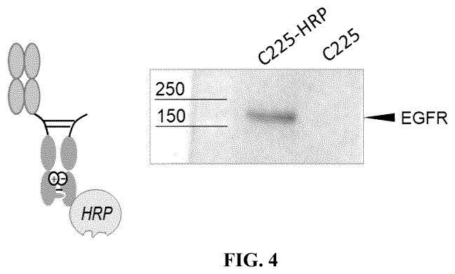

Conversion of random conjugates to rather defined molecules with free binding regions can be achieved not only for small molecular weight payloads such as dyes, but also for attaching large molecules, i.e. proteins. FIG. 4 shows an NHS-lysine conjugate of the enzyme horseradish peroxidase (HRP) to the donor precursor. The subsequent exchange reaction attaches enzyme-carrying donor halves to the acceptor molecule, in this case an EGFR-binding antibody (clone C225). The combined functionality of both enzyme and the binding region was demonstrated by applying the conjugate as Western Blot detection reagent. FIG. 4 shows that the resulting anti-EGFR-HRP conjugate detects EGFR in total blotted cell lysates of A431 cells with high specificity and low background. Thus, chain exchange mediated ADC generation is not limited to attachment of small compounds but is also applicable to large molecule payloads.

2.4 ADC Generation by Payload Transfer from Site-Specific Conjugated Donor Modules

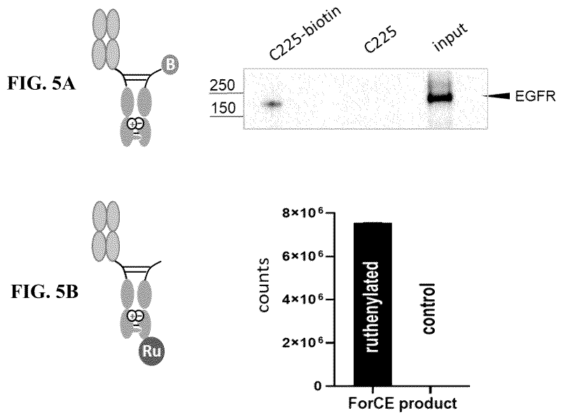

The chain exchange technology is not limited to conversion of random NHS conjugates to defined ADCs with uncompromised binding regions. It also enables the effective transfer of payloads that are attached to donors in a site-directed manner to sets of different binder acceptors. Examples for such applications with donors that carry site-specific modifications are biotinylation of antibody derivatives, and transglutaminase-mediated attachment of labels. FIGS. 5A-5B shows donor Fc parts that carry N- or C-terminally fused AviTags for site specific (via birA, see M&M) biotinylation. These can be transferred to accepting binders resulting in defined mono-(or bi-) biotinylated antibodies with intact antigen binding regions.

Those biotinylated antibody derivatives are defined reagents that can be used for various assays such as binder immobilisation (e.g. on SPR chips), antigen capture, and serve as versatile assay reagents. One application example (antigen pulldown) is shown in FIG. 5A.

Transglutaminase-mediated attachment is another method for enzyme-mediated site specific conjugation compatible with our exchange technology. FIG. 5B shows an example in which Ruthenium was attached to a C-terminal positioned Transglutaminase site (K-tag on Ruthenium to Q-tag on Fc, see M&M) on donor Fc's. Chain exchange is then a simple and robust means to generate from one ‘stock reagent’ many Ruthenium-labelled antibody derivatives that serve as sensitive detection reagents in electrochemiluminescence (ECL)-based assays (FIG. 5B).

### 2.5 Antibody-Payload Fusion Proteins Generated by Chain Exchange Mediated Attachment

The chain exchange technology is not restricted to attachment of chemically or enzyme-linked payloads. Payloads can also be placed in a site-specific manner to donor Fcs as fusions. The fused Fc is then transferred to the binding acceptor, resulting in ‘ADC-like’ antibody-payload combinations. This application is particularly suited to attach defined fusion partners in a high throughput manner to many different antibodies. To highlight that versatility of the concept we produced the payload donor in form of a C-terminal fusion with enhanced GFP (˜30 kDa). This donor molecule was well behaved (soluble, low aggregation) and could be expressed with good yields similar to regular antibodies. This protein can hence be produced as a universal ‘stock reagent’ for exchange-mediated attachment to any exchange-enabled antibody derivative. The GFP fusion served as payload transfer donor in the same manner as random- or site-specific donor-payload conjugates. Thus, the fusion-protein format did not interfere with the exchange and resulted in effective transfer of the fluorescent protein to the acceptor molecule.

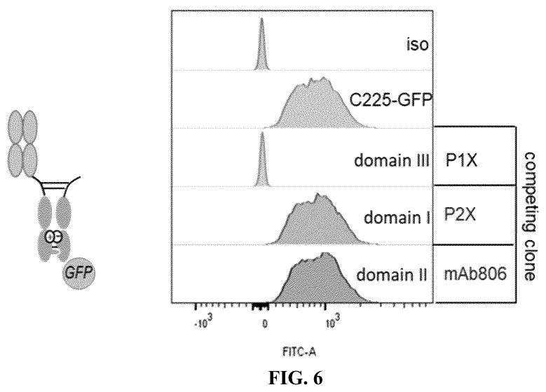

An example for payload transfer in form of fusion proteins is shown in FIG. 6. An EGFR-binding acceptor module (C225/Cetuximab derived sequence) was subjected to exchange for generation of GFP-fused derivatives, which were then used in FACS experiments.

The results of these analyses demonstrate that such molecules are applicable as specific detection reagents in assays with fluorescence readout, incl. in FACS analyses (FIG. 6). The exchange reaction can generate fluorescent fusion protein derivatives of different antibodies in a simple and robust manner. A common application that requires sets of comparably labelled different antibodies directed at the same antigen is ‘epitope binning’ (Abdiche et al. 2012). FIG. 6 shows that exchange-generated antibody-GFP fusions can be applied for such tasks, demonstrating the suitability of the concept for epitope binning applications.

### REFERENCES

Abdiche, Y. N., K. C. Lindquist, D. M. Stone, A. Rajpal, and J. Pons. 2012. ‘Label-free epitope binning assays of monoclonal antibodies enable the identification of antigen heterogeneity’, J Immunol Methods, 382:101-16.
Akkapeddi, Padma, Saara-Anne Azizi, Allyson M. Freedy, Pedro M. S. D. Cal, Pedro M. P. Gois, and Gonçalo J. L. Bernardes. 2016. ‘Construction of homogeneous antibody-drug conjugates using site-selective protein chemistry’, Chemical science, 7:2954-63.
Beck, Alain, Liliane Goetsch, Charles Dumontet, and Nathalie Corvaïa. 2017. ‘Strategies and challenges for the next generation of antibody-drug conjugates’, Nature Reviews Drug Discovery, 16:315-37.
Dengl, S., K. Mayer, F. Bormann, H. Duerr, E. Hoffmann, B. Nussbaum, M. Tischler, M. Wagner, A. Kuglstatter, L. Leibrock, C. Buldun, G. Georges, and U. Brinkmann. 2020. ‘Format chain exchange (FORCE) for high-throughput generation of bispecific antibodies in combinatorial binder-format matrices’, Nat Commun, 11:4974.
Hussain, Ahmad Fawzi, Armin Grimm, Wenjie Sheng, Chaoyu Zhang, Marwah Al-Rawe, Karen Bräutigam, Mobarak Abu Mraheil, Felix Zeppernick, and Ivo Meinhold-Heerlein. 2021. ‘Toward Homogenous Antibody Drug Conjugates Using Enzyme-Based Conjugation Approaches’, Pharmaceuticals, 14:343.
Li, Shiqing, Karl R. Schmitz, Philip D. Jeffrey, Jed J. W. Wiltzius, Paul Kussie, and Kathryn M. Ferguson. 2005. ‘Structural basis for inhibition of the epidermal growth factor receptor by cetuximab’, Cancer Cell, 7:301-11.
Maass, Katie F., Chethana Kulkarni, Alison M. Betts, and K. Dane Wittrup. 2016. ‘Determination of Cellular Processing Rates for a Trastuzumab-Maytansinoid Antibody-Drug Conjugate (ADC) Highlights Key Parameters for ADC Design’, The AAPS journal, 18:635-46.
Möhlmann, S., P. Bringmann, S. Greven, and A. Harrenga. 2011. ‘Site-specific modification of ED-B-targeting antibody using intein-fusion technology’, BMC Biotechnol, 11:76.
Nath, N., B. Godat, C. Zimprich, S. J. Dwight, C. Corona, M. McDougall, and M. Urh. 2016. ‘Homogeneous plate based antibody internalisation assay using pH sensor fluorescent dye’, J Immunol Methods, 431:11-21.
Patterson, James T., Shigehiro Asano, Xiuling Li, Christoph Rader, and Carlos F. Barbas, 3rd. 2014. ‘Improving the serum stability of site-specific antibody conjugates with sulfone linkers’, Bioconjugate chemistry, 25:1402-07.
Sadiki, Amissi, Shefali R Vaidya, Mina Abdollahi, Gunjan Bhardwaj, Michael E Dolan, Harpreet Turna, Varnika Arora, Athul Sanjeev, Timothy D Robinson, Andrea Koid, Aashka Amin, and Zhaohui Sunny Zhou. 2020. ‘Site-specific conjugation of native antibody’, Antibody Therapeutics, 3:271-84.
Steffen, W., F. C. Ko, J. Patel, V. Lyamichev, T. J. Albert, J. Benz, M. G. Rudolph, F. Bergmann, T. Streidl, P. Kratzsch, M. Boenitz-Dulat, T. Oelschlaegel, and M. Schraeml. 2017. ‘Discovery of a microbial transglutaminase enabling highly site-specific labeling of proteins’, J Biol Chem, 292:15622-35.
Strop, Pavel, Shu-Hui Liu, Magdalena Dorywalska, Kathy Delaria, Russell G Dushin, Thomas-Toan Tran, Wei-Hsien Ho, Santiago Farias, Meritxell Galindo Casas, Yasmina Abdiche, Dahui Zhou, Ramalakshmi Chandrasekaran, Caroline Samain, Carole Loo, Andrea Rossi, Mathias Rickert, Stellanie Krimm, Teresa Wong, Sherman Michael Chin, Jessica Yu, Jeanette Dilley, Javier Chaparro-Riggers, Gary F Filzen, Christopher J O'Donnell, Fang
Wang, Jeremy S Myers, Jaume Pons, David L Shelton, and Arvind Rajpal. 2013. ‘Location Matters: Site of Conjugation Modulates Stability and Pharmacokinetics of Antibody Drug Conjugates’, Chemistry & Biology, 20:161-67.
Su, Dian, and Donglu Zhang. 2021. ‘Linker Design Impacts Antibody-Drug Conjugate Pharmacokinetics and Efficacy via Modulating the Stability and Payload Release Efficiency’, Frontiers in Pharmacology, 12.
Sun, X., J. F. Ponte, N. C. Yoder, R. Laleau, J. Coccia, L. Lanieri, Q. Qiu, R. Wu, E. Hong, M. Bogalhas, L. Wang, L. Dong, Y. Setiady, E. K. Maloney, O. Ab, X. Zhang, J. Pinkas, T. A. Keating, R. Chari, H. K. Erickson, and J. M. Lambert. 2017. ‘Effects of Drug-Antibody Ratio on Pharmacokinetics, Biodistribution, Efficacy, and Tolerability of Antibody-Maytansinoid Conjugates’, Bioconjugate chemistry, 28:1371-81.
Tang, Haichao, Yan Liu, Zhaojin Yu, Mingli Sun, Lu Lin, Wensi Liu, Qiang Han, Minjie Wei, and Ying Jin. 2019. ‘The Analysis of Key Factors Related to ADCs Structural Design’, Frontiers in Pharmacology, 10.
Tsuchikama, Kyoji, and Zhiqiang An. 2018. ‘Antibody-drug conjugates: recent advances in conjugation and linker chemistries’, Protein & Cell, 9:33-46.

1. A method for producing a polypeptide complex, comprising:
incubating:
(1) a first polypeptide complex, comprising a first polypeptide comprising a CH3 domain and a second polypeptide comprising a CH3 domain;
wherein the first polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide comprises a knob modification, and the CH3 domain of the second polypeptide comprises a hole modification;
wherein the CH3 domain of the first polypeptide or the CH3 domain of the second polypeptide comprises a destabilising modification for destabilising association between the CH3 domain of the first polypeptide and the CH3 domain of the second polypeptide;
and wherein the first polypeptide and/or the second polypeptide further comprise a payload moiety; and
(2) a second polypeptide complex, comprising a third polypeptide comprising a CH3 domain and a fourth polypeptide comprising a CH3 domain;
wherein the second polypeptide complex is formed by interaction between the third polypeptide and the fourth polypeptide, the interaction comprising association between the CH3 domain of the third polypeptide and the CH3 domain of the fourth polypeptide;
wherein the CH3 domain of the third polypeptide comprises a knob modification, and the CH3 domain of the fourth polypeptide comprises a hole modification;
wherein the CH3 domain of the third polypeptide or the CH3 domain of the fourth polypeptide comprises a destabilising modification for destabilising association between the CH3 domain of the third polypeptide and the CH3 domain of the fourth polypeptide;
to form a third polypeptide complex comprising the first polypeptide and the fourth polypeptide, and/or a fourth polypeptide complex comprising the second polypeptide and the third polypeptide; and
recovering the third polypeptide complex and/or the fourth polypeptide complex;
wherein the destabilising modification of the CH3 domain of the first polypeptide or the second polypeptide does not destabilise association between the first polypeptide and the fourth polypeptide, and does not destabilise association between the second polypeptide and the third polypeptide;
and wherein the destabilising modification of the CH3 domain of the third polypeptide or the fourth polypeptide does not destabilise association between the first polypeptide and the fourth polypeptide, and does not destabilise association between the second polypeptide and the third polypeptide.

2. The method according to claim 1, wherein the destabilising modification of the CH3 domain of the first polypeptide or the second polypeptide stabilises association between the CH3 domain of the first polypeptide and the CH3 domain of the fourth polypeptide, and/or stabilises association between the CH3 domain of the second polypeptide and the CH3 domain of the third polypeptide.

3. The method according to claim 1 or claim 2, wherein the destabilising modification of the CH3 domain of the third polypeptide or the fourth polypeptide stabilises association between the CH3 domain of the first polypeptide and the CH3 domain of the fourth polypeptide, and/or stabilises association between the CH3 domain of the second polypeptide and the CH3 domain of the third polypeptide.

4. The method according to any one of claims 1 to 3, wherein the first polypeptide comprises a payload moiety and the fourth polypeptide comprises a functional moiety, or wherein the second polypeptide comprises a payload moiety and the third polypeptide comprises a functional moiety.

5. The method according to any one of claims 1 to 4, wherein:
(a) the CH3 domain of the first polypeptide comprises 370E, and the CH3 domain of the fourth polypeptide comprises 357K; optionally wherein the CH3 domain of the second polypeptide comprises 357E, and the CH3 domain of the third polypeptide comprises 370K; or
(b) the CH3 domain of the first polypeptide comprises 370K, and the CH3 domain of the fourth polypeptide comprises 357E; optionally wherein the CH3 domain of the second polypeptide comprises 357K, and the CH3 domain of the third polypeptide comprises 370E.

6. The method according to any one of claims 1 to 5, wherein:
(a) the CH3 domain of the first polypeptide comprises 366W and 370E, the CH3 domain of the second polypeptide comprises 407V, 366S, and 368A, the CH3 domain of the third polypeptide comprises 366W, and the CH3 domain of the fourth polypeptide comprises 407V, 366S, 368A and 357K; or
(b) the CH3 domain of the first polypeptide comprises 366W, 370E and 354C, the CH3 domain of the second polypeptide comprises 407V, 366S, and 368A, the CH3 domain of the third polypeptide comprises 366W, and the CH3 domain of the fourth polypeptide comprises 407V, 366S, 368A, 357K and 349C; or
(c) the CH3 domain of the first polypeptide has at least 70% amino acid sequence identity to SEQ ID NO:24, the CH3 domain of the second polypeptide has at least 70% amino acid sequence identity to SEQ ID NO:49, the CH3 domain of the third polypeptide has at least 70% amino acid sequence identity to SEQ ID NO:48, and the CH3 domain of the fourth polypeptide has at least 70% amino acid sequence identity to SEQ ID NO:25; or
(d) the CH3 domain of the first polypeptide has at least 70% amino acid sequence identity to SEQ ID NO:26, the CH3 domain of the second polypeptide has at least 70% amino acid sequence identity to SEQ ID NO:49, the CH3 domain of the third polypeptide has at least 70% amino acid sequence identity to SEQ ID NO:48, and the CH3 domain of the fourth polypeptide has at least 70% amino acid sequence identity to SEQ ID NO:27.

7. The method according to any one of claims 1 to 6, wherein the payload moiety is or comprises: a detectable moiety, a fluorescent moiety, a luminescent moiety, a radiopaque/contrast agent, a radiolabel, an immuno-detectable moiety, a moiety having a detectable activity, an enzymatic moiety, a drug moiety, or a cytotoxic moiety.

8. The method according to any one of claims 1 to 7, wherein the functional moiety is or comprises: a binding moiety, an antibody or a target-binding fragment or derivative thereof, a target-binding peptide/polypeptide, a target-binding nucleic acid, a detectable moiety, a fluorescent moiety, a luminescent moiety, a radiopaque/contrast agent, a radiolabel, an immuno-detectable moiety, a moiety having a detectable activity, an enzymatic moiety, a drug moiety or a cytotoxic moiety.

9. The method according to any one of claims 1 to 8, wherein the first polypeptide, the second polypeptide, the third polypeptide and/or the fourth polypeptide further comprise a CH2 domain and/or a hinge region.

10. A polypeptide complex according to the third polypeptide complex or the fourth polypeptide complex, produced by the method according to any one of claims 1 to 9.

11. A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide comprises a knob modification, and the CH3 domain of the second polypeptide comprises a hole modification;
wherein the CH3 domain of the first polypeptide comprises a destabilising modification, and wherein the CH3 domain of the second polypeptide comprises a destabilising modification;
wherein the destabilising modification of the CH3 domain of the first polypeptide does not destabilise association between the CH3 domain of the first polypeptide and the CH3 domain of the second polypeptide, and wherein the destabilising modification of the CH3 domain of the second polypeptide does not destabilise association between the CH3 domain of the first polypeptide and the CH3 domain of the second polypeptide; and
wherein the first polypeptide or the second polypeptide further comprises a payload moiety.

12. The polypeptide complex according to claim 11, wherein the first polypeptide and/or the second polypeptide further comprise a functional moiety.

13. The polypeptide complex according to claim 11 or claim 12, wherein the destabilising modification of the CH3 domain of the first polypeptide stabilises association between the CH3 domain of the first polypeptide and the CH3 domain of the second polypeptide; and/or wherein the destabilising modification of the CH3 domain of the second polypeptide stabilises association between the CH3 domain of the first polypeptide and the CH3 domain of the second polypeptide.

14. The polypeptide complex according to any one of claims 11 to 13, wherein the first polypeptide comprises a payload moiety and the second polypeptide comprises a functional moiety, or wherein the first polypeptide comprises a functional moiety and the second polypeptide comprises a payload moiety.

15. The polypeptide complex according to any one of claims 11 to 14, wherein:
(a) the CH3 domain of the first polypeptide comprises 366W and 370E; and the CH3 domain of the second polypeptide comprises 407V, 366S, 368A and 357K; or
(b) the CH3 domain of the first polypeptide comprises 366W, 370E and 354C; and the CH3 domain of the second polypeptide comprises 407V, 366S, 368A, 357K and 349C; or
(c) the CH3 domain of the first polypeptide has at least 70% amino acid sequence identity to SEQ ID NO:24; and the CH3 domain of the second polypeptide has at least 70% amino acid sequence identity to SEQ ID NO:25; or
(d) the CH3 domain of the first polypeptide has at least 70% amino acid sequence identity to SEQ ID NO:26; and the CH3 domain of the second polypeptide has at least 70% amino acid sequence identity to SEQ ID NO:27.

16. The polypeptide complex according to any one of claims 11 to 15, wherein the payload moiety is or comprises: a detectable moiety, a fluorescent moiety, a luminescent moiety, a radiopaque/contrast agent, a radiolabel, an immuno-detectable moiety, a moiety having a detectable activity, an enzymatic moiety, a drug moiety, or a cytotoxic moiety.

17. The polypeptide complex according to any one of claims 11 to 16, wherein the functional moiety is or comprises: a binding moiety, an antibody or a target-binding fragment or derivative thereof, a target-binding peptide/polypeptide, a target-binding nucleic acid, a detectable moiety, a fluorescent moiety, a luminescent moiety, a radiopaque/contrast agent, a radiolabel, an immuno-detectable moiety, a moiety having a detectable activity, an enzymatic moiety, a drug moiety or a cytotoxic moiety.

18. The polypeptide complex according to any one of claims 1 to 17, wherein the first polypeptide and/or the second polypeptide further comprise a CH2 domain and/or a hinge region.

19. A polypeptide complex, comprising a first polypeptide comprising a CH3 domain and a second polypeptide comprising a CH3 domain;
wherein the polypeptide complex is formed by interaction between the first polypeptide and the second polypeptide, the interaction comprising association between the CH3 domain of the first polypeptide and the CH3 domain of the second polypeptide;
wherein the CH3 domain of the first polypeptide comprises a knob modification, and the CH3 domain of the second polypeptide comprises a hole modification;
wherein the CH3 domain of the first polypeptide and/or the CH3 domain of the second polypeptide comprises a destabilising modification for destabilising association between the CH3 domain of the first polypeptide and the CH3 domain of the second polypeptide;
and wherein the first polypeptide and/or the second polypeptide further comprise a payload moiety.

20. The polypeptide complex according to claim 19, wherein:
(a) the CH3 domain of the first polypeptide comprises 370E, and the CH3 domain of the second polypeptide comprises 357E; or
(b) the CH3 domain of the first polypeptide comprises 370K, and the CH3 domain of the second polypeptide comprises 357K.

21. The polypeptide complex according to any one of claim 19 or claim 20, wherein:
(a) the CH3 domain of the first polypeptide comprises 366W and 370E; and the CH3 domain of the second polypeptide comprises 407V, 366S, and 368A; or
(b) the CH3 domain of the first polypeptide comprises 366W; and the CH3 domain of the second polypeptide comprises 407V, 366S, 368A and 357K; or
(c) the CH3 domain of the first polypeptide comprises 366W, 370E and 354C; and the CH3 domain of the second polypeptide comprises 407V, 366S, and 368A; or
(d) the CH3 domain of the first polypeptide comprises 366W; and the CH3 domain of the second polypeptide comprises 407V, 366S, 368A, 357K and 349C; or
(e) the CH3 domain of the first polypeptide comprises 366W and 370E; and the CH3 domain of the second polypeptide comprises 407V, 366S, 368A and 349C; or
(f) the CH3 domain of the first polypeptide comprises 366W and 354C; and the CH3 domain of the second polypeptide comprises 407V, 366S, 368A, and 357K; or
(g) the CH3 domain of the first polypeptide has at least 70% amino acid sequence identity to SEQ ID NO:48; and the CH3 domain of the second polypeptide has at least 70% amino acid sequence identity to SEQ ID NO:25; or
(h) the CH3 domain of the first polypeptide has at least 70% amino acid sequence identity to SEQ ID NO:26; and the CH3 domain of the second polypeptide has at least 70% amino acid sequence identity to SEQ ID NO:49; or
(i) the CH3 domain of the first polypeptide has at least 70% amino acid sequence identity to SEQ ID NO:48; and the CH3 domain of the second polypeptide has at least 70% amino acid sequence identity to SEQ ID NO:27; or
(j) the CH3 domain of the first polypeptide has at least 70% amino acid sequence identity to SEQ ID NO:24; and the CH3 domain of the second polypeptide has at least 70% amino acid sequence identity to SEQ ID NO:51; or
(k) the CH3 domain of the first polypeptide has at least 70% amino acid sequence identity to SEQ ID NO:50; and the CH3 domain of the second polypeptide has at least 70% amino acid sequence identity to SEQ ID NO:25.

22. The polypeptide complex according to any one of claims 19 to 21, wherein the payload moiety is or comprises: a detectable moiety, a fluorescent moiety, a luminescent moiety, a radiopaque/contrast agent, a radiolabel, an immuno-detectable moiety, a moiety having a detectable activity, an enzymatic moiety, a drug moiety, or a cytotoxic moiety.

23. The polypeptide complex according to any one of claims 19 to 22, wherein the first polypeptide and/or the second polypeptide further comprise a CH2 domain and/or a hinge region.
